(19) United States
(12) Patent Application Publication
(10) Pub. No.: US 2024/0382542 A1
(43) Pub. Date: Nov. 21, 2024

(54) FORMULATIONS TO STABILIZE VIRUS-BASED THERAPEUTICS

(71) Applicant: Boehringer Ingelheim International GmbH, Ingelheim am Rhein (DE)

(72) Inventors: Ludger Markus ICKENSTEIN, Biberach an der Riss (DE); Thomas KRIEHUBER, Maselheim (DE); Martin DASS, Biberach-Bergerhausen (DE); Julia SACHERL, Ochsenhausen (DE)

(21) Appl. No.: 18/665,671

(22) Filed: May 16, 2024

(30) Foreign Application Priority Data
May 17, 2023 (EP) 23173810.5

(51) Int. Cl. A61K 35/766; A61K 47/10; A61K 47/12; A61K 47/18; A61K 47/22; A61K 47/26; A61K 47/42

(52) U.S. Cl. CPC A61K 35/766; A61K 47/10; A61K 47/12; A61K 47/183; A61K 47/22; A61K 47/26; A61K 47/42

(57) ABSTRACT

The present invention relates to viral formulations for administration to a subject.

### SEQUENCE DISCLOSURE

This application includes, as part of its disclosure, a “Sequence Listing XML” pursuant to 37 C.F.R. § 1.831 (a) which is submitted in XML file format via the USPTO patent electronic filing system in a file named “01-3568-US-1_SL.xml” created on Jun. 11, 2024, and having a size of 46,832 bytes, which is hereby incorporated by reference herein in its entirety.

### FIELD OF THE INVENTION

The present invention relates to a pharmaceutical formulation for virus-based therapeutics and virus-based cancer vaccines. More specifically, the present invention relates to a pharmaceutical formulation for oncolytic viruses as disclosed herein.

### BACKGROUND OF THE INVENTION

Oncolytic viruses, such as the vesicular stomatitis virus (VSV) containing the glycoprotein (GP) of the lymphocytic chorio-meningitis virus (VSV-GP), are an emerging class of biologicals, which selectively replicate in and kill cancer cells. The oncolytic viruses described herein can spread within tumors and efficiently induce tumor cell lysis resulting in cell death. Furthermore, additional genes may be cloned into the virus genome and expression of said proteins can stimulate and/or direct the immune system against tumor cells. In addition, oncolytic viruses expressing cancer-specific antigens can be co-administered together with the antigen and thus enhance and prolong its immune-stimulating effect. Accordingly, oncolytic viruses are beneficial for the treatment and/or the prevention of cancer.

The development of a formulation that stabilizes oncolytic viruses upon storage is challenging. Isolated concentrated live viruses are typically unstable during long-term storage and sensitive towards elevated temperatures, mechanical stress, freeze-thaw cycles, and the like. To be effective as a therapeutic agent, live viruses need to be formulated to preserve the activity and to prevent aggregation and thus, the formation of visible and subvisible particles (SvPs). Owing to their structural complexity, viruses and in particular VSV-GP tends to self-associate and aggregate. The colloidal stability of virus particles must be ensured, and aggregation must be reduced below compendial limits by formulation development efforts. The number of freezing and thawing events, for instance for filling and labelling of vials, as well as the freezing and storage conditions, such as the freezing and thawing rate and the storage temperature impact greatly the infectivity and colloidal stability especially when viruses are not stored as a frozen liquid at temperatures below the glass transition temperature of the maximally freeze-concentrated solution (T_{g}′).

Although several formulations for virus-based therapeutics are described in the literature for specific viruses, it is commonly understood that each virus species, genus, or family needs to be formulated in its own specific formulation.

Accordingly, there is a need for stable liquid, frozen liquid, and dry formulations (such as lyophilized formulations) for virus-based therapeutics and in particular formulations for oncolytic viruses, such as VSV, in particular VSV-GP, which are suitable for parenteral administration, including intravenous or intratumoral injection into human patients. There exists furthermore a need for formulations that exhibit e.g., increased colloidal stability and no or only minor loss of the biological activity of the therapeutic virus during extended periods of storage at various temperatures. There is a particular need for formulations for lyophilized virus-based therapeutics that open the possibility that drug product vials can be stored at room temperature or for longer time periods at a temperature of 5° C. with reduced sensitivities towards temperature fluctuations that can occur within the cold-chain from the manufacturing site to the bedside.

### SUMMARY OF THE INVENTION

The present invention addresses the needs mentioned above by providing stable formulations and in particular stable dry formulations (such as lyophilized formulations) for virus-based therapeutics.

In a first aspect the invention relates to a pharmaceutical composition comprising an enveloped virus, one or more sugar alcohol, AND a protein agent and/or a poly(ethylene oxide)/poly(propylene oxide) block copolymer.

In one embodiment relating to the first aspect, the poly(ethylene oxide) and poly(propylene oxide) block copolymer is a poloxamer. In one embodiment relating to the first aspect, the poly(ethylene oxide) and poly(propylene oxide) block copolymer is poloxamer 188.

In one embodiment relating to the first aspect, the protein agent is albumin or gelatin. In one embodiment relating to the first aspect, the protein agent is human serum albumin or recombinant human albumin.

In one embodiment relating to the first aspect, the poly(ethylene oxide) and poly(propylene oxide) block copolymer is poloxamer 188, and the protein agent is human serum albumin or recombinant human albumin. In a related embodiment, the pharmaceutical composition comprises poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L. In a further related embodiment, the pharmaceutical composition comprises human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

In one embodiment relating to the first aspect and any of its embodiments, the pharmaceutical composition further comprises at least one of an amino acid, a buffer, or a sugar. In a related embodiment, the amino acid is selected from the group consisting of alanine, arginine, phenylalanine, glutamic acid, glycine, methionine, lysine, and glutamine, preferably glutamic acid and/or arginine. In a related embodiment, the buffer is selected from the group consisting of acetate, citrate, histidine, succinate, HEPES, tartrate, phosphate, citrate/phosphate, lactate and Tris, preferably Tris. In a related embodiment, the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM. In a related embodiment, the sugar is selected from the group consisting of dextrose, fructose, galactose, glucose, raffinose, trehalose, and sucrose, preferably trehalose. In a related embodiment, the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

In one embodiment relating to the first aspect and any of its embodiments, the one or more sugar alcohol is selected from the group consisting of: mannitol, sorbitol, xylitol, maltitol, maltitol symp, lactitol, inositol, glycerol erythritol, isomalt, and hydrogenated starch hydroxylate. In a further related embodiment, the one or more sugar alcohol is mannitol and/or sorbitol, preferably a combination of mannitol and sorbitol.

In one embodiment relating to the first aspect, the composition is substantially free of chloride, preferably substantially free of NaCl.

In one embodiment relating to the first aspect, the pH of the composition is between 5 to 9, or between 6 to 9, or between 6.5 to 8.5, or between 6.5 to 8.0, preferably between 7.0 and 8.0. In a related embodiment, the pH of the composition is adjusted with phosphoric acid, lactic acid, citric acid, succinate acid or sodium phosphate.

In one embodiment relating to the first aspect, the pharmaceutical composition comprises:
an enveloped virus,
one or more sugar alcohol,
at least one of a buffer, an amino acid or a sugar,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a sugar,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer and a sugar,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
a buffer and a sugar,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
at least one of a buffer, an amino acid or a sugar,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer, wherein the buffer is selected from the group consisting of acetate, citrate, histidine, succinate, HEPES, tartrate, phosphate, citrate/phosphate, lactate and Tris, preferably Tris,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer and a sugar, wherein the buffer is selected from the group consisting of acetate, citrate, histidine, succinate, HEPES, tartrate, phosphate, citrate/phosphate, lactate and Tris, preferably Tris,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
a buffer and a sugar, wherein the buffer is selected from the group consisting of acetate, citrate, histidine, succinate, HEPES, tartrate, phosphate, citrate/phosphate, lactate and Tris, preferably Tris,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
at least one of a buffer, an amino acid or a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer, wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer and a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
a buffer and a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
at least one of a buffer, an amino acid or a sugar, wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a sugar, wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer and a sugar, wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a sugar, wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
an amino acid and a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol,
a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
at least one of a buffer, an amino acid or a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a buffer, wherein the amino acid is selected from the group consisting of alanine, arginine, phenylalanine, glutamic acid, glycine, methionine, lysine, and glutamine, preferably glutamic acid and/or arginine,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a buffer and a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
a buffer and a sugar, wherein the sugar is selected from the group consisting of dextrose, fructose, galactose, glucose, raffinose, trehalose, and sucrose, preferably trehalose,

### AND

a protein agent and/or a poly(ethylene oxide)/poly(propylene oxide) block copolymer,
   wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
at least one of a buffer, an amino acid or a sugar, wherein the amino acid is selected from the group consisting of alanine, arginine, phenylalanine, glutamic acid, glycine, methionine, lysine, and glutamine, preferably glutamic acid and/or arginine,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a sugar, wherein the amino acid is selected from the group consisting of alanine, arginine, phenylalanine, glutamic acid, glycine, methionine, lysine, and glutamine, preferably glutamic acid and/or arginine,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a buffer and a sugar, wherein the amino acid is selected from the group consisting of alanine, arginine, phenylalanine, glutamic acid, glycine, methionine, lysine, and glutamine, preferably glutamic acid and/or arginine,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a buffer, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a sugar, wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
an amino acid and a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
a buffer, wherein the buffer is Tris

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
an amino acid, wherein the amino acid is arginine or glutamic acid,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
a sugar, wherein the sugar is trehalose or sucrose,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
an amino acid, wherein the amino acid is arginine or glutamic acid,
a sugar, wherein the sugar is trehalose or sucrose,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
a buffer, wherein the buffer is Tris
a sugar, wherein the sugar is trehalose or sucrose,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
a buffer, wherein the buffer is Tris
an amino acid, wherein the amino acid is arginine or glutamic acid,

### AND

wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
mannitol and/or sorbitol
a buffer, wherein the buffer is Tris
an amino acid, wherein the amino acid is arginine or glutamic acid,
a sugar, wherein the sugar is trehalose or sucrose,

### AND

wherein the protein agent is selected from human serum albumin or recombinant human albumin and the poly(ethylene oxide)/poly(propylene oxide) block copolymer is poloxamer 188.

In one embodiment relating to the first aspect and any of its embodiments comprising a buffer, the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM.

In one embodiment relating to the first aspect and any of its embodiments comprising a sugar, the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

In one embodiment relating to the first aspect and any of its embodiments, the pharmaceutical composition comprises poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

In one embodiment relating to the first aspect and any of its embodiments, the pharmaceutical composition comprises human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

In one embodiment relating to the first aspect, the pharmaceutical composition comprises:
an enveloped virus,
about 1-100 mM Tris,
about 1-100 mM glutamic acid,
about 10-500 mM trehalose,
about 10-200 mM mannitol,
about 10-200 mM sorbitol,
about 0.1-5 mg/ml poloxamer 188,
about 0.5-10 mg/ml recombinant human albumin, and
a pH of about 6 to 8.

In one embodiment relating to the first aspect, the pharmaceutical composition comprises:
an enveloped virus,
about 1-100 mM Tris,
about 1-100 mM glutamic acid,
about 10-500 mM arginine,
about 10-500 mM trehalose,
about 10-200 mM mannitol,
about 10-200 mM sorbitol,
about 0.1-5 mg/ml poloxamer 188,
about 0.5-10 mg/ml recombinant human albumin, and
a pH of about 6 to 8.

In one embodiment relating to the first aspect, the pharmaceutical composition comprises:
an enveloped virus,
about 1-100 mM Tris,
about 1-100 mM glutamic acid,
about 10-500 mM trehalose,
about 1-100 mM citrate,
about 10-200 mM mannitol,
about 10-200 mM sorbitol,
about 0.1-5 mg/ml poloxamer 188,
about 0.5-10 mg/ml recombinant human albumin, and
a pH of about 6 to 8.

In one embodiment relating to the first aspect, the pharmaceutical composition comprises:
an enveloped virus,
about 1-100 mM Tris,
about 1-100 mM glutamic acid,
about 10-500 mM trehalose,
about 10-200 mM mannitol,
about 10-200 mM sorbitol,
about 0.1-5 mg/ml poloxamer 188,
about 0.5-10 mg/ml recombinant human albumin, and
a pH of about 6 to 8.

In one embodiment relating to the first aspect, the pharmaceutical composition comprises:
an enveloped virus
about 10 mM Tris,
about 200 mM trehalose,
about 20 mM glutamic acid,
about 50 mM mannitol,
about 50 mM sorbitol,
about 1.275 mg/ml poloxamer 188,
about 5 mg/ml recombinant human albumin, and
a pH of about 7.4.

In one embodiment relating to the first aspect, the pharmaceutical composition comprises:
an enveloped virus
about 10 mM Tris,
about 125 mM trehalose,
about 20 mM glutamic acid,
about 100 mM mannitol,
about 40 mM sorbitol,
about 100 mM arginine,
about 2.5 mg/ml poloxamer 188,
about 5 mg/ml recombinant human albumin, and
a pH of about 7.1.

In one embodiment relating to the first aspect, the pharmaceutical composition comprises:
an enveloped virus,
about 10 mM Tris,
about 20 mM glutamic acid,
about 200 mM trehalose,
about 25 mM citrate,
about 50 mM mannitol,
about 50 mM sorbitol,
about 2.5 mg/ml poloxamer 188,
about 5 mg/ml recombinant human albumin, and
a pH of about 7.4.

In one embodiment relating to the first aspect, the pharmaceutical composition comprises:
an enveloped virus
about 10 mM Tris,
about 200 mM trehalose,
about 20 mM glutamic acid,
about 50 mM mannitol,
about 50 mM sorbitol,
about 150 mM arginine,
about 1.275 mg/ml poloxamer 188,
about 5 mg/ml recombinant human albumin, and
a pH of about 7.4.

In one embodiment relating to the first aspect, the pharmaceutical composition comprises:
an enveloped virus
about 10 mM Tris,
about 133 mM trehalose,
about 13 mM glutamic acid,
about 33 mM mannitol,
about 33 mM sorbitol,
about 100 mM arginine,
about 1.275 mg/ml poloxamer 188,
about 5 mg/ml recombinant human albumin, and
a pH of about 7.4.

In one embodiment relating to the first aspect and any of its embodiments, the pH of the composition is adjusted with phosphoric acid or sodium phosphate or lactic acid.

In one embodiment relating to the first aspect and any of its embodiments, the enveloped virus is a rhabdoviridae, preferably a vesiculovirus or vesicular stomatitis virus (VSV). In a related embodiment, the enveloped virus is a recombinant vesicular stomatitis virus (VSV), wherein the gene coding for the glycoprotein G of the vesicular stomatitis virus is replaced by the gene coding for the glycoprotein GP of LCMV, and/or the glycoprotein G is replaced by the glycoprotein GP of LCMV. In a related embodiment, the pharmaceutical composition comprises the enveloped virus, preferably the vesiculovirus or vesicular stomatitis virus (VSV), in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, at least 1×10⁹ TCID₅₀/mL, or at least 1×10⁹ TCID₅₀/mL. In a further related embodiment, the pharmaceutical composition comprises the enveloped virus, preferably the vesiculovirus or vesicular stomatitis virus (VSV), in a concentration range between 1×10⁵ TCID₅₀/mL to 1×10¹² TCID₅₀/mL, between 1×10⁶ TCID₅₀/mL to 1×10¹² TCID₅₀/mL, between 1×10⁷ TCID₅₀/mL to 1×10¹² TCID₅₀/mL, between 1×10⁸ TCID₅₀/mL to 1×10¹² TCID₅₀/mL, 1×10⁵ TCID₅₀/mL to 1×10¹¹ TCID₅₀/mL, 1×10⁵ TCID₅₀/mL to 1×10¹⁰ TCID₅₀/mL, or 1×10⁵ TCID₅₀/mL to 1×10⁹ TCID₅₀/mL.

In one embodiment relating to the first aspect and any of its embodiments, the pharmaceutical composition is a liquid or frozen liquid pharmaceutical composition.

In one embodiment relating to the first aspect and any of its embodiments, a dry pharmaceutical composition is produced by a method comprising removing water from the pharmaceutical composition.

In one embodiment relating to the first aspect and any of its embodiments, the pharmaceutical composition is a dry pharmaceutical composition. In a related embodiment, the dry pharmaceutical composition is obtained by freeze-drying/lyophilization, or spray-drying. In a related embodiment, the method comprises placing the pharmaceutical composition in a vacuum under controlled temperatures and pressure to remove the water. In a related embodiment, the method is lyophilization. In a related embodiment, the dry pharmaceutical composition comprises less than about (0.1%-10%) w/w water. In a related embodiment, a pharmaceutical composition comprises water and the dry pharmaceutical composition. A further embodiment relates to a pharmaceutical composition comprising water and the product.

In a further embodiment, the lyophilized formulation that, when reconstituted in water, results in a reconstituted solution comprising:
an enveloped virus,
mannitol in a concentration between 10 mM to 200 mM and/or sorbitol in a concentration between 10 mM to 200 mM,

### AND

poloxamer 188 in a concentration between 0.01 g/L to 50 g/L and/or recombinant human albumin in a concentration between 0.1 g/L to 50 g/L.

### BRIEF DESCRIPTION OF THE FIGURES

FIG. 1 Geometric mean values of VSV-GP-Cargo1 infectivity following storage of liquid formulations at a temperature of 5° C. Error bars indicate the standard deviation (n=2 for control, n=3 for others, measured in 3 replicas, each). A frozen crude harvest sample serves as a control.

FIG. 2 Geometric mean values of VSV-GP-Cargo1 infectivity following storage of liquid formulations at a temperature of 25° C. Error bars indicate the standard deviation (n=2 for control, n=3 for others, measured in 3 replicas, each). A frozen crude harvest sample serves as a control.

FIG. 3 Geometric mean values of SvPs≥10 μm of VSV-GP-Cargo1 following storage of liquid formulations at a temperature of 5° C. Error bars indicate the standard deviation (n=2 for control, n=3 for others, measured in 3 replicas, each).

FIG. 4 Geometric mean values of SvPs≥10 μm of VSV-GP-Cargo1 following storage of liquid formulations at a temperature of 25° C. Error bars indicate the standard deviation (n=2 for control, n=3 for others, measured in 3 replicas, each).

FIG. 5 Geometric mean values of VSV-GP-Cargo1 infectivity following no, one, or three freeze/thaw cycles and storage of liquid formulations at a temperature of 25° C. Error bars indicate the standard deviation (n=2 for control, n=3 for others, measured in 3 replicas, each). A frozen crude harvest sample serves as a control.

FIG. 6 Geometric mean values of SvPs≥10 μm of VSV-GP-Cargo1 following no, one, or three freeze/thaw cycles of liquid formulations. Error bars indicate the standard deviation (n=2 for control, n=3 for others, measured in 3 replicas, each).

FIG. 7 Geometric mean values of VSV-GP-Cargo1 infectivity following 1, 3, or 5 freeze/thaw cycles at temperatures of −20° C. and +25° C. of frozen liquid formulations. Error bars indicate the standard deviation (n=2 for control, n=3 for others, measured in 3 replicas, each). A frozen crude harvest sample serves as a control.

FIG. 8 Geometric mean values of VSV-GP-Cargo1 infectivity following 1, 3, or 5 freeze/thaw cycles at temperatures of −80° C. and +25° C. of frozen liquid formulations. Error bars indicate the standard deviation (n=2 for control, n=3 for others, measured in 3 replicas, each). A frozen crude harvest sample serves as a control.

FIG. 9 Geometric mean values of SvPs≥10 μm of VSV-GP-Cargo1 following 1, 3, or 5 freeze/thaw cycles at temperatures of −20° C. and +25° C. of frozen liquid formulations. Error bars indicate the standard deviation (n=2 for control, n=3 for others, measured in 3 replicas, each).

FIG. 10 Geometric mean values of SvPs≥10 μm of VSV-GP-Cargo1 following 1, 3, or 5 freeze/thaw cycles at temperatures of −80° C. and +25° C. of frozen liquid formulations. Error bars indicate the standard deviation (n=2 for control, n=3 for others, measured in 3 replicas, each).

FIG. 11 Geometric mean values of VSV-GP-Cargo1 infectivity following storage of lyophilized formulations at a temperature of 25° C. Error bars indicate the standard deviation (n=2 for control, n=3 for others, measured in 3 replicas, each). As a control serves a frozen crude harvest sample. The first time point represents the measurement before lyophilization. The second value (0 weeks) is the value immediately after lyophilisation and reconstitution.

FIG. 12 Geometric mean values of SvPs≥10 μm of VSV-GP-Cargo1 following storage of lyophilized formulations at a temperature of 25° C. Error bars indicate the standard deviation (n=2 for control, n=3 for others, measured in 3 replicas, each). The first time point represents the measurement before lyophilization. The second value is the value immediately after lyophilisation and reconstitution.

FIG. 13 T_{g} values of WP7 placebo formulations.

FIG. 14 Lyo cakes of WP7 formulations before and after storage for 14 days at a temperature of 5° C. or at 25° C.

FIGS. 15A-B A) Onset and midpoint temperatures of a second melting transition (T_{g}) peak in lyophilized WP7 placebo formulations. B) Onset and midpoint temperatures of a second glass transition (T_{g}′) peak in reconstituted WP7 placebo formulations. Formulations 1, 2, and 4 did not contain poloxamer 188.

FIG. 16 Geometric mean values of VSV-GP-Cargo1 infectivity following storage of lyophilized formulations at a temperature of 25° C. Error bars indicate the standard deviation (n=2 measured in 3 replicas, each). The first time point represents the measurement before lyophilization. The second value is the value immediately after lyophilisation and reconstitution. A frozen crude harvest sample serves as a control.

FIG. 17 Geometric mean values of VSV-GP-Cargo1 infectivity following storage of lyophilized formulations at a temperature of 5° C. Error bars indicate the standard deviation (n=2 measured in 3 replicas, each). The first time point represents the measurement before lyophilization. The second value is the value immediately after lyophilisation and reconstitution. A frozen crude harvest sample serves as a control.

FIG. 18 Geometric mean values of VSV-GP-Cargo1 infectivity following storage of lyophilized formulations at a temperature of 25° C. Error bars indicate the standard deviation (n=2 measured in 3 replicas, each). The first time point represents the measurement before lyophilization. The second value is the value immediately after lyophilisation and reconstitution. The 6 months and 12 months values of WP09a_03, WP09_04 were below the limit of detection. A frozen crude harvest sample serves as a control.

FIG. 19 Geometric mean values of VSV-GP-Cargo2 infectivity following storage of lyophilized formulations at a temperature of 5° C. Error bars indicate the standard deviation (n=2 measured in 3 replicas, each). The first time point represents the measurement before lyophilization. The second value is the value immediately after lyophilisation and reconstitution. A frozen crude harvest sample serves as a control.

FIG. 20 Geometric mean values of VSV-GP-Cargo2 infectivity following storage of lyophilized formulations at a temperature of 25° C. The first time point represents the measurement before lyophilization. The second value is the value immediately after lyophilisation and reconstitution. The 6 months and 12 months values of WP09_05 were below the limit of detection. A frozen crude harvest sample serves as a control.

FIGS. 21A-B Geometric mean values of VSV-GP-Cargo1 infectivity following storage of lyophilized formulations at a temperature of 25° C. Error bars indicate the standard deviation (n=2 measured in 3 replicas, each). The first time point represents the measurement before lyophilization. The second value is the value immediately after lyophilisation and reconstitution. The 3 months, 6 months, and 12 months values of WP10_01, WP10_06, and WP10_09 were below the limit of detection. A frozen crude harvest sample serves as a control.

FIGS. 22A-D Geometric mean values of VSV-GP-Cargo1 infectivity following storage of lyophilized formulations at a temperature of 25° C. in a Design of Experiment (DoE) approach. Error bars indicate the standard deviation (n=4 replicas, each). The first value is the value immediately after lyophilisation and reconstitution. The 12 months values of WP10_06 and WP10_09 were below the limit of detection. A frozen crude harvest sample serves as a control.

FIGS. 23A-B (A) Geometric mean values of VSV-GP infectivity following storage of lyophilized formulations at a temperature of 2-8° C. Error bars indicate the standard deviation (3 to 9 replicas, each). The first value (t=0) is the value immediately after lyophilization and reconstitution. (B) Mean total particle concentration measured by NTA after liquid processing of five different formulations containing VSV-GP. Each point indicates one sample (5 acquisitions, each) Error bars indicate standard deviation (n=2 with 5 acquisitions, each).

### DETAILED DESCRIPTION OF THE INVENTION

In the following detailed description, numerous specific details are set forth to provide a full understanding of the present invention. It will be apparent, however, to a person ordinarily skilled in the art that the subject technology may be practiced without some of these specific details. In other instances, well-known structures and techniques have not been shown in detail so as not to obscure the present invention. The headings are included merely for convenience to assist in reading and shall not be understood to limit the invention to specific aspects or embodiments.

In one aspect, a formulation of the present invention is useful to stabilize the infectious titer of an enveloped virus. In another aspect, the formulation helps to preserve the infectious titer and/or the colloidal stability of an enveloped virus after one or more freeze-thaw cycles. In a related aspect, the formulation is useful to preserve the infectious titer and/or colloidal stability of an enveloped virus upon storage at elevated temperatures, preferably at a temperature of 2-8° C., at room temperature, or even at temperatures above room temperature. In a related aspect, the formulation is useful to preserve titer and/or activity of an enveloped virus for storage of the virus at various temperatures for certain periods of time.

In another aspect, a formulation of the present invention may reduce, and/or slow down the formation of visible and/or subvisible particles (SvPs) in formulations containing the enveloped virus. In a related aspect, the formulation containing the enveloped virus has a reduced amount of visible and/or SvPs.

In yet another aspect, a formulation of the present invention helps to preserve the infectious titer of an enveloped virus. In a related aspect, the infectious titer is retained or the loss in infectious titer over time is slowed down. In a related aspect, the loss in infectious titer due to prolonged storage, repeated freeze-thaw cycles, storage at elevated temperatures or mechanical stress is slowed down.

In another aspect, a formulation of the present invention helps to suppress, slow down, or prevent aggregation of an enveloped virus. In another aspect, the formulation is useful to suppress, reduce, or prevent clouding of a formulation containing the enveloped virus.

In another aspect, a formulation of the present invention helps to suppress, slow down, or prevent the formation of visible and/or SvPs within a formulation containing an enveloped virus. Preferably, the formulation containing the enveloped virus is substantially free of visible and/or SvPs.

In another aspect, a formulation of the present invention containing an enveloped virus preserves the infectivity of the enveloped virus and/or slows down the loss of infectivity upon storage at a temperature of 25° C.

In another aspect, the formulation is useful to preserve the infectious titer and/or the colloidal stability of an enveloped virus upon storage at a temperature of 2-8° C.

In another aspect, a formulation of the present invention containing an enveloped virus has visible/sub-visible particle concentrations that are below the compendial limits after reconstitution.

In an aspect relating to any of the aforementioned aspects, the formulation containing the enveloped virus is a liquid formulation, a frozen liquid formulation, or a dry formulation such as a lyophilized formulation.

In another aspect, a dry formulation of the present invention containing an enveloped virus is further stabilized by increasing the T_{g} values of the formulation by removing NaCl and adjusting the pH of the formulation with phosphoric acid or sodium phosphate, such as trisodium phosphate (Na₃PO₄) and/or phosphoric acid (H₃PO₄) or lactate and/or lactic acid instead of sodium hydroxide (NaOH) and/or hydrochloric acid (HCl).

In another aspect, a dry formulation of the present invention containing an enveloped virus preserves the infectivity of the enveloped virus and/or slows down loss of infectivity upon storage at a temperature of 25° C. for at least 1, 2, 3, 6, or at least 12 months.

In another aspect, a dry formulation of the present invention containing an enveloped virus can be stored for at least 1, 2, 3, 6, or at least 12 months at a temperature of 25° C. or 5° C. In a related aspect, overall TCID₅₀ loss is less than 0.25-0.3 log units. In a further related aspect, the visible/sub-visible particle concentrations are below the compendial limits after reconstitution.

In another aspect, adjustment of formulation buffer with trisodium phosphate (Na₃PO₄) and/or phosphoric acid (H₃PO₄) together with omission of sodium chloride reduces the osmolality of the reconstituted formulation and increases the T_{g} value of the maximally freeze-concentrated solution.

In another aspect, adjustment of formulation buffer with phosphoric acid (H₃PO₄) instead of HCl reduces osmolality in formulations without trehalose and increases T_{g} value by approximately 10° C. to 20° C.

In another aspect, omission of sodium chloride or in general chloride ions in the formulation eliminates formation of chloride salts upon freezing.

In relation to any of the aforementioned aspects, the titer of a particular formulation can be tested by determining the TCID₅₀ according to the method as shown in the Examples. In relation to any of the aforementioned aspects, the particles can be assessed according to the method as described in the Examples.

Without being bound by theory, it has been found that poloxamer and particular poloxamer 188 reduces the formation of aggregates and/or particles in liquid and/or frozen liquid oncolytic virus formulations, in particular following freeze/thaw cycling. Even more surprisingly, poloxamer 188 was able to stabilize infectivity after storage of lyophilized oncolytic virus formulations at various temperatures. This was particularly surprising as poloxamer is known to act as a detergent. However, in contrast to other detergents, such as Tween, poloxamer did not destroy the virus or reduced its infectivity. Moreover, the presence of poloxamer and in particular poloxamer 188 in the formulation was shown to stabilize the infectivity of lyophilized virus upon storage at various temperatures.

It was also found that recombinant human albumin (rHA) was able to reduce the formation of aggregates and/or particles in frozen liquid and lyophilized formulations, presumably by interaction with the virus surface. Moreover, rHA was found to stabilize the infectivity of lyophilized virus upon storage in frozen liquid and lyophilized formulations.

Surprisingly, the combination of both, recombinant human albumin and poloxamer 188, showed an additive/synergistical effect on the infectivity of lyophilized virus upon storage. This effect was accompanied by further reduction of the formation of particles. It was also found that in liquid and/or frozen liquid formulations stress induced particles in rHA containing formulations could be effectively prevented by the addition of poloxamer 188.

The pharmaceutical formulations and compositions described herein are particularly useful for the formulation of enveloped viruses. According to the present invention, the term “enveloped virus” refers to any of the genera of enveloped viruses capable of infecting humans, such as but not limited to herpesvirus, poxvirus, orthomyxovirus, paramyxoviruses, rhabdovirus, and filoviruses. As used herein a rhabdovirus can belong to the genus of: almendravirus, curiovirus, cytorhabdovirus, dichorhavirus, ephemerovirus, hapavirus, ledantevirus, lyssavirus, novirhabdovirus, nucleorhabdovirus, perhabdovirus, sigmavirus, sprivivirus, sripuvirus, tibrovirus, tupavirus, varicosavirus, or vesiculovirus.

Preferably, the enveloped virus is replication competent. Further preferred are oncolytic viruses and replication competent oncolytic viruses. In this respect, the term oncolytic is meant in its regular meaning, which is known in the art and refers to the ability of a virus to infect and lyse (break down) cancer cells but not normal cells (to any significant extend). Preferably, the oncolytic virus is capable of replication within cancer cells. Oncolytic activity may be tested in different assay systems known to the skilled artisan (an exemplary in vitro assay is described by Muik et al., Cancer Res., 74 (13), 3567-78, 2014). It is understood that an oncolytic virus may infect and lyse only specific types of cancer cells. Also, the oncolytic effect may vary depending on the type of cancer cells. It is understood that an oncolytic virus is a live virus that is able to infect and replicate in cancer cells.

The enveloped virus of any of the embodiments can be a recombinant or non-recombinant enveloped virus, preferably a recombinant enveloped virus, preferably a recombinant enveloped virus belonging to the family of rhabdoviridae, more preferably a recombinant vesiculovirus, and even more preferably a recombinant vesicular stomatitis virus.

The term “recombinant” refers to a virus, more particularly an enveloped virus, comprising an exogenous nucleic acid sequence inserted in its genome, which is not naturally present in the parent virus. A recombinant virus thus refers to a nucleic acid or virus made by an artificial combination of two or more segments of nucleic acid sequence of synthetic or semisynthetic origin which does not occur in nature or is linked to another nucleic acid in an arrangement not found in nature. The artificial combination is most commonly accomplished by artificial manipulation of isolated segments of nucleic acids, using well-established genetic engineering techniques. Generally, a “recombinant” enveloped virus as described herein refers to enveloped viruses that are produced by standard genetic engineering methods, e.g., enveloped viruses of the present invention are thus genetically engineered or genetically modified enveloped virus. The term “recombinant enveloped virus” thus includes enveloped viruses, which have stably integrated recombinant nucleic acid in their genome.

Characterizing features of members of the family of rhabdoviruses include a negative-sense, single-stranded RNA of 10.8-16.1 kb, which are mostly unsegmented and a genome encoding for at least 5 genes encoding the structural proteins nucleoprotein (N), large protein (L), phosphoprotein (P), matrix protein (M), and glycoprotein (G).

Vesiculovirus species have been defined primarily by serological means coupled with phylogenetic analysis of the genomes. Biological characteristics such as host range and mechanisms of transmission are also used to distinguish viral species within the genus. As such, the genus of vesiculovirus form a distinct monophyletic group well-supported by maximum likelihood trees inferred from complete L sequences.

Viruses assigned to different species within the genus vesiculovirus may have one or more of the following characteristics: A) a minimum amino acid sequence divergence of 20% in L; B) a minimum amino acid sequence divergence of 10% in N; C) a minimum amino acid sequence divergence of 15% in G; D) can be distinguished in serological tests; and E) occupy different ecological niches as evidenced by differences in hosts and or arthropod vectors.

In a preferred embodiment the vesicular stomatitis virus encodes in its genome at least for a vesicular stomatitis virus nucleoprotein (N), large protein (L), phosphoprotein (P), matrix protein (M), glycoprotein (G).

In a preferred embodiment the vesicular stomatitis virus encodes in its genome at least for a vesicular stomatitis virus nucleoprotein (N) comprising an amino acid sequence as set forth in SEQ ID NO:1 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:1, a phosphoprotein (P) comprising an amino acid sequence as set forth in SEQ ID NO:2 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:2, a large protein (L) comprising an amino acid sequence as set forth in SEQ ID NO:3 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:3, and a matrix protein (M) comprising an amino acid sequence as set forth in SEQ ID NO:4 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4.

It is understood by the skilled artisan that modifications to the vesicular stomatitis virus nucleoprotein (N), large protein (L), phosphoprotein (P), matrix protein (M), or glycoprotein (G) sequence can be made without losing the basic functions of those proteins. Such functional variants as used herein retain all or part of their basic function or activity. The protein L for example is the polymerase and has an essential function during transcription and replication of the virus. A functional variant thereof must retain at least part of this ability. A good indication for retention of basic functionality or activity is the successful production of viruses, including these functional variants, that are still capable to replicate and infect tumor cells. Production of viruses and testing for infection and replication in tumor cells may be tested in different assay systems known to the skilled artisan (an exemplary in vitro assay is described by Muik et al., Cancer Res., 74 (13), 3567-78, 2014).

In a preferred embodiment the vesicular stomatitis virus encodes in its genome at least for a vesicular stomatitis virus nucleoprotein (N), large protein (L), phosphoprotein (P), matrix protein (M) and glycoprotein (G), wherein the large protein (L) comprises an amino acid sequence having a sequence identity ≥80% of SEQ ID NO: 3.

In a preferred embodiment the vesicular stomatitis virus encodes in its genome at least for a vesicular stomatitis virus nucleoprotein (N), large protein (L), phosphoprotein (P), matrix protein (M) and glycoprotein (G), wherein the nucleoprotein (N) comprises an amino acid sequence having a sequence identity >90% of SEQ ID NO:1.

In a further preferred embodiment, the vesicular stomatitis virus encodes in its genome at least for a vesicular stomatitis virus nucleoprotein (N), large protein (L), phosphoprotein (P), matrix protein (M) and glycoprotein (G), wherein the large protein (L) comprises an amino acid sequence having a sequence identity ≥80% of SEQ ID NO: 3 and the nucleoprotein (N) comprises an amino acid sequence having a sequence identity ≥90% of SEQ ID NO:1.

It is known that certain wildtype VSV strains are considered to be neurotoxic. It is also reported that infected individuals are able to rapidly mount a strong humoral response with high antibody titers directed mainly against the glycoprotein. Neutralizing antibodies targeting the glycoprotein G of VSV are able to limit virus spread and thereby mediate protection of individuals from virus re-infection. Virus neutralization, however, limits repeated application of the virus to the cancer patient.

To eliminate these drawbacks the wildtype glycoprotein G may be replaced with the glycoprotein from another virus. In this respect replacing the glycoprotein refers to (i) replacement of the gene coding for the wild-type glycoprotein G with the gene coding for the glycoprotein GP of another virus, and/or (ii) replacement of the wild-type glycoprotein G with the glycoprotein GP of another virus.

In a preferred embodiment the enveloped virus is a vesicular stomatitis virus and the VSV glycoprotein G is replaced with the glycoprotein GP of the lymphocytic choriomeningitis virus (LCMV), preferably with the glycoprotein of the strain WE-HPI. Such VSV is for example described in WO2010/040526 and named VSV-GP.

Hence, in a most preferred embodiment the enveloped virus is a recombinant vesicular stomatitis virus, wherein the gene coding for the glycoprotein G of the vesicular stomatitis virus is replaced by the gene coding for the glycoprotein GP of LCMV, and/or the glycoprotein G is replaced by the glycoprotein GP of LCMV.

In a preferred embodiment, the gene coding for the glycoprotein GP of the LCMV encodes for a protein with an amino acid sequence as shown in SEQ ID NO:5 or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO:5 while the functional properties of the enveloped virus comprising a glycoprotein GP encoding an amino acid sequence as shown in SEQ ID NO:5 are maintained.

It is to be understood that a recombinant enveloped virus may encode in its genome further cargos, such as tumor antigens, further chemokines, cytokines, or other immunomodulatory elements.

In a preferred embodiment the RNA genome of the vesicular stomatitis virus comprises or consists of a sequence as shown in SEQ ID NO: 6 or 7 or 8. Furthermore, the RNA genome of the vesicular stomatitis virus may also consist of or comprise those sequences, wherein nucleic acids of the RNA genome are exchanged according to the degeneration of the genetic code, without leading to an alteration of respective amino acid sequence. In a further preferred embodiment, the RNA genome of the vesicular stomatitis virus comprises or consists of a coding sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6 or 7 or 8.

### Definitions

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions×100). In some embodiments, the two sequences that are compared are the same length after gaps are introduced within the sequences, as appropriate (e.g., excluding additional sequence extending beyond the sequences being compared).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup-2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

The general embodiments “comprising” or “comprise” as used herein encompass the more specific embodiment “consisting of”. Furthermore, singular and plural forms are not used in a limiting way. As used herein, the singular forms “a”, “an” and “the” designate both the singular and the plural, unless expressly stated to designate the singular only.

A “pharmaceutical formulation” or “formulation” refers to the process but also the product of a process in which an active drug or agent is combined with chemical substances to produce a final medicinal or drug product, the final formulation therefore refers to medicinal products such as liquids, frozen liquids, dry preparations or compositions. Therefore, in one embodiment, a pharmaceutical formulation is a pharmaceutical composition.

A “pharmaceutical composition” refers in this context to a liquid, frozen liquid or dry preparation, which is in such form as to permit the biological activity of the active ingredient(s) to be unequivocally effective, and which contains no additional components, which are significantly toxic to the subjects to which the composition would be administered. Such compositions are sterile.

It will be understood that the different formulations described here can be provided as liquids, frozen liquids, and/or dry formulations. Hence, if not specified otherwise, the general term formulation or pharmaceutical formulation or composition or pharmaceutical composition encompasses all of liquids, frozen liquids, and/or dry compositions/formulations.

A “dry preparation” or a “dry pharmaceutical composition” is prepared by removing the liquid of a preparation containing the enveloped virus that has been formulated in a liquid solution. The removal of the liquid can be accomplished by e.g., evaporation, such as by the application of the liquid solution to a solid substrate and evaporation of the liquid and/or by sublimation such as by lyophilization (freeze-drying). The dry preparations/dry pharmaceutical compositions of the present invention are stored as dried formulations generally with 0.1% to 10.0% (w/w) residual moisture content (RMC). The dry preparations/dry pharmaceutical compositions can be reconstituted prior to administration, e.g., in an aqueous solutions, such as but not limited to sterile water, saline solutions, buffer solutions, aqueous dextrose or glycerol solutions, and the like. In particular embodiments the dry preparations/dry pharmaceutical compositions of the present invention are stored as dried formulations comprising 0.1% to 5% (w/w) residual moisture content. In more particular embodiments the dry preparations/dry pharmaceutical compositions of the present invention are stored as dried formulations comprising 0.25% to 2.5% (w/w) residual moisture content.

It will be understood that a dry pharmaceutical composition according to the invention will be originally prepared as a liquid pharmaceutical composition. Therefore, it will be further understood that if concentration ranges and/or pH ranges are given for a dry pharmaceutical composition, said concentration ranges and/or pH ranges refer to the originally prepared liquid pharmaceutical composition before it is dried and/or to the liquid pharmaceutical composition obtained after reconstitution of the dry pharmaceutical composition with an aqueous solution, e.g., with water.

It follows that in one embodiment a pharmaceutical composition according to the invention is a liquid pharmaceutical composition comprising an aqueous solution, preferably water, and any of the (dry) pharmaceutical compositions as described herein.

It further follows that in another embodiment, a pharmaceutical composition according to the invention is a liquid pharmaceutical composition obtained by reconstituting a dry pharmaceutical composition according to the invention in an aqueous solution, preferably water.

As used herein, the term “water” refers to water for injection.

The “pharmaceutically acceptable” excipients (vehicles, additives) are those, which are suitable for parenteral administration to a subject.

In one embodiment, the pharmaceutical formulation of the present invention is stable.

“Stability” refers to chemical stability and physical stability and can be evaluated qualitatively and/or quantitatively using various analytical techniques that are described in the art and are reviewed in for example in: Moving oncolytic viruses into the clinic: clinical-grade production, purification, and characterization of diverse oncolytic viruses. Mol Ther Methods Clin Dev. 2016 Apr. 6; 3:16018. doi: 10.1038/mtm.2016.18. PMID: 27088104; PMCID: PMC4822647. Those methods include the evaluation of aggregate and particle formation (for example using high-performance size exclusion chromatography (HP-SEC), by measuring turbidity, sub-visible particles by light obscuration (LO) of or microflow imaging (MFI=flow imaging microscopy (FIM)), dynamic imaging analysis (DIA), and/or by visual inspection of color and clarity); by assessing charge heterogeneity using cation exchange chromatography (CEX), or capillary isoelectric focusing; mass spectrometric analysis; capillary gel electrophoresis (CGE) analysis; peptide map (for example tryptic or Lys-C digest) analysis; and evaluating biological activity (infectivity); etc. In order to measure stability, a sample of the formulation of the invention may be tested in a stability study, wherein a sample is exposed for a selected time period to a stress condition followed by quantitative and qualitative analysis of the chemical and physical stability and infectivity using an adequate analytical technique.

Accordingly, stability can be measured at a selected temperature for a selected time period for instance by storing a sample at different temperatures such as −80° C., −20°, 2-8° C., room temperature (RT), 25° C., or 30° C. for up to 12 months and by using for instance HP-SEC, CEX, MFI, LO, CGE, or infectivity for qualitative and quantitative analysis.

According to the above, a “stable formulation” is one in which the formulation containing the enveloped virus is physically and chemically stable and/or retains its biological activity upon storage.

“Physical stability” refers substantially in context of the invention to an enveloped virus having little or no signs of aggregation, precipitation, and/or loss of infectivity. Methods to access the physical stability are for example SEC, LO, MFI, or dynamic imaging analysis (DIA) and visual inspections. For SEC, extensive peak broadening or tailing might be considered as a significantly difference in the context of the invention under the tested conditions depending on the column used, operating pressure, flow rate of the buffer. Using MFI, a significant increase in the number of particles, in particular particles larger than 2 μm and/or 10 μm might be considered as a significantly difference especially if particle numbers exceed compendial limits. Methods to access the infectivity are the median tissue culture infective dose, i.e., the quantity of a cytopathogenic agent, which will produce a cytopathic effect in 50% of the seeded cells.

The term “stress” or “stress condition” in context of the invention refers to e.g., mechanical stress, thermal stress, light stress, or stress resulting from freezing and thawing and particularly as shown in the Example section. Methods and conditions to simulate mechanical stress, thermal stress, light stress, or stress resulting from freezing and thawing are diverse and known to those skilled in the art. Mechanical stress may be for example shaking with 300 rpm at room temperature for up to 48 hours or vigilantly shaking the virus-containing vial by hand. Thermal stress refers for example to the storage at decreased or increased temperatures for an amount of time, in one example samples may be stored at a temperature of 5° C., 25° C., or 30° C., wherein for instance 25° C. and 30° C. refer to an accelerated stress condition. Light stress might be for example storing the samples at a light intensity of about 1100 lux for 5 days at various temperatures. Samples might be exposed to stress from freezing and thawing by exposing the samples to several cycles of freezing, e.g., at a temperature of −80° C. for 24 h and thawing at RT for 2 h, wherein the temperature cycles are repeated 3-5 times.

The term “substantially free of chloride” has the meaning that no source of chloride ions has been added to the pharmaceutical composition, preferably no source of chloride ions has been exogenously added to the pharmaceutical composition. More preferably, the pharmaceutical composition is free of chloride ions.

### Buffers

As used herein “buffer” refers to a buffered solution that resists changes in pH by the action of its acid-base conjugate components. The “pH” herein refers to the acidity or basicity of the composition at room temperature. Standard methods to measure the pH of a composition are known to the skilled in the art. Typically, measuring pH consists of calibrating the instrument, placing the electrodes in a well-mixed sample, and then reading the pH directly from the pH meter.

In various aspects, the pharmaceutical composition may comprise a buffer. The exemplary buffers of the present invention include acetate, citrate, histidine, succinate, HEPES, tartrate, phosphate, citrate/phosphate, lactate, and Tris.

### Sugars

In various aspects, the pharmaceutical composition may comprise a sugar or a combination of several sugars. The exemplary sugars of the present invention include dextrose, fructose, galactose, glucose, raffinose, trehalose, or sucrose.

### Sugar Alcohols

In various aspects, the pharmaceutical composition may comprise a sugar alcohol or a combination of several sugar alcohols. The exemplary sugar alcohols of the present invention include mannitol, sorbitol, xylitol, maltitol, maltitol symp, lactitol, inositol, glycerol erythritol, isomalt, or hydrogenated starch hydroxylate.

### Poloxamer

As used herein, “EO-PO block copolymer” means a copolymer consisting of blocks of poly(ethylene oxide) and poly(propylene oxide).

As used herein, “Pluronic” means EO-PO block copolymers in the EOₓ-PO_{y}-EO_{z} configuration. This configuration is also referred to as “poloxamer”.

For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g., P188=poloxamer with a polyoxypropylene molecular mass of 7680-9510 g/mol and a 20% polyoxyethylene content). For poloxamer 188 the PPO chain contains a unit number ranging from 25 to 30, and each PEO block is composed of 75 to 85 EO units in average (L. Bollenbach, J. Buske, K. Mader, P. Garidel, International Journal of Pharmaceutics, Volume 620, 2022).

As used herein a “protein agent” is albumin, gelatin, serum albumin, recombinant albumin, bovine serum albumin, porcine serum albumin, human serum albumin, recombinant human albumin, preferably human serum albumin (HSA) or recombinant human albumin (rHA).

In some embodiments, the pharmaceutical composition comprises human serum albumin (HSA), preferably, recombinant human albumin (rHA). HSA is the most abundant protein found in human blood plasma. As used herein the term “recombinant” in the context of “HA” means that the rHA is a genetically engineered product or made by recombinant production methods. A rHA is not derived from (isolated or purified from) a natural product (e.g., human plasma) but may be produced e.g., via genetically engineered cells to produce the rHA, although other methods to obtain rHA may be used equally by the skilled artisan.

All of the following tables should be read in the way that the formulation comprises or consists of the components in the indicated concentration ranges. Optional components may or may not be part of the formulation. If the terms “at least one” or “one or more” are used in conjunction with a single concentration range then said concentration range is to be understood to apply individually for each component, e.g., if two amino acids are present and only one concentration range from 1 to 300 mM is given, then both amino acids individually have a concentration range from 1 to 300 mM. When the term “one or more” is used in conjunction with a total concentration range, then said total concentration range is the total concentration of said components in the formulation.

TABLE 1
Typical concentration ranges of the components of the formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| (optionally) A buffer | 1 to 100 mM |
| (optionally) At least one sugar | 1 to 500 mM |
| One or more amino acids | 1 to 300 mM |
| (optionally) One or more sugar alcohol | 1 to 200 mM |
| A poly(ethylene oxide) and poly(propylene | 0.01 g/L to 50 g/L |
| oxide) block copolymer, preferably | |
| poloxamer 188 | |
| and/or | |
| a protein agent, preferably human serum | 0.1 g/L to 50 g/L |
| albumin or recombinant human albumin | |

TABLE 1a
Typical concentration ranges of the components of the formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| (optionally) A buffer, selected from the group | 1 to 100 mM |
| consisting of: Acetate, citrate, histidine, | |
| succinate, HEPES, tartrate, phosphate, citrate/ | |
| phosphate, lactate, and Tris | |
| (optionally) At least one sugar, selected from | 1 to 500 mM |
| the group consisting of: | |
| Dextrose, fructose, galactose, glucose, raffinose, | |
| trehalose, and sucrose | |
| One or more amino acids, selected from the group | 1 to 300 mM |
| consisting of: | |
| Arginine, alanine, phenylalanine, glycine, | |
| glutamine, glutamic acid, methionine, and lysine | |
| (optionally) One or more sugar alcohol, selected | 1 to 200 mM |
| from the group consisting of: | |
| Mannitol, sorbitol, xylitol, maltitol, maltitol | |
| symp, lactitol, inositol, glycerol erythritol, | |
| isomalt, and hydrogenated starch hydroxylate | |
| A poly(ethylene oxide) and poly(propylene oxide) | 0.01 g/L to 50 g/L |
| block copolymer, preferably poloxamer 188 | |
| and/or | |
| a protein agent: albumin, gelatin, preferably | 0.1 g/L to 50 g/L |
| human serum albumin or recombinant human | |
| albumin | |

In the following formulations according to the invention are shown. Preferably, said formulations are provided as dry formulations. Dry formulations can be generated from a liquid formulation, wherein said liquid formulation before it is dried will comprise or consist of the mentioned components and has the described concentration ranges. Reconstitution of the dry formulations with an appropriate volume of an aqueous solution, such as water, will then again result in a liquid formulation having the below described components and concentration ranges.

TABLE 3
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A buffer | 1 to 100 mM |
| (optionally) At least one sugar | 1 to 500 mM |
| One or more sugar alcohol | 1 to 200 mM |
| One or more amino acid | each in 1 to 300 mM |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3a
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (optionally) At least one sugar | 1 to 500 mM |
| One or more sugar alcohol | 1 to 200 mM |
| One or more amino acid | each in 1 to 300 mM |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3b
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A buffer | 1 to 100 mM |
| (optionally) At least one sugar | 1 to 500 mM |
| One or more sugar alcohol, selected from the | 1 to 200 mM |
| group consisting of: mannitol, sorbitol, xylitol, | |
| maltitol, maltitol symp, lactitol, inositol, | |
| glycerol erythritol, isomalt, and hydrogenated | |
| starch hydroxylate | |
| One or more amino acid | each in 1 to 300 mM |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3c
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A buffer | 1 to 100 mM |
| (optionally) At least one sugar | 1 to 500 mM |
| One or more sugar alcohol | 1 to 200 mM |
| One or more amino acids, selected from the | each in 1 to 300 mM |
| group consisting of: arginine, alanine, | |
| phenylalanine, glycine, glutamine, glutamic | |
| acid, methionine, and lysine | |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3d
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A buffer, selected from the group consisting | 1 to 100 mM |
| of: acetate, citrate, histidine, succinate, | |
| HEPES, tartrate, phosphate, citrate/phosphate, | |
| lactate, and Tris | |
| (optionally) At least one sugar | 1 to 500 mM |
| One or more sugar alcohol, selected from the | 1 to 200 mM |
| group consisting of: mannitol, sorbitol, xylitol, | |
| maltitol, maltitol symp, lactitol, inositol, glycerol | |
| erythritol, isomalt, and hydrogenated starch | |
| hydroxylate | |
| One or more amino acid | each in 1 to 300 mM |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3e
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A buffer, selected from the group consisting | 1 to 100 mM |
| of: acetate, citrate, histidine, succinate, | |
| HEPES, tartrate, phosphate, citrate/phosphate, | |
| lactate, or Tris | |
| (optionally) At least one sugar | 1 to 500 mM |
| One or more sugar alcohol, selected from the | 1 to 200 mM |
| group consisting of: mannitol, sorbitol, xylitol, | |
| maltitol, maltitol symp, lactitol, inositol, glycerol | |
| erythritol, isomalt, and hydrogenated starch | |
| hydroxylate | |
| One or more amino acid, selected from the group | each in 1 to 300 mM |
| consisting of: arginine, alanine, phenylalanine, | |
| glycine, glutamine, glutamic acid, methionine, | |
| and lysine | |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3f
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more | 1 × 10¹² TCID50/mL |
| preferably VSV-GP | |
| A buffer, selected from the group consisting | 1 to 100 mM |
| of: acetate, citrate, histidine, succinate, | |
| HEPES, tartrate, phosphate, citrate/phosphate, | |
| lactate, and Tris | |
| (Optionally) At least one sugar, selected from | 1 to 500 mM |
| the group consisting of: dextrose, fructose, | |
| galactose, glucose, raffinose, trehalose, | |
| and sucrose | |
| One or more sugar alcohols, selected from the | 1 to 200 mM |
| group consisting of: mannitol, sorbitol, xylitol, | |
| maltitol, maltitol symp, lactitol, inositol, | |
| glycerol erythritol, isomalt, and hydrogenated | |
| starch hydroxylate | |
| One or more amino acids, selected from the group | 1 to 300 mM |
| consisting of: arginine, alanine, phenylalanine, | |
| glycine, glutamine, glutamic acid, methionine, | |
| and lysine | |
| A poly(ethylene oxide) and poly(propylene oxide) | 0.01 g/L to 50 g/L |
| block copolymer, preferably poloxamer 188 | |
| and/or | |
| a protein agent, preferably human serum albumin | 0.1 g/L to 50 g/L |
| or recombinant human albumin | |

TABLE 3g
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more | 1 × 10¹² TCID50/mL |
| preferably VSV-GP | |
| A Tris buffer | 1 to 100 mM |
| (Optionally) At least one sugar, selected | 1 to 500 mM |
| from the group consisting of: dextrose, fructose, | |
| galactose, glucose, raffinose, trehalose, | |
| and sucrose | |
| One or more sugar alcohol, selected from the | 1 to 200 mM |
| group consisting of: mannitol, sorbitol, xylitol, | |
| maltitol, maltitol symp, lactitol, inositol, | |
| glycerol erythritol, isomalt, and hydrogenated | |
| starch hydroxylate | |
| One or more amino acid, selected from the group | 1 to 300 mM |
| consisting of: arginine, alanine, phenylalanine, | |
| glycine, glutamine, glutamic acid, methionine, | |
| and lysine | |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3h
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more | 1 × 10¹² TCID50/mL |
| preferably VSV-GP | |
| A Tris buffer | 1 to 100 mM |
| (Optionally) At least one sugar, selected | 1 to 500 mM |
| from the group consisting of: dextrose, | |
| fructose, galactose, glucose, raffinose, | |
| trehalose, and sucrose | |
| Mannitol and/or sorbitol as sugar alcohol | 1 to 200 mM |
| One or more amino acid, selected from the group | 1 to 300 mM |
| consisting of: arginine, alanine, phenylalanine, | |
| glycine, glutamine, glutamic acid, methionine, | |
| and lysine | |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3i
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more | 1 × 10¹² TCID50/mL |
| preferably VSV-GP | |
| A Tris buffer | 1 to 100 mM |
| (Optionally) At least one sugar, selected | 1 to 500 mM |
| from the group consisting of: dextrose, | |
| fructose, galactose, glucose, raffinose, trehalose, | |
| and sucrose | |
| Mannitol and/or sorbitol as sugar alcohol | 1 to 200 mM |
| Glutamic acid and/or arginine as amino acid | 1 to 300 mM |
| A poly(ethylene oxide) and poly(propylene oxide) | 0.01 g/L to 50 g/L |
| block copolymer, preferably poloxamer 188 | |
| and/or | |
| a protein agent, preferably human serum | 0.1 g/L to 50 g/L |
| albumin or recombinant human albumin | |

TABLE 3j
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more | 1 × 10¹² TCID50/mL |
| preferably VSV-GP | |
| A Tris buffer | 1 to 100 mM |
| (Optionally) At least one sugar, selected | 1 to 500 mM |
| from the group consisting of: dextrose, | |
| fructose, galactose, glucose, raffinose, | |
| trehalose, and sucrose | |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and/or | |
| recombinant human albumin | 0.1 g/L to 50 g/L |

TABLE 3k
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more | 1 × 10¹² TCID50/mL |
| preferably VSV-GP | |
| A Tris buffer | 1 to 100 mM |
| (Optionally) At least one sugar, selected | 1 to 500 mM |
| from the group consisting of: dextrose, fructose, | |
| galactose, glucose, raffinose, trehalose, | |
| and sucrose | |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3l
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Tris buffer | 1 to 100 mM |
| Trehalose as a sugar | 1 to 500 mM |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3m
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) At least one sugar, selected from the | 1 to 500 mM |
| group consisting of: dextrose, fructose, galactose, | |
| glucose, raffinose, trehalose, and sucrose | |
| One or more sugar alcohol, selected from the | 1 to 200 mM |
| group consisting of: mannitol, sorbitol, xylitol, | |
| maltitol, maltitol symp, lactitol, inositol, glycerol | |
| erythritol, isomalt, and hydrogenated starch | |
| hydroxylate | |
| One or more amino acid, selected from the group | 1 to 300 mM |
| consisting of: arginine, alanine, phenylalanine, | |
| glycine, glutamine, glutamic acid, methionine, | |
| and lysine | |
| poloxamer 188 | 0.01 g/L to 50 g/L |
| and/or | |
| recombinant human albumin | 0.1 g/L to 50 g/L |

TABLE 3n
Typical concentration ranges of the components of a formulation,
preferably a dry formulation such as a lyophilized formulation

| | Concentration |
|---|---|
| Component | Range |
| An enveloped virus, preferably a vesiculovirus or | 1 × 10⁵ TCID50/mL to |
| a vesicular stomatitis virus, more preferably | 1 × 10¹² TCID50/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) At least one sugar, selected from the | 1 to 500 mM |
| group consisting of: dextrose, fructose, galactose, | |
| glucose, raffinose, trehalose, and sucrose | |
| One or more sugar alcohol, selected from the | 1 to 200 mM |
| group consisting of: mannitol, sorbitol, xylitol, | |
| maltitol, maltitol symp, lactitol, inositol, glycerol | |
| erythritol, isomalt, and hydrogenated starch | |
| hydroxylate | |
| One or more amino acid, selected from the group | 1 to 300 mM |
| consisting of: arginine, alanine, phenylalanine, | |
| glycine, glutamine, glutamic acid, methionine, | |
| and lysine | |
| poloxamer 188 | 0.01 g/L to 50 g/L |
| and | |
| recombinant human albumin | 0.1 g/L to 50 g/L |

TABLE 3o
Typical concentration ranges of the components of a formulation,
preferably a dry formulation such as a lyophilized formulation

| | Concentration |
|---|---|
| Component | Range |
| An enveloped virus, preferably a vesiculovirus or | 1 × 10⁵ TCID50/mL to |
| a vesicularstomatitis virus, more preferably | 1 × 10¹² TCID50/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) At least one sugar, selected from the | 1 to 500 mM |
| group consisting of: dextrose, fructose, galactose, | |
| glucose, raffinose, trehalose, and sucrose | |
| One or more sugar alcohol, selected from the | 1 to 200 mM |
| group consisting of: mannitol, sorbitol, xylitol, | |
| maltitol, maltitol symp, lactitol, inositol, | |
| glycerol erythritol, isomalt, and hydrogenated | |
| starch hydroxylate | |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3p
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) At least one sugar, selected from the | 1 to 500 mM |
| group consisting of: dextrose, fructose, galactose, | |
| glucose, raffinose, trehalose, and sucrose | |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3q
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Tris buffer | 1 to 100 mM |
| (Optionally) At least one sugar, selected from the | 1 to 500 mM |
| group consisting of: dextrose, fructose, galactose, | |
| glucose, raffinose, trehalose, and sucrose | |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3r
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Tris buffer | 1 to 100 mM |
| At least one sugar, selected from the group | 1 to 500 mM |
| consisting of: dextrose, fructose, galactose, | |
| glucose, raffinose, trehalose, and sucrose | |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3s
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Tris buffer | 1 to 100 mM |
| Trehalose as a sugar | 1 to 500 mM |
| Mannitol and/or sorbitol as sugar alcohol | 1 to 200 mM |
| Glutamic acid and/or arginine as amino acid | 1 to 300 mM |
| poloxamer 188 | 0.01 g/L to 50 g/L |
| and | |
| recombinant human albumin | 0.1 g/L to 50 g/L |

TABLE 3t
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID50/mL to |
| or a vesicular stomatitis virus, more preferably | 1 × 10¹² TCID50/mL |
| VSV-GP | |
| A Tris buffer | 5 to 50 mM |
| Trehalose as a sugar | 50 to 250 mM |
| Mannitol and/or sorbitol as sugar alcohol | 5 to 100 mM |
| Glutamic acid and/or arginine as amino acid | 5 to 100 mM |
| poloxamer 188 | 0.05 g/L to 10 g/L |
| and | |
| recombinant human albumin | 2.5 g/L to 10 g/L |

In another embodiment, the formulation, preferably the dry formulation comprises an enveloped virus, about 10-50 mM Tris, about 100-500 mM trehalose, about 0-50 mM mannitol, about 50-100 mM sorbitol, about 5-25 mM glutamic acid, about 3-10 g/L rHA, and about 1.5-5 g/L poloxamer 188, a pH between 7.0 and 8.0, preferably about pH 7.4.

In another embodiment, the formulation, preferably the dry formulation comprises an enveloped virus, about 10 mM Tris, about 200 mM trehalose, about 50 mM sorbitol, about 20 mM glutamic acid, about 5 g/L rHA, and about 1.5-5 g/L poloxamer 188, a pH between 7.0 and 8.0, preferably about pH 7.4.

In another embodiment, the formulation, preferably the dry formulation comprises an enveloped virus, about 8-12 mM Tris, about 180-220 mM trehalose, about 40-60 mM sorbitol, about 15-25 mM glutamic acid, about 4-6 g/L rHA, and about 1.5-5 g/L poloxamer 188, a pH between 7.0 and 8.0, preferably about pH 7.4.

In another embodiment, the formulation, preferably the dry formulation comprises an enveloped virus, about 10 mM Tris, about 200 mM trehalose, about 50 mM sorbitol, about 50 mM mannitol, about 20 mM glutamic acid, about 5 g/L rHA, and about 2 g/L poloxamer 188, a pH between 7.0 and 8.0, preferably about pH 7.4.

In another embodiment, the formulation, preferably the dry formulation comprises an enveloped virus, about 8-12 mM Tris, about 180-220 mM trehalose, about 40-60 mM sorbitol, about 40-60 mM mannitol, about 15-25 mM glutamic acid, about 4-6 g/L rHA, and about 1.5-2.5 g/L poloxamer 188, a pH between 7.0 and 8.0, preferably about pH 7.4.

In another embodiment, the formulation, preferably the dry formulation comprises an enveloped virus, about 20 mM Tris, about 150 mM sucrose, about 50 mM mannitol, about 50 mM sorbitol, about 20 mM glutamic acid, about 5 g/L rHA, a pH between 7.0 and 8.0, preferably about pH 7.4.

In another embodiment, the formulation, preferably the dry formulation comprises an enveloped virus, about 15-25 mM Tris, about 140-160 mM sucrose, about 40-60 mM mannitol, about 40-60 mM sorbitol, about 15-25 mM glutamic acid, about 4-6 g/L rHA, a pH between 7.0 and 8.0, preferably about pH 7.4.

In another embodiment, the formulation, preferably the dry formulation comprises an enveloped virus, about 10 mM Tris, about 200 mM trehalose, about 50 mM mannitol, about 50 mM sorbitol, about 20 mM glutamic acid, about 10 g/L rHA, and about 0.05 g/L poloxamer 188, a pH between 7.0 and 8.0, preferably about pH 7.4.

In another embodiment, the formulation, preferably the dry formulation comprises an enveloped virus, about 8-12 mM Tris, about 180-220 mM trehalose, about 40-60 mM mannitol, about 40-60 mM sorbitol, about 15-25 mM glutamic acid, about 8-12 g/L rHA, and about 0.05-1.5 g/L poloxamer 188, a pH between 7.0 and 8.0, preferably about pH 7.4.

In another embodiment, the formulation, preferably the dry formulation comprises an enveloped virus, about 10 mM Tris, about 200 mM trehalose, about 50 mM mannitol, about 50 mM sorbitol, about 20 mM glutamic acid, about 10 g/L rHA, and about 2.5 g/L poloxamer 188, a pH between 7.0 and 8.0, preferably about pH 7.4.

In another embodiment, the formulation, preferably the dry formulation comprises an enveloped virus, about 8-12 mM Tris, about 180-220 mM trehalose, about 40-60 mM mannitol, about 40-60 mM sorbitol, about 15-25 mM glutamic acid, about 8-12 g/L rHA, and about 1.5-3.5 g/L poloxamer 188, a pH between 7.0 and 8.0, preferably about pH 7.4.

In another embodiment, the formulation, preferably the dry formulation comprises an enveloped virus, about 10 mM Tris, about 200 mM trehalose, about 50 mM mannitol, about 50 mM sorbitol, about 20 mM glutamic acid, about 10 g/L rHA, and about 5 g/L poloxamer 188, a pH between 7.0 and 8.0, preferably about pH 7.4.

In another embodiment, the formulation, preferably the dry formulation comprises an enveloped virus, about 8-12 mM Tris, about 180-220 mM trehalose, about 40-60 mM mannitol, about 40-60 mM sorbitol, about 15-25 mM glutamic acid, about 8-12 g/L rHA, and about 2.5-6 g/L poloxamer 188, a pH between 7.0 and 8.0, preferably about pH 7.4.

In another embodiment, the formulation, preferably the dry formulation comprises an enveloped virus, about 10 mM Tris, about 200 mM trehalose, about 20.7-34.6 mM mannitol, about 49-49.6 mM sorbitol, about 20 mM glutamic acid, about 3.4-4.7 g/L rHA, and about 1.4 g/L poloxamer 188, a pH between 7.0 and 8.0, preferably about pH 7.4.

In any of the above-mentioned formulations, preferably dry formulations embodiments the buffer concentration can be in any concentration range between 1 to 100 mM, such as 1 to 90 mM, 1 to 80 mM, 1 to 70 mM, 1 to 60 mM, or 1 to 50 mM. Preferably, the buffer concentration is between 5 mM and 50 mM, 5 mM and 40 mM, 5 mM and 30 mM, or 5 and 20 mM. Preferably, the dry formulation comprises a Tris buffer and more preferably a Tris buffer in a concentration between 5 and 50 mM.

In any of the above-mentioned formulations, preferably dry formulation embodiments the sugar concentration can be in any concentration range between 1 to 500 mM, such as 1 to 490 mM, 1 to 480 mM, 1 to 470 mM, 1 to 460 mM, 1 to 450 mM, 1 to 440 mM, 1 to 430 mM, 1 to 420 mM, 1 to 410 mM, 1 to 400 mM, 1 to 390 mM, 1 to 380 mM, 1 to 370 mM, 1 to 360 mM, 1 to 350 mM, 1 to 340 mM, 1 to 330 mM, 1 to 320 mM, 1 to 310 mM, 1 to 300 mM, 1 to 290 mM, 1 to 280 mM, 1 to 270 mM, 1 to 260 mM, 1 to 250 mM, 1 to 240 mM, 1 to 230 mM, 1 to 220 mM, 1 to 210 mM, or 1 to 200 mM. Preferably, the sugar concentration is between 50 mM and 300 mM, 50 mM and 250 mM, or 50 mM and 200 mM. Preferably, the formulation comprises a sugar, such as dextrose, fructose, galactose, glucose, raffinose, trehalose, or sucrose. More preferably the sugar is trehalose and in a concentration between 50 and 250 mM.

In any of the above-mentioned formulations, preferably dry formulation embodiments the amino acid concentration can be in any concentration range between 1 to 300 mM such as 1 to 290 mM, 1 to 280 mM, 1 to 270 mM, 1 to 260 mM, 1 to 250 mM, 1 to 240 mM, 1 to 230 mM, 1 to 220 mM, 1 to 210 mM, 1 to 200 mM, 1 to 190 mM, 1 to 180 mM, 1 to 170 mM, 1 to 160 mM, 1 to 150 mM, 1 to 140 mM, 1 to 130 mM, 1 to 120 mM, 1 to 110 mM, 1 to 100 mM, 1 to 90 mM, 1 to 80 mM, 1 to 70 mM, 1 to 60 mM, 1 to 50 mM, 1 to 40 mM, 1 to 30 mM, 1 to 20 mM, or 1 to 10 mM. Preferably, the amino acid concentration is between 5 mM and 100 mM, 5 mM and 90 mM, 5 mM or 80 mM, 5 mM and 70 mM, 5 mM and 60 mM, or 5 mM and 50 mM. Preferably, dry formulation comprises an amino acid, such as arginine, alanine, phenylalanine, glycine, glutamine, glutamic acid, methionine, or lysine. More preferably the amino acid is glutamic acid. Most preferred is glutamic acid in a concentration between 5 and 100 mM or 5 and 50 mM, or 5 and 20 mM.

In any of the above-mentioned formulations, preferably dry formulation embodiments the sugar alcohol concentration can be in any concentration range between 1 to 200 mM such as 1 to 190 mM, 1 to 180 mM, 1 to 170 mM, 1 to 160 mM, 1 to 150 mM, 1 to 140 mM, 1 to 130 mM, 1 to 120 mM, 1 to 110 mM, or 1 to 100 mM. Preferably, the sugar alcohol concentration is between 5 and 100 mM, 10 and 100 mM, 10 or 80 mM, 10 and 70 mM, 10 and 60 mM, or 10 and 50 mM. Preferably, the dry formulation comprises one or more sugar alcohol such as mannitol, sorbitol, xylitol, maltitol, maltitol symp, lactitol, inositol, glycerol erythritol, isomalt, or hydrogenated starch hydroxylate. More preferably the sugar alcohol is mannitol and/or sorbitol, more preferred a combination of mannitol and sorbitol. Most preferred is a combination of mannitol and sorbitol each independently in a concentration between 5 and 100 mM. In any of the above mentioned formulations, preferably dry formulation embodiments the poloxamer concentration can be in any concentration range between 0.01 g/L to 50 g/L, such as 0.02 g/L to 50 g/L, 0.03 g/L to 50 g/L, 0.04 g/L to 50 g/L, 0.05 g/L to 50 g/L, 0.05 g/L to 49 g/L, 0.05 g/L to 48 g/L, 0.05 g/L to 47 g/L, 0.05 g/L to 46 g/L, 0.05 g/L to 45 g/L, 0.05 g/L to 44 g/L, 0.05 g/L to 43 g/L, 0.05 g/L to 42 g/L, 0.05 g/L to 41 g/L, 0.05 g/L to 40 g/L, 0.05 g/L to 39 g/L, 0.05 g/L to 38 g/L, 0.05 g/L to 37 g/L, 0.05 g/L to 36 g/L, 0.05 g/L to 35 g/L, 0.05 g/L to 34 g/L, 0.05 g/L to 33 g/L, 0.05 g/L to 32 g/L, 0.05 g/L to 31 g/L, 0.05 g/L to 30 g/L, 0.05 g/L to 29 g/L, 0.05 g/L to 28 g/L, 0.05 g/L to 27 g/L, 0.05 g/L to 26 g/L, 0.05 g/L to 25 g/L, 0.05 g/L to 24 g/L, 0.05 g/L to 23 g/L, 0.05 g/L to 22 g/L, 0.05 g/L to 21 g/L, 0.05 g/L to 19 g/L, 0.05 g/L to 18 g/L, 0.05 g/L to 17 g/L, 0.05 g/L to 16 g/L, 0.05 g/L to 15 g/L, 0.05 g/L to 14 g/L, 0.05 g/L to 13 g/L, 0.05 g/L to 12 g/L, 0.05 g/L to 11 g/L, or 0.05 g/L to 40 g/L. More preferred ranges include 0.05 g/L to 15 g/L, 0.05 g/L to 14 g/L, 0.05 g/L to 13 g/L, 0.05 g/L to 12 g/L, 0.05 g/L to 11 g/L, or 0.05 g/L to 10 g/L. Preferably, the formulation comprises a pharmaceutically acceptable poloxamer. More preferably the poloxamer is poloxamer 188. Most preferred is poloxamer 188 in a concentration between 0.05 g/L to 20 g/L, 0.05 g/L to 15 g/L, 0.05 g/L to 10 g/L, 0.5 g/L to 10 g/L, or 1 g/L to 10 g/L.

In any of the above mentioned formulations, preferably dry formulation embodiments the protein agent concentration can be in any concentration range between 0.1 g/L to 50 g/L, such as 1 g/L to 50 g/L, 1.1 g/L to 50 g/L, 1.2 g/L to 50 g/L, 1.3 g/L to 50 g/L, 1.4 g/L to 50 g/L, 1.5 g/L to 50 g/L, 1.6 g/L to 50 g/L, 1.7 g/L to 50 g/L, 1.8 g/L to 50 g/L, 1.9 g/L to 50 g/L, 2.0 g/L to 50 g/L, 2.1 g/L to 50 g/L, 2.2 g/L to 50 g/L, 2.3 g/L to 50 g/L, 2.4 g/L to 50 g/L, 2.5 g/L to 50 g/L. More preferred ranges include 1 g/L to 45 g/L, 1 g/L to 40 g/L, 1 g/L to 35 g/L, 1 g/L to 30 g/L, 1 g/L to 25 g/L, 1 g/L to 20 g/L, 1 g/L to 15 g/L, 1.5 g/L to 10 g/L, 2.0 g/L to 10 g/L, or 2.5 g/L to 10 g/L. Preferably, the formulation comprises recombinant human albumin. Most preferred is recombinant human albumin in a concentration between 1 g/L to 20 g/L, 1 g/L to 15 g/L, 1.5 g/L to 10 g/L, 2.0 g/L to 10 g/L, or 2.5 g/L to 10 g/L.

The pH of the above-mentioned formulations, preferably dry formulation embodiments is usually kept in a range between 5 to 9, or between 6 to 9, or between 6.5 to 8.5, or between 6.5 to 8.0, preferably between 7.0 and 8.0. The person skilled in the art will understand that the pH of the formulation refers to the pH of the formulation when in aqueous form.

In any of the above-mentioned formulations, preferably dry formulation embodiments the enveloped virus is preferably a vesiculovirus, more preferably a vesicular stomatitis virus and most preferred a vesicular stomatitis virus having the glycoprotein G replaced with the glycoprotein GP of the lymphocytic choriomeningitis virus (LCMV). In any of the above-mentioned formulation embodiments the virus concentration can be in any concentration range between 1×10⁵ TCID₅₀/mL to 1×10¹² TCID₅₀/mL or in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, or at least 1×10⁹ TCID₅₀/mL. Other ranges include between 1×10⁶ TCID₅₀/mL to 1×10¹² TCID₅₀/mL, between 1×10⁶ TCID₅₀/mL to 1×10¹¹ TCID₅₀/mL, and the like.

In the following further formulations according to the invention are shown.

Again, all of the following tables should be read in the way that the formulation comprises or consists of the components in the indicated concentration ranges. Optional components may or may not be part of the formulation. If the terms “at least one” or “one or more” are used in conjunction with a single concentration range then said concentration range is to be understood to apply individually for each component, e.g., if two amino acids are present and only one concentration range from 1 to 300 mM is given, then both amino acids individually have a concentration range from 1 to 300 mM. Additionally, if the term “one or more” is used in conjunction with a total concentration range, then said total concentration range is the total concentration of said one ore more components in the formulation.

TABLE 3
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| One or more sugar alcohol | total concentration of |
| | 1 mM to 1000 mM |
| | OR |
| | total concentration of at |
| | least 10 mM |
| (optionally) At least one of a buffer, a sugar, | Buffer 1 to 100 mM |
| or an amino acid | Sugar 1 to 500 mM |
| | Amino acid 1 to 300 mM |
| As poly(ethylene oxide) and poly(propylene | 0.05 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3a
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| One or more sugar alcohol, wherein the one or | total concentration of |
| more sugar alcohol is selected from the group | 1 mM to 1000 mM |
| consisting of: mannitol, sorbitol, xylitol, | OR |
| maltitol, maltitol symp, lactitol, inositol, | total concentration of at |
| glycerol erythritol, isomalt, and hydrogenated | least 10 mM |
| starch hydroxylate, preferably mannitol and/or | |
| sorbitol, more preferably a combination of | |
| mannitol and sorbitol, | |
| A Buffer selected from the group consisting | 1 to 100 mM |
| of: acetate, citrate, histidine, succinate, | |
| HEPES, tartrate, phosphate, citrate/phosphate, | |
| lactate, and Tris; preferably Tris | |
| As poly(ethylene oxide) and poly(propylene | 0.05 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3b
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| One or more sugar alcohols, wherein the one or | total concentration of |
| more sugar alcohol is selected from the group | 1 mM to 1000 mM |
| consisting of: mannitol, sorbitol, xylitol, | OR |
| maltitol, maltitol symp, lactitol, inositol, | total concentration of at |
| glycerol erythritol, isomalt, and hydrogenated | least 10 mM |
| starch hydroxylate, preferably mannitol and/or | |
| sorbitol, more preferably a combination of | |
| mannitol and sorbitol, | |
| A sugar, selected from the group consisting of: | 1 to 500 mM |
| dextrose,fructose, galactose, glucose, raffinose, | |
| trehalose, and sucrose | |
| As poly(ethylene oxide) and poly(propylene | 0.05 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3c
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| One or more sugar alcohol, wherein the one or | total concentration of |
| more sugar alcohol is selected from the group | 1 mM to 1000 mM |
| consisting of: mannitol, sorbitol, xylitol, | OR |
| maltitol, maltitol symp, lactitol, inositol, | total concentration of at |
| glycerol erythritol, isomalt, and hydrogenated | least 10 mM |
| starch hydroxylate, preferably mannitol and/or | |
| sorbitol, more preferably a combination of | |
| mannitol and sorbitol, | |
| An Amino acid selected from the group | 1 to 300 mM |
| consisting of: arginine, alanine, phenylalanine, | |
| glycine, glutamine, glutamic acid, methionine, | |
| and lysine | |
| As poly(ethylene oxide) and poly(propylene | 0.05 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3d
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| One or more sugar alcohol, wherein the one or | total concentration of |
| more sugar alcohol is selected from the group | 1 mM to 1000 mM |
| consisting of: mannitol, sorbitol, xylitol, | OR |
| maltitol, maltitol symp, lactitol, inositol, | total concentration of at |
| glycerol erythritol, isomalt, and hydrogenated | least 10 mM |
| starch hydroxylate, preferably mannitol and/or | |
| sorbitol, more preferably a combination of | |
| mannitol and sorbitol, | |
| A Buffer selected from the group consisting | 1 to 100 mM |
| of: acetate, citrate, histidine, succinate, | |
| HEPES, tartrate,phosphate, citrate/phosphate, | |
| lactate, or Tris; preferably Tris | |
| A sugar selected from the group consisting of: | 1 to 500 mM |
| dextrose, fructose, galactose, glucose, | |
| raffinose, trehalose, and sucrose | |
| As poly(ethylene oxide) and poly(propylene | 0.05 g/L to 50 g/L |
| boxide) lock copolymer: poloxamer 188 | |
| and/or | |
| As protein agent: human serum albumin or | 0.05 g/L to 50 g/L |
| recombinant human albumin | |

TABLE 3d
Typical concentration ranges of the components of a formulation,
preferably a dry formulation such as a lyophilized formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| One or more sugar alcohol, wherein the one or | total concentration of |
| more sugar alcohol is selected from the group | 1 mM to 1000 mM |
| consisting of: mannitol, sorbitol, xylitol, | OR |
| maltitol, maltitol symp, lactitol, inositol, | total concentration of at |
| glycerol erythritol, isomalt, and hydrogenated | least 10 mM |
| starch hydroxylate, preferably mannitol and/or | |
| sorbitol, more preferably a combination of | |
| mannitol and sorbitol, | |
| A Buffer selected from the group consisting | 1 to 100 mM |
| of: acetate, citrate, histidine, succinate, | |
| HEPES, tartrate, phosphate, citrate/phosphate, | |
| lactate, and Tris; preferably Tris | |
| An Amino acid selected from the group | 1 to 300 mM |
| consisting of: arginine, alanine, phenylalanine, | |
| glycine, glutamine, glutamic acid, methionine, | |
| and lysine | |
| As poly(ethylene oxide) and poly(propylene | 0.05 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3e
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of |
| preferably mannitol and/or sorbitol, more preferably a | at least 10 mM |
| combination of mannitol and sorbitol, | |
| A Buffer selected from the group consisting of: | 1 to 100 mM |
| acetate, citrate, histidine, succinate, HEPES, tartrate, | |
| phosphate, citrate/phosphate, lactate, and Tris; preferably Tris | |
| An Amino acid selected from the group consisting of: arginine, | 1 to 300 mM |
| alanine, phenylalanine, glycine, glutamine, glutamic acid, | |
| methionine, and lysine | |
| A sugar selected from the group consisting of: dextrose, | 1 to 500 mM |
| fructose, galactose, glucose, raffinose, trehalose, and sucrose | |
| As poly(ethylene oxide) and poly(propylene oxide) block | 0.05 g/L to 50 g/L |
| copolymer: poloxamer 188 | |
| and/or | |
| As protein agent: human serum albumin or recombinant human | 0.05 g/L to 50 g/L |
| albumin | |

TABLE 3f
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| An Amino acid selected from the group consisting of: arginine, | 1 to 300 mM |
| alanine, phenylalanine, glycine, glutamine, glutamic acid, | |
| methionine, and lysine | |
| A sugar selected from the group consisting of: dextrose, | 1 to 500 mM |
| fructose, galactose, glucose, raffinose, trehalose, and sucrose | |
| As poly(ethylene oxide) and poly(propylene oxide) block | 0.05 g/L to 50 g/L |
| copolymer: poloxamer 188 | |
| and/or | |
| As protein agent: human serum albumin or recombinant human | 0.05 g/L to 50 g/L |
| albumin | |

TABLE 3g
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| Tris buffer | 1 to 100 mM |
| An Amino acid selected from the group consisting of: arginine, | 1 to 300 mM |
| alanine, phenylalanine, glycine, glutamine, glutamic acid, | |
| methionine, and lysine | |
| A sugar selected from the group consisting of: dextrose, | 1 to 500 mM |
| fructose, galactose, glucose, raffinose, trehalose, and sucrose | |
| As poly(ethylene oxide) and poly(propylene oxide) block | 0.05 g/L to 10 g/L |
| copolymer: poloxamer 188 | |
| and/or | |
| As protein agent: human serum albumin or recombinant human | 0.05 g/L to 10 g/L |
| albumin | |

TABLE 3h
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| A Buffer selected from the group consisting of: | 1 to 100 mM |
| acetate, citrate, histidine, succinate, HEPES, tartrate, | |
| phosphate, citrate/phosphate, lactate, and Tris; preferably Tris | |
| Arginine and/or glutamic acid | 1 to 300 mM |
| A sugar selected from the group consisting of: dextrose, | 1 to 500 mM |
| fructose, galactose, glucose, raffinose, trehalose, and sucrose | |
| As poly(ethylene oxide) and poly(propylene oxide) block | 0.05 g/L to 10 g/L |
| copolymer: poloxamer 188 | |
| and/or | |
| As protein agent: human serum albumin or recombinant human | 0.05 g/L to 10 g/L |
| albumin | |

TABLE 3i
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| A Buffer selected from the group consisting of: | 1 to 100 mM |
| acetate, citrate, histidine, succinate, HEPES, tartrate, | |
| phosphate, citrate/phosphate, lactate, and Tris; preferably Tris | |
| An Amino acid selected from the group consisting of: arginine, | 1 to 300 mM |
| alanine, phenylalanine, glycine, glutamine, glutamic acid, | |
| methionine, and lysine | |
| Trehalose, or sucrose | 1 to 500 mM |
| As poly(ethylene oxide) and poly(propylene oxide) block | 0.05 g/L to 10 g/L |
| copolymer: poloxamer 188 | 0.05 g/L to 10 g/L |
| and/or | |
| As protein agent: human serum albumin or recombinant human | |
| albumin | |

TABLE 3j
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| Tris | 1 to 100 mM |
| Arginine and/or glutamic acid | 1 to 300 mM |
| A sugar selected from the group consisting of: dextrose, | 1 to 500 mM |
| fructose, galactose, glucose, raffinose, trehalose, and sucrose | |
| As poly(ethylene oxide) and poly(propylene oxide) block | 0.05 g/L to 10 g/L |
| copolymer: poloxamer 188 | |
| and/or | |
| As protein agent: human serum albumin or recombinant human | 0.05 g/L to 10 g/L |
| albumin | |

TABLE 3k
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| Tris buffer | 1 to 100 mM |
| An Amino acid selected from the group consisting of: arginine, | 1 to 300 mM |
| alanine, phenylalanine, glycine, glutamine, glutamic acid, | |
| methionine, and lysine | |
| Trehalose or sucrose | 1 to 500 mM |
| As poly(ethylene oxide) and poly(propylene oxide) block | 0.05 g/L to 10 g/L |
| copolymer: poloxamer 188 | |
| and/or | |
| As protein agent: human serum albumin or recombinant human | 0.05 g/L to 10 g/L |
| albumin | |

TABLE 3l
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| One or more sugar alcohol, wherein the one or more sugar | total concentration of |
| alcohol is selected from the group consisting of: mannitol, | 1 mM to 1000 mM |
| sorbitol, xylitol, maltitol, maltitol symp, lactitol, inositol, glycerol | OR |
| erythritol, isomalt, and hydrogenated starch hydroxylate, | total concentration of at |
| preferably mannitol and/or sorbitol, more preferably a | least 10 mM |
| combination of mannitol and sorbitol, | |
| A Buffer selected from the group consisting of: | 1 to 100 mM |
| acetate, citrate, histidine, succinate, HEPES, tartrate, | |
| phosphate, citrate/phosphate, lactate, and Tris; preferably Tris | |
| Arginine and/or glutamic acid | 1 to 300 mM |
| Trehalose or sucrose | 1 to 500 mM |
| As poly(ethylene oxide) and poly(propylene oxide) block | 0.05 g/L to 10 g/L |
| copolymer: poloxamer 188 | |
| and/or | |
| As protein agent: human serum albumin or recombinant human | 0.05 g/L to 10 g/L |
| albumin | |

TABLE 3m
Typical concentration ranges of the components of a formulation,
preferably a dry formulation such as a lyophilized formulation

| Component | Concentration Range |
|---|---|
| An enveloped virus, preferably a vesiculovirus or a vesicular | 1 × 10⁵ TCID₅₀/mL to |
| stomatitis virus, more preferably VSV-GP | 1 × 10¹² TCID₅₀/mL |
| Mannitol and/or sorbitol, preferably a combination of mannitol | total concentration of |
| and sorbitol, | 30 mM to 250 mM |
| | OR |
| | total concentration of at |
| | least 30 mM |
| A Buffer selected from the group consisting of: | 1 to 100 mM |
| acetate, citrate, histidine, succinate, HEPES, tartrate, | |
| phosphate, citrate/phosphate, lactate, and Tris; preferably Tris | |
| Arginine and/or glutamic acid | 5 to 250 mM |
| Trehalose or sucrose | 50 to 300 mM |
| As poly(ethylene oxide) and poly(propylene oxide) block | 0.5 g/L to 10 g/L |
| copolymer: poloxamer 188 | |
| and/or | |
| As protein agent: human serum albumin or recombinant human | 0.5 g/L to 10 g/L |
| albumin | |

In its broadest form the invention relates to a pharmaceutical composition comprising an enveloped virus, one or more sugar alcohols, AND a protein agent and/or a poly(ethylene oxide)/poly(propylene oxide) block copolymer. The phrase at least one of a buffer, a sugar, or an amino acid means that said formulation must contain at least one of those components but can also contain two or all three of those components. For example, a formulation comprising at least one of a buffer, a sugar, or an amino acid may contain a buffer; a sugar; an amino acid; a buffer and a sugar; a buffer and an amino acid; a buffer and a sugar and an amino acid; or, a sugar and an amino acid. It will be understood, that said formulations may comprise also e.g. two or more amino acids, two or more sugars, etc. Preferably, said formulations are provided as dry formulation such as a lyophilized formulation.

In any of the above mentioned formulations, preferably a dry formulation such as a lyophilized formulation, the poloxamer concentration can be in any concentration range between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L. Preferably the poloxamer is a poloxamer 188 and the concentration is in a range between 0.1-10 g/L.

In any of the above mentioned formulations, preferably dry formulation such as a lyophilized formulation, the protein agent concentration can be in any concentration range between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L. Preferably, the protein agent is recombinant human albumin or human serum albumin and the concentration is in a range between 0.5-10 g/L.

In any of the above-mentioned formulations, preferably dry formulation such as a lyophilized formulation, the buffer concentration can be in any concentration range between 1-100 mM, 1-90 mM, 1-80 mM, 1-70 mM, 1-60 mM, 1-50 mM, 1-40 mM, 1-30 mM, 1-20 mM, or 1-10 mM. Preferably, the formulation, preferably liquid or frozen liquid formulation comprises a Tris buffer and more preferably a Tris buffer in a concentration between 5 and 50 mM.

In any of the above-mentioned formulations, preferably dry formulation such as a lyophilized formulation, the total concentration of the one or more sugar alcohol can be in a total concentration range between 1-1000 mM, 1-900 mM, 1-800 mM, 1-700 mM, 1-600 mM, 1-550 mM, 1-500 mM, 1-450 mM, 1-400 mM, 1-350 mM, 1-300 mM, 1-250 mM, 1-200 mM, 1-150 mM, 1-100 mM, 1-50 mM, 1-20 mM, 10-1000 mM, 10-900 mM, 10-800 mM, 10-700 mM, 10-600 mM, 10-550 mM, 10-500 mM, 10-450 mM, 10-400 mM, 10-350 mM, 10-300 mM, 10-250 mM, 10-200 mM, 10-150 mM, 10-100 mM, 10-50 mM, 20-1000 mM, 20-900 mM, 20-800 mM, 20-700 mM, 20-600 mM, 20-550 mM, 20-500 mM, 20-450 mM, 20-400 mM, 20-350 mM, 20-300 mM, 20-250 mM, 20-200 mM, 20-150 mM, 20-100 mM, or 20-50 mM. In another embodiment, the total concentration of the one or more sugar alcohol will be at least 10 mM, 20 mM, 30 mM, 40 mM, or at least 50 mM. The total concentration or having a total concentration refers to the total amount of one or more sugar alcohols in the formulation, e.g. if 200 mM mannitol is present in the formulation, then the total concentration will be 200 mM. However, if e.g. 200 mM mannitol and 300 mM sorbitol are present in the formulation, then the total concentration of sugar alcohols will be 500 mM. Preferably, the formulation, preferably a dry formulation such as a lyophilized formulation, comprises mannitol and/or sorbitol and more preferably a combination of mannitol and sorbitol in a total concentration of at least 10 mM, 20 mM, 30 mM, 40 mM, or at least 50 mM.

In any of the above mentioned formulations, preferably dry formulation such as a lyophilized formulation, the sugar concentration can be in any concentration range between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM. Preferably, the sugar concentration is between 50 mM and 300 mM, between 50 mM and 250 mM, between 50 mM or 200 mM. Preferably, the dry formulation such as a lyophilized formulation, comprises a sugar, such as dextrose, fructose, galactose, glucose, raffinose, trehalose, or sucrose. More preferably the sugar is trehalose and in a concentration between 50 and 250 mM.

The pH of the above-mentioned formulations, preferably dry formulation such as a lyophilized formulation, are usually kept in a range between 5 to 9, or between 6 to 9, or between 6 to 8, or between 6.5 to 8.5, or between 6.5 to 8.0, preferably between 7.0 and 8.0.

In any of the above-mentioned formulations, preferably dry formulation such as a lyophilized formulation, the enveloped virus is preferably a vesiculovirus, more preferably a vesicular stomatitis virus and most preferred a vesicular stomatitis virus having the glycoprotein G replaced with the glycoprotein GP of the lymphocytic choriomeningitis virus (LCMV). In any of the above-mentioned formulations, preferably a dry formulation such as a lyophilized formulation, the virus concentration can be in any concentration range between 1×10⁵ TCID₅₀/mL to 1×10¹² TCID₅₀/mL or in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, or at least 1×10⁹ TCID₅₀/mL. Other ranges include between 1×10⁶ TCID₅₀/mL to 1×10¹² TCID₅₀/mL, between 1×10⁶ TCID₅₀/mL to 1×10¹¹ TCID₅₀/mL and the like.

In a preferred embodiment, a dry formulation such as a lyophilized formulation, comprises an enveloped virus, about 10 mM Tris, about, 20 mM glutamic acid, about 50 mM mannitol, about 50 mM sorbitol, about 200 mM trehalose, about 1.275 mg/ml poloxamer 188, about 5 mg/ml rHA, a pH between 7.0 and 8.0, preferably about pH 7.4. Preferably, the pH of the composition is adjusted with phosphoric acid or sodium phosphate.

In a preferred embodiment, a dry formulation such as a lyophilized formulation, comprises an enveloped virus, about 8-12 mM Tris, about, 15-25 mM glutamic acid, about 40-60 mM mannitol, about 40-60 mM sorbitol, about 180-220 mM trehalose, about 1-2 mg/mL poloxamer 188, about 4-6 mg/ml rHA, a pH between 7.0 and 8.0, preferably about pH 7.4. Preferably, the pH of the composition is adjusted with phosphoric acid or sodium phosphate.

In a preferred embodiment, a dry formulation such as a lyophilized formulation, comprises an enveloped virus, about 10 mM Tris, about, about 20 mM glutamic acid, about 100 mM arginine, about 100 mM mannitol, about 40 mM sorbitol, about 125 mM trehalose, about 2.5 mg/mL poloxamer 188, about 5 mg/ml rHA, a pH between 7.0 and 8.0, preferably about pH 7.1. Preferably, the pH of the composition is adjusted with phosphoric acid or sodium phosphate.

In a preferred embodiment, a dry formulation such as a lyophilized formulation, comprises an enveloped virus, about 8-12 mM Tris, about, about 15-25 mM glutamic acid, about 80-120 mM arginine, about 80-120 mM mannitol, about 30-50 mM sorbitol, about 100-150 mM trehalose, about 1.5-3.5 mg/mL poloxamer 188, about 4-6 mg/ml rHA, a pH between 7.0 and 8.0, preferably about pH 7.1. Preferably, the pH of the composition is adjusted with phosphoric acid or sodium phosphate.

In a preferred embodiment, a dry formulation such as a lyophilized formulation, comprises an enveloped virus, about 10 mM Tris, about, about 20 mM glutamic acid, about 25 mM citrate, about 50 mM mannitol, about 50 mM sorbitol, about 200 mM trehalose, about 2.5 mg/mL poloxamer 188, about 5 mg/ml rHA, a pH between 7.0 and 8.0, preferably about pH 7.4. Preferably, the pH of the composition is adjusted with phosphoric acid or sodium phosphate.

In a preferred embodiment, a dry formulation such as a lyophilized formulation, comprises an enveloped virus, about 8-12 mM Tris, about, about 15-25 mM glutamic acid, about 20-30 mM citrate, about 40-60 mM mannitol, about 40-60 mM sorbitol, about 180-220 mM trehalose, about 2-3 mg/mL poloxamer 188, about 4-6 mg/ml rHA, a pH between 7.0 and 8.0, preferably about pH 7.4. Preferably, the pH of the composition is adjusted with phosphoric acid or sodium phosphate.

In a preferred embodiment, a dry formulation such as a lyophilized formulation, comprises an enveloped virus, about 10 mM Tris, about, about 20 mM glutamic acid, about 150 mM arginine, about 50 mM mannitol, about 50 mM sorbitol, about 200 mM trehalose, about 1.275 mg/mL poloxamer 188, about 5 mg/ml rHA, a pH between 7.0 and 8.0, preferably about pH 7.4. Preferably, the pH of the composition is adjusted with lactic acid.

In a preferred embodiment, a dry formulation such as a lyophilized formulation, comprises an enveloped virus, about 8-12 mM Tris, about, about 15-25 mM glutamic acid, about 120-180 mM arginine, about 40-60 mM mannitol, about 40-60 mM sorbitol, about 180-220 mM trehalose, about 1-2 mg/mL poloxamer 188, about 4-6 mg/ml rHA, a pH between 7.0 and 8.0, preferably about pH 7.4. Preferably, the pH of the composition is adjusted with lactic acid.

In a preferred embodiment, a dry formulation such as a lyophilized formulation, comprises an enveloped virus, about 10 mM Tris, about, about 13 mM glutamic acid, about 100 mM arginine, about 33 mM mannitol, about 33 mM sorbitol, about 133 mM trehalose, about 1.275 mg/mL poloxamer 188, about 5 mg/ml rHA, a pH between 7.0 and 8.0, preferably about pH 7.4. Preferably, the pH of the composition is adjusted with lactic acid.

In a preferred embodiment, a dry formulation such as a lyophilized formulation, comprises an enveloped virus, about 8-12 mM Tris, about, about 10-15 mM glutamic acid, about 80-120 mM arginine, about 25-40 mM mannitol, about 25-40 mM sorbitol, about 100-150 mM trehalose, about 1-1.5 mg/mL poloxamer 188, about 4-6 mg/ml rHA, a pH between 7.0 and 8.0, preferably about pH 7.4. Preferably, the pH of the composition is adjusted with lactic acid.

In a preferred embodiment, a dry formulation such as a lyophilized formulation, comprises an enveloped virus, about 1-50 mM Tris, about 10-30 mM glutamic acid, about 100-200 mM trehalose, about 20-100 mM mannitol, about 20-100 mM sorbitol, about 0.5-5 mg/ml poloxamer 188, about 2-10 mg/ml rHA, and a pH between about 7 to 8.

In a preferred embodiment, a dry formulation such as a lyophilized formulation, comprises an enveloped virus, about 1-50 mM Tris, about 20-50 mM citrate, about 10-30 mM glutamic acid, about 100-200 mM trehalose, about 20-100 mM mannitol, about 20-100 mM sorbitol, about 0.5-5 mg/ml poloxamer 188, about 2-10 mg/ml rHA, and a pH between about 7 to 8.

In a preferred embodiment, a dry formulation such as a lyophilized formulation, comprises an enveloped virus, about 1-50 mM Tris, about 10-30 mM glutamic acid, about 50-200 mM arginine, about 100-200 mM trehalose, about 20-100 mM mannitol, about 20-100 mM sorbitol, about 0.5-5 mg/ml poloxamer 188, about 2-10 mg/ml rHA, and a pH between about 7 to 8.

In a preferred embodiment, a dry formulation such as a lyophilized formulation, comprises an enveloped virus, about 20-50 mM citrate, about 10-30 mM glutamic acid, about 100-200 mM trehalose, about 20-100 mM mannitol, about 20-100 mM sorbitol, about 0.5-5 mg/ml poloxamer 188, about 2-10 mg/ml rHA, and a pH between about 7 to 8.

In a preferred embodiment, a dry formulation such as a lyophilized formulation, comprises an enveloped virus, about 10-30 mM glutamic acid, about 50-200 mM arginine, about 100-200 mM trehalose, about 20-100 mM mannitol, about 20-100 mM sorbitol, about 0.5-5 mg/ml poloxamer 188, about 2-10 mg/ml rHA, and a pH between about 7 to 8.

In a preferred embodiment, a dry formulation such as a lyophilized formulation, comprises an enveloped virus, about 10-30 mM glutamic acid, about 100-200 mM trehalose, about 20-100 mM mannitol, about 20-100 mM sorbitol, about 0.5-5 mg/ml poloxamer 188, about 2-10 mg/ml rHA, and a pH between about 7 to 8.

TABLE 4a
Exemplary formulations

| | | | | Poly(ethylene | | |
|---|---|---|---|---|---|---|
| | | | | oxide) and | | |
| | | | | poly(propylene | | |
| | | | | oxide) block | | |
| | | Sugar/ | | copolymer | | |
| | | Sugar | | and/or | | pH |
| Formula | Buffer | alcohol | Amino acids | Protein agent | Others | (adjustment) |
| I | 20 mM | 150 mM | 20 mM | 5 g/L | — | 7.4 |
| | TRIS | Sucrose | Glutamic acid | rHA | | HCl/NaOH |
| | | 50 mM | | | | |
| | | Mannitol | | | | |
| | | 50 mM | | | | |
| | | Sorbitol | | | | |
| II | 20 mM | 100 mM | 20 mM | — | — | 7.4 |
| | TRIS | Sucrose | Glutamic acid | | | HCl/NaOH |
| | | 50 mM | | | | |
| | | Mannitol | | | | |
| | | 50 mM | | | | |
| | | Sorbitol | | | | |
| III | 20 mM | 100 mM | 120 mM | — | 0.5 mM | 7.4 |
| | TRIS | Sucrose | Alanine | | EDTA | HCl/NaOH |
| IV | 20 mM | 100 mM | 100 mM | — | 0.02 g/L | 7.4 |
| | TRIS | Sucrose | Glycine | | PS20 | HCl/NaOH |
| | | | 10 mM | | | |
| | | | Methionine | | | |
| V | 20 mM | 100 mM | 100 mM | — | 50 mM | 7.4 |
| | TRIS | Sucrose | Phenylalanine | | NaCl | Phosphoric |
| | | 7.5 mM | | | | acid/salts |
| | | Dextran | | | | |
| VI | 20 mM | 200 mM | 80 mM | — | — | 7.4 |
| | TRIS | Trehalose | Alanine | | | HCl/NaOH |
| | | 50 mM | | | | |
| | | Mannitol | | | | |
| | | 50 mM | | | | |
| | | Sorbitol | | | | |
| VII | 20 mM | 200 mM | 80 mM | — | — | 7.4 |
| | TRIS | Trehalose | Arginine | | | Phosphoric |
| | | | | | | acid/salts |
| VIII | 20 mM | 200 mM | 80 mM | — | 0.02 g/L | 7.4 |
| | TRIS | Trehalose | Arginine | | PS20 | Phosphoric |
| | | | | | | acid/salts |
| IX | 20 mM | 200 mM | 120 mM | — | — | 7.4 |
| | TRIS | Trehalose | Phenylalanine | | | HCl/NaOH |
| | | 5 mM | | | | |
| | | Sorbitol | | | | |
| X | 20 mM | 150 mM | 20 mM | 0.02 g/L | 50 mM | 7.4 |
| | TRIS | Sucrose | Glutamic acid | Poloxamer 188 | NaCl | HCl/NaOH |
| XI | 20 mM | 150 mM | 120 mM | — | — | 7.4 |
| | TRIS | Sucrose | Alanine | | | HCl/NaOH |
| XII | 20 mM | 125 mM | 100 mM | — | — | 7.4 |
| | TRIS | Sucrose | Phenylalanine | | | Phosphoric |
| | | 125 mM | | | | acid/salts |
| | | Mannitol | | | | |
| XIII | 20 mM | 150 mM | 100 mM | — | 7.5 g/L | 7.4 |
| | TRIS | Sucrose | Phenylalanine | | Dextran | Phosphoric |
| | | | | | 70 | acid/salts |

TABLE 4b
Exemplary formulations

| | | | | Poly(ethylene | | |
|---|---|---|---|---|---|---|
| | | | | oxide) and | | |
| | | | | poly(propylene | | |
| | | | | oxide) block | | |
| | | Sugar/ | | copolymer | | |
| | | Sugar | Amino | and/or | | pH |
| Formula | Buffer | alcohol | acids | Protein agent | Others | (adjustment) |
| XIV | 10 mM | 200 mM | 20 mM | 5 g/L | — | 7.4 |
| | TRIS | Sucrose | Glutamic | rHA | | Phosphoric |
| | | 50 mM | acid | | | acid/ |
| | | Mannitol | | | | Na₃PO₄ |
| | | 50 mM | | | | |
| | | Sorbitol | | | | |
| XV | 10 mM | 200 mM | 20 mM | 10 g/L | — | 7.4 |
| | TRIS | Trehalose | Glutamic | rHA | | Phosphoric |
| | | 50 mM | acid | 5 g/L | | acid/ |
| | | Mannitol | | Poloxamer 188 | | Na₃PO₄ |
| | | 50 mM | | | | |
| | | Sorbitol | | | | |
| XVI | 10 mM | 200 mM | | 5 g/L | — | 7.4 |
| | TRIS | Trehalose | | rHA | | Phosphoric |
| | | | | | | acid/ |
| | | | | | | Na₃PO₄ |
| XVII | 10 mM | 200 mM | 150 mM | 5 g/L | — | 7.4 |
| | TRIS | Trehalose | Arginine | rHA | | Phosphoric |
| | | | | 5 g/L | | acid/ |
| | | | | Poloxamer 188 | | Na₃PO₄ |
| XVIII | 10 mM | 200 mM | 150 mM | 5 g/L | — | 7.4 |
| | TRIS | Trehalose | Arginine | Poloxamer 188 | | Phosphoric |
| | | | | | | acid/ |
| | | | | | | Na₃PO₄ |
| XIX | 10 mM | 200 mM | 150 mM | 5 g/L | 10 g/L | 7.4 |
| | TRIS | Trehalose | Arginine | rHA | Dextran | Phosphoric |
| | | | | 5 g/L | 70 | acid/ |
| | | | | Poloxamer 188 | | Na₃PO₄ |
| XX | — | 200 mM | 150 mM | 5 g/L | — | 7.4 |
| | | Trehalose | Arginine | rHA | | Citric acid |
| | | | | 5 g/L | | |
| | | | | Poloxamer 188 | | |
| XXI | 10 mM | 200 mM | 150 mM | 5 g/L | — | 7.4 |
| | Histidine | Trehalose | Arginine | rHA | | Phosphoric |
| | | | | 5 g/L | | acid/ |
| | | | | Poloxamer 188 | | Na₃PO₄ |
| XXII | 10 mM | 200 mM | 50 mM | 5 g/L | — | 7.4 |
| | TRIS | Trehalose | Alanine | rHA | | Phosphoric |
| | | | 50 mM | 5 g/L | | acid/ |
| | | | Lys-HCl | Poloxamer 188 | | Na₃PO₄ |
| XXIII | 10 mM | 200 mM | 150 mM | 5 g/L | — | 7.4 |
| | TRIS | Mannitol | Arginine | rHA | | Phosphoric |
| | | | | 5 g/L | | acid/ |
| | | | | Poloxamer 188 | | Na₃PO₄ |

TABLE 4c
Exemplary formulations

| | | | | Poly(ethylene | | |
|---|---|---|---|---|---|---|
| | | | | oxide) and | | |
| | | | | poly(propylene | | |
| | | | | oxide) block | | |
| | | Sugar/ | | copolymer | | |
| | | Sugar | Amino | and/or | | pH |
| Formula | Buffer | alcohol | acids | Protein agent | Others | (adjustment) |
| XXIV | 10 mM | 200 mM | 20 mM | 5 g/L | — | 7.4 |
| | TRIS | Trehalose | Glutamic | rHA | | Phosphoric |
| | | 50 mM | acid | 5 g/L | | acid/ |
| | | Mannitol | | Poloxamer 188 | | Na₃PO₄ |
| | | 50 mM | | | | |
| | | Sorbitol | | | | |
| XXV | 10 mM | 200 mM | 20 mM | 15 g/L | — | 7.4 |
| | TRIS | Trehalose | Glutamic | rHA | | Phosphoric |
| | | 50 mM | acid | 5 g/L | | acid/ |
| | | Mannitol | | Poloxamer 188 | | Na₃PO₄ |
| | | 50 mM | | | | |
| | | Sorbitol | | | | |
| XXVI | 10 mM | 200 mM | 20 mM | 10 g/L | — | 7.4 |
| | TRIS | Trehalose | Glutamic | rHA | | Phosphoric |
| | | 50 mM | acid | 2.5 g/L | | acid/ |
| | | Mannitol | | Poloxamer 188 | | Na₃PO₄ |
| | | 50 mM | | | | |
| | | Sorbitol | | | | |
| XXVII | 10 mM | 200 mM | — | 10 g/L | — | 7.4 |
| | TRIS | Trehalose | | rHA | | Phosphoric |
| | | 50 mM | | 5 g/L | | acid/ |
| | | Mannitol | | Poloxamer 188 | | Na₃PO₄ |
| | | 50 mM | | | | |
| | | Sorbitol | | | | |
| XXVIII | 10 mM | 200 mM | 20 mM | 10 g/L | — | 7.4 |
| | TRIS | Trehalose | Glutamic | rHA | | Phosphoric |
| | | 100 mM | acid | 5 g/L | | acid/ |
| | | Mannitol | | Poloxamer 188 | | Na₃PO₄ |
| XXIX | 10 mM | 200 mM | 20 mM | 10 g/L | — | 7.4 |
| | TRIS | Trehalose | Glutamic | rHA | | Phosphoric |
| | | 100 mM | acid | 5 g/L | | acid/ |
| | | Sorbitol | | Poloxamer 188 | | Na₃PO₄ |
| XXX | 10 mM | 200 mM | 150 mM | 10 g/L | — | 7.4 |
| | TRIS | Trehalose | Arginine | rHA | | Phosphoric |
| | | | 20 mM | 5 g/L | | acid/ |
| | | | Glutamic | Poloxamer 188 | | Na₃PO₄ |
| | | | acid | | | |
| XXXI | 10 mM | 200 mM | 150 mM | 10 g/L | — | 7.4 |
| | TRIS | Trehalose | Arginine | rHA | | Phosphoric |
| | | | | 5 g/L | | acid/ |
| | | | | Poloxamer 188 | | Na₃PO₄ |
| XXXII | 10 mM | 200 mM | 50 mM | 10 g/L | — | 7.4 |
| | TRIS | Trehalose | Alanine | rHA | | Phosphoric |
| | | | 50 mM | 5 g/L | | acid/ |
| | | | Lys-HCl | Poloxamer 188 | | Na₃PO₄ |

TABLE 4d
Exemplary formulations

| | | | | Poly(ethylene | | |
|---|---|---|---|---|---|---|
| | | | | oxide) and | | |
| | | | | poly(propylene | | |
| | | | | oxide) block | | |
| | | Sugar/ | | copolymer | | |
| | | Sugar | Amino | and/or | | pH |
| Formula | Buffer | alcohol | acids | Protein agent | Others | (adjustment) |
| XXXIII | 10 mM | 200 mM | 20 mM | 5 g/L | — | 7.4 |
| | TRIS | Trehalose | Glutamic | rHA | | Phosphoric |
| | | 50 mM | acid | 5 g/L | | acid/ |
| | | Mannitol | | Poloxamer 188 | | Na₃PO₄ |
| | | 50 mM | | | | |
| | | Sorbitol | | | | |
| XXXIV | 10 mM | 200 mM | 20 mM | 10 g/L | — | 7.4 |
| | TRIS | Trehalose | Glutamic | rHA | | Phosphoric |
| | | 100 mM | acid | 5 g/L | | acid/ |
| | | Sorbitol | | Poloxamer 188 | | Na₃PO₄ |
| XXXV | 10 mM | 200 mM | 30 mM | 10 g/L | — | 7.4 |
| | TRIS | Trehalose | Glutamic | rHA | | Phosphoric |
| | | 100 mM | acid | 5 g/L | | acid/ |
| | | Sorbitol | | Poloxamer 188 | | Na₃PO₄ |
| XXXVI | 10 mM | 200 mM | 20 mM | 10 g/L | — | 7.4 |
| | TRIS | Trehalose | Glutamic | rHA | | Phosphoric |
| | | 50 mM | acid | | | acid/ |
| | | Mannitol | | | | Na₃PO₄ |
| | | 50 mM | | | | |
| | | Sorbitol | | | | |
| XXXVII | 10 mM | 200 mM | 20 mM | 10 g/L | — | 7.4 |
| | TRIS | Trehalose | Glutamic | rHA | | Phosphoric |
| | | 50 mM | acid | 2.5 g/L | | acid/ |
| | | Mannitol | | Poloxamer 188 | | Na₃PO₄ |
| | | 50 mM | | | | |
| | | Sorbitol | | | | |

TABLE 4e
Exemplary formulations

| | | | | Poly(ethylene | | |
|---|---|---|---|---|---|---|
| | | | | oxide) and | | |
| | | | | poly(propylene | | |
| | | | | oxide) block | | |
| | | Sugar/ | | copolymer | | |
| | | Sugar | Amino | and/or | | pH |
| Formula | Buffer | alcohol | acids | Protein agent | Others | (adjustment) |
| XXXVIII | 10 mM | 200 mM | 20 mM | 10 g/L | — | 7.4 |
| | TRIS | Trehalose | Glutamic | rHA | | Phosphoric |
| | | 50 mM | acid | | | acid/Na₃PO₄ |
| | | Mannitol | | | | |
| | | 50 mM | | | | |
| | | Sorbitol | | | | |
| XXXIX | 10 mM | 200 mM | 20 mM | 10 g/L | — | 7.4 |
| | TRIS | Trehalose | Glutamic | rHA | | Phosphoric |
| | | 50 mM | acid | 2.5 g/L | | acid/Na₃PO₄ |
| | | Mannitol | | Poloxamer 188 | | |
| | | 50 mM | | | | |
| | | Sorbitol | | | | |
| XXXX | 10 mM | 200 mM | 20 mM | 10 g/L | — | 7.4 |
| | TRIS | Trehalose | Glutamic | rHA | | Phosphoric |
| | | 50 mM | acid | 5 g/L | | acid/Na₃PO₄ |
| | | Mannitol | | Poloxamer 188 | | |
| | | 50 mM | | | | |
| | | Sorbitol | | | | |
| XXXXI | 10 mM | 200 mM | 20 mM | 10 g/L | 10 mM | 7.4 |
| | TRIS | Trehalose | Glutamic | rHA | Dextran | Phosphoric |
| | | 50 mM | acid | 5 g/L | 70 | acid/Na₃PO₄ |
| | | Mannitol | | Poloxamer 188 | | |
| | | 50 mM | | | | |
| | | Sorbitol | | | | |
| XXXXII | 10 mM | 200 mM | 20 mM | 10 g/L | — | 7.4 |
| | TRIS | Trehalose | Glutamic | rHA | | Phosphoric |
| | | 100 mM | acid | 5 g/L | | acid/Na₃PO₄ |
| | | Sorbitol | | Poloxamer 188 | | |
| XXXXIII | 10 mM | 200 mM | 20 mM | 10 g/L | 10 mM | 7.4 |
| | TRIS | Trehalose | Glutamic | rHA | Dextran | Phosphoric |
| | | 50 mM | acid | | 70 | acid/Na₃PO₄ |
| | | Mannitol | | | | |
| | | 50 mM | | | | |
| | | Sorbitol | | | | |
| XXXXVI | 10 mM | 200 mM | 20 mM | 5 g/L | — | 7.4 |
| | TRIS | Trehalose | Glutamic | rHA | | Phosphoric |
| | | 50 mM | acid | 5 g/L | | acid/Na₃PO₄ |
| | | Mannitol | | Poloxamer 188 | | |
| | | 50 mM | | | | |
| | | Sorbitol | | | | |
| XXXXV | 10 mM | 200 mM | 20 mM | 5 g/L | — | 7.4 |
| | TRIS | Trehalose | Glutamic | rHA | | NaOH/HCl |
| | | 50 mM | acid | 5 g/L | | |
| | | Mannitol | | Poloxamer 188 | | |
| | | 50 mM | | | | |
| | | Sorbitol | | | | |
| XXXXVI | 10 mM | 200 mM | 20 mM | 5 g/L | — | 7.4 |
| | TRIS | Trehalose | Glutamic | rHA | | NaOH/HCl |
| | | 50 mM | acid | | | |
| | | Mannitol | | | | |
| | | 50 mM | | | | |
| | | Sorbitol | | | | |

### Further Aspects:

In a second aspect, the present invention relates to a pharmaceutical composition comprising an enveloped virus, (optionally) a buffer, a sugar, at least one amino acid, one or more sugar alcohols, AND a protein agent and/or a poly(ethylene oxide)/poly(propylene oxide) block copolymer.

In one embodiment relating to the second aspect, the amino acid is selected from the group consisting of alanine, arginine, phenylalanine, glutamic acid, glycine, methionine, lysine, or glutamine, preferably glutamic acid and/or arginine.

In one embodiment relating to the second aspect, the composition is substantially free of chloride, preferably substantially free of NaCl.

In one embodiment relating to the second aspect, the pH of the composition is between 5 to 9, or between 6 to 9, or between 6.5 to 8.5, or between 6.5 to 8.0, preferably between 7.0 and 8.0.

In one embodiment relating to the second aspect, the pH of the composition is adjusted with phosphoric acid, lactic acid, citric acid, succinate acid or sodium phosphate.

In one embodiment relating to the second aspect, the buffer is selected from the group consisting of acetate, citrate, histidine, succinate, HEPES, tartrate, phosphate, citrate/phosphate, lactate, and Tris, preferably Tris.

In one embodiment relating to the second aspect, the sugar is sucrose or trehalose, preferably trehalose.

In one embodiment relating to the second aspect, the one or more sugar alcohol(s) is/are selected from the group consisting of: mannitol, sorbitol, xylitol, maltitol, maltitol symp, lactitol, inositol, glycerol erythritol, isomalt, or hydrogenated starch hydroxylate.

In one embodiment relating to the second aspect, the one or more sugar alcohol(s) is/are mannitol and/or sorbitol, preferably a combination of mannitol and sorbitol.

In one embodiment relating to the second aspect, the poly(ethylene oxide) and poly(propylene oxide) block copolymer is a poloxamer, preferably a pharmaceutically acceptable poloxamer, more preferably poloxamer 188.

In one embodiment relating to the second aspect, the protein agent is albumin, gelatin, preferably human serum albumin or recombinant human albumin.

In one embodiment relating to the first aspect, the composition comprises both a protein agent and a poly(ethylene oxide)/poly(propylene oxide) block copolymer.

In one embodiment relating to the second aspect, the protein agent is recombinant human albumin and the poly(ethylene oxide)/poly(propylene oxide) block copolymer is poloxamer 188.

In one embodiment relating to the second aspect, the composition comprises an enveloped virus, (optionally) a buffer in a concentration between 1 mM to 100 mM selected from the group consisting of acetate, citrate, histidine, succinate, HEPES, tartrate, phosphate, citrate/phosphate, lactate, and Tris, preferably a Tris buffer; glutamic acid in a concentration between 10 mM to 500 mM; a sugar in a concentration between 10 mM to 1000 mM selected from the group consisting of dextrose, fructose, galactose, glucose, raffinose, trehalose, or sucrose, preferably trehalose; mannitol in a concentration between 1-100 mM and/or sorbitol in a concentration between 1-100 mM; AND poloxamer 188 in a concentration between 0.01 g/L to 50 g/L and/or recombinant human albumin in a concentration between 0.1 g/L to 50 g/L.

In a third aspect, the invention relates to a pharmaceutical composition comprising an enveloped virus, Tris buffer, glutamic acid, and/or arginine, trehalose, mannitol and/or sorbitol, AND poloxamer 188 and/or recombinant human albumin.

In one embodiment relating to the third aspect the pharmaceutical composition comprises an enveloped virus, a Tris buffer, glutamic acid, and/or arginine, trehalose, mannitol and/or sorbitol, AND poloxamer 188 and recombinant human albumin.

In one embodiment relating to the third aspect, the poloxamer 188 is in a concentration between 0.01 g/L to 50 g/L.

In one embodiment relating to the third aspect, the recombinant human albumin is in a concentration between 0.1 g/L to 50 g/L.

In a fourth aspect, the invention relates to a pharmaceutical composition comprising an enveloped virus, (optionally) a buffer in a concentration between 1 mM to 100 mM selected from the group consisting of acetate, citrate, histidine, succinate, HEPES, tartrate, phosphate, citrate/phosphate, lactate, and Tris, preferably a Tris buffer; glutamic acid in a concentration between 10 mM to 500 mM; a sugar in a concentration between 10 mM to 1000 mM selected from the group consisting of dextrose, fructose, galactose, glucose, raffinose, trehalose, or sucrose, preferably trehalose; mannitol in a concentration between 1-100 mM and/or sorbitol in a concentration between 1-100 mM, AND poloxamer 188 in a concentration between 0.01 g/L to 50 g/L and/or recombinant human albumin in a concentration between 0.1 g/L to 50 g/L.

In one embodiment relating to the second aspect, the pharmaceutical composition comprises an enveloped virus, a Tris buffer in a concentration between 1 mM to 100 mM, glutamic acid in a concentration between 10 mM to 500 mM, trehalose in a concentration between 10 mM to 1000 mM, mannitol in a concentration between 1 mM to 100 mM and/or sorbitol in a concentration between 1 mM to 100 mM, AND poloxamer 188 in a concentration between 0.01 g/L to 50 g/L and/or recombinant human albumin in a concentration between 0.1 g/L to 50 g/L.

In one embodiment relating to any of the foregoing aspects and their embodiments, the enveloped virus is a rhabdoviridae, preferably a vesiculovirus or vesicular stomatitis virus (VSV). In a related embodiment, the enveloped virus is a recombinant vesicular stomatitis virus (VSV), wherein the gene coding for the glycoprotein G of the vesicular stomatitis virus is replaced by the gene coding for the glycoprotein GP of LCMV, and/or the glycoprotein G is replaced by the glycoprotein GP of LCMV.

In one embodiment relating to any of the foregoing aspects and their embodiments, the pharmaceutical composition is a liquid or frozen liquid pharmaceutical composition.

In a fifth aspect, the invention relates to a dry pharmaceutical composition produced by a method comprising removing water from a pharmaceutical composition, said composition comprising an enveloped virus, a Tris buffer in a concentration between 1 mM to 100 mM, glutamic acid in a concentration between 10 mM to 500 mM, trehalose in a concentration between 10 mM to 1000 mM, mannitol in a concentration between 1 mM to 100 mM and/or sorbitol in a concentration between 1 mM to 100 mM, AND poloxamer 188 in a concentration between 0.01 g/L to 50 g/L and/or recombinant human albumin in a concentration between 0.1 g/L to 50 g/L. In a related embodiment the dry pharmaceutical composition is obtained by freeze-drying/lyophilization, or spray-drying. In a further related embodiment, the method comprises placing the pharmaceutical composition in a vacuum under controlled temperatures and pressure to remove the water. In a related embodiment, the method is lyophilization. In another embodiment, the dry pharmaceutical composition comprises less than about (0.1%-10%) w/w water.

In a sixth aspect, the invention relates to a pharmaceutical composition comprising water and the dry pharmaceutical composition of the third aspect and any of its embodiments.

In one embodiment relating to any of the foregoing aspects and their embodiments, the pharmaceutical composition is a dry pharmaceutical composition.

In another embodiment relating to any of the foregoing aspects and their embodiments, the pharmaceutical composition according further comprises arginine, preferably in a concentration between 10 mM to 500 mM.

In a seventh aspect, the invention relates to a lyophilized formulation that, when reconstituted in water, results in a reconstituted solution comprising an enveloped virus, a Tris buffer in a concentration between 1 mM to 100 mM, glutamic acid in a concentration between 10 mM to 500 mM, trehalose in a concentration between 10 mM to 1000 mM, mannitol in a concentration between 1 mM to 100 mM and/or sorbitol in a concentration between 1 mM to 100 mM, AND poloxamer 188 in a concentration between 0.01 g/L to 50 g/L, and/or recombinant human albumin in a concentration between 0.1 g/L to 50 g/L.

### EXAMPLES

### Methods:

### Formulations and Tested Viruses

The tested viruses were vesicular stomatitis viruses having the wild-type glycoprotein G replaced with the glycoprotein GP of the lymphocytic chorio-meningitis virus; such virus is named subsequently VSV-GP. In some instances, the VSV-GP encodes additionally for a cargo and such virus is named subsequently VSV-GP-Cargo1/2/or3. The virus material VSV-GP or VSV-GP-cargo1/2/or 3 is used undiluted at a concentration of approx. 5×10⁹ TCID₅₀/ml. To transfer the virus in specific formulations, dialysis cassettes (Slide-a-Lyzer Dialysis Cassette, Thermo, MWCO 10 kDa or 20 kDa, 12 to 30 ml) are used. Samples are dialyzed three times each for 2 h slowly stirred at a temperature of 2-8° C. or room temperature. The last step is performed overnight. Sterile filtration is done using 0.22 μm PES filters. For macromolecules that do not pass the dialysis membrane, i.e., recombinant human albumin (rHA), dextran, and poloxamer 188, appropriate volumes of stock solutions are added after dialysis. For placebo samples, the amount of rHA and poloxamer 188 is spiked to 15 ml of the respective sterile filtered formulations.

### Method to Determine Infectivity (TCID₅₀)

### Cells and Viruses:

BHK-21 cells (#603126 (C13), CLS) are cultured in 5% CO₂ at a temperature of 37° C. Medium (GMEM #21710082, Thermo) is supplemented with 8.7% FCS and 4.3% tryptose phosphate broth. BHK-21 cells are washed with PBS and detached from the cell culture flask by incubation with TrypLE™ Select Enzyme at a temperature of 37° C. for 6-8 min. Cells are taken up in medium, counted using the Flex2 (nova biomedical) and seeded on 96-well plates.

### TCID₅₀ Assay:

In 96-well plates 104 BHK-21 cells in 100 μl supplemented GMEM are seeded per well. Following an incubation for 24 h, the adherent cells are infected with 11×0.5 log 10 serial dilutions of the virus or the diluent alone (negative control) before incubation for three days at a temperature of 37° C. and 5% CO₂. Brightfield images of the cell culture wells are taken with the Cytation5 Multi-Mode Imaging Reader (BioTek) using a 4× magnifying objective. Whether the imaged wells are CPE positive or negative is assessed by eye (visually). The final titer [TCID₅₀/mL] is calculated by the formula of Spearman and Karber. For each virus sample infections with serial dilutions are performed in a total of eight plates at the same day. Based on those eight replicates the TCID₅₀/mL is calculated. As assay control, (VSV-crude-harvest material, stored at a temperature of −80° C. is used and conducted at each time point in a separate 96-well-plate.

### Method to Determine Visible and Subvisible Particles

### Detection of Visible Particles by Visual Inspection (VI)

Visual inspections are performed in accordance with internal standards in a stepwise procedure. The inspection is performed by two trained examiners.
Step 1: Prior to inspection, the samples are equilibrated to room temperature in the dark.
Step 2: The vial is placed outside the inspection zone on the workbench without being swirled or inverted. The bottom of the vial as well as the meniscus of the vial is inspected for particles.
Step 3: The vial is inspected for sediment on the bottom of the vial outside of the inspection zone, directly in front of the light source. The vial is inspected in an upright position and handled carefully to avoid swirling up a potential sediment.
Step 4: The vial is inspected outside of the inspection zone, directly in front of the light source. The vial is held upright and swirled. If there is a sediment on the bottom of the vial, it would rise and form a swirlable subvisible particle sediment.
Step 5: The vial is inspected for the presence or absence of visible particles in compliance to the European Pharmacopoeia (9th edition; monograph 2.9.20) at 2,000-3,750 lux. By avoiding the formation of air bubbles, the solution is homogenized by gentle swirling and the liquid is inspected for 5 sec in front of a white and 5 sec in front of a black background in the inspection area.
Step 6: As a last step, the vial is inspected outside of the inspection zone, directly in front of the light source. The vial is carefully swirled. The sample is inspected for very small particles, which are only visible if present in high numbers.

### Detection of Sub-Visible Particles by MFI

MFI measurements are conducted using a MFI-5200 particle analyzer system equipped with a silane coated high resolution 100-μm flow cell. In brief, samples are diluted 5-fold in ADB. A pre-run volume of 0.25 ml is followed by a sample run of 0.6 ml. Between the measurements, the flow cell is rinsed with water. The background illumination is optimized by using water. MFI View System Software (MVSS) version 2-R5.0.0.43 is used to perform the measurements and MFI View Analysis Suite (MVAS) software version 1.3.0.1007 is used to analyze the samples.

### Lyophilization

Samples are lyophilized using a LCD-2-6D or LCD-2-10D pilot-scale freeze dryer (Martin Christ Gefriertrocknungsanlagen GmbH, Osterode, Germany) in 2R vials (0.4 ml) with Flurotec® stoppers. The vacuum during the freeze-drying process is controlled by a capacitance manometer. Based on the calculated solid contents as well as the estimated glass transition temperature of the maximally freeze-concentrated solution (T_{g}′) a conservative lyophilization process is applied. During the freeze-drying process, product temperature, shelf temperature, condenser temperature, and chamber pressure (capacitance manometer and Pirani gauge) are monitored. The product temperature is monitored by eight Pt₁₀₀ sensors (1 sensor per formulation), which are placed in different vials located in the middle of the samples of the third shelf. At the end of the process, the stoppers of the vials are closed in the freeze-dryer under nitrogen atmosphere at a pressure of 600 mbar. After stoppering the vials, the chamber is aerated to atmospheric pressure by using nitrogen, and the samples are removed. The samples are crimp-capped after removal from the freeze-dryer, labeled, and stored at the respective storage conditions for further analysis. Lyo formulations are stored at a temperature of 25° C. or 30° C. in ICH110 Cabinets (Memmert GmbH & Co. KG, Schwabach, Germany).

### Determining the Glass Transition Temperature

The glass transition temperatures T_{g} and T_{g}′ are determined on a DSC 214 Polyma oven (Erich Netzsch GmbH & Co Holding KG, Selb, Germany). Two to ten mg lyophilized product ware weighed in aluminum pans in a glove box with controlled humidity (approx. 8% rH) and subsequently sealed in aluminum pans. T_{g} values are analyzed with Netzsch Proteus Analysis Software. All measurements are performed as duplicates and the results are calculated as the mean±standard deviation.

### Frozen Liquid Sample Preparation

Samples are frozen by placing samples in a freezer at a temperature of −70° C. using a CoolCell LX BioCision LLC (Larkspure, CA). The temperature is recorded by monitored by thermo-loggers. For freeze/thaw cycling, samples are removed from the freezer and placed into ICH110 Cabinets (Memmert GmbH & Co. KG, Schwabach, Germany) for at least 2 h at a temperature of 25° C.±2° C./60%±5% rH or at a temperature of 30° C.±2° C./65%±5% rH, respectively. Temperature cycling is repeated 1-, 3-, or 5-times.

### Determination of Particle Count by Nanoparticle-Tracking Analysis (NTA)

Particle count is determined by nanoparticle tracking analysis (NTA) with NanoSight NS300 (Malvern, Worcestershire, UK). Samples are diluted in appropriate particle free dilution buffer to have approx. 30 to 120 particles per frame. Particles are tracked 5 times for 60 seconds in dynamic mode. For each sample two independent dilutions are prepared. Particle counts are analyzed using NanoSight NTA 3.4 Software (Malvern, Worcestershire, UK) in raw data mode with a detection threshold of approx. 6.

### Example 1

### Initial Screening Formulations

Outline: The objectives of the initial formulation development activities were to characterize different virus formulations (Tables 5 a-c), by examining the influence of storage and freeze/thaw (F/T) cycles on infectivity (TCID₅₀) and the formation of particles in formulations designed to be stored as a liquid, a frozen liquid, or as a dry formulation, i.e., being lyophilized. In this example VSV-GP-Cargo1 was tested. In the following, WP3a formulations refer to the liquid formulations, WP3b formulations refer to the frozen liquid formulations and WP3d formulations refer to the lyophilized formulations. The general formulation was the same for the initial liquid, frozen liquid, or lyophilized formulations.

TABLE 5a
Initial liquid (WP3a) formulations

| | | General | | | | |
|---|---|---|---|---|---|---|
| | Excipient | Formulation | Liquid 1 | Liquid 2 | Liquid 3 | Liquid 4 |
| Buffer | TRIS [mM] | 20 | 20 | 20 | 20 | 20 |
| | pH 7.4 adjustment | NaOH | NaOH | NaOH | NaOH | Na₃PO₄ |
| Sugar and | Sucrose [mM] | 150 | 100 | 100 | 100 | 100 |
| polysaccharides | Trehalose [mM] | | | | | |
| | Dextran 70 [g/l] | | | | | 7.5 |
| Sugar alcohol | Mannitol [mM] | 50 | 50 | | | |
| | Sorbitol [mM] | 50 | 50 | | | |
| Salt | NaCl [mM] | | | | | 50 |
| Amino acids | Ala [mM] | | | 120 | | |
| | Arg [mM] | | | | | |
| | Phe [mM] | | | | | 100 |
| | Glu [mM] | 20 | 20 | | | |
| | Gly [mM] | | | | 100 | |
| | Met [mM] | | | | 10 | |
| Detergent | Poloxamer 188 [g/l] | | | | | |
| | Polysorbate 20 [g/l] | | | | 0.02 | |
| Macromolecule | rHA [g/l] | 5 | | | | |
| Others | EDTA [mM] | | | 0.5 | | |

TABLE 5b
Initial frozen liquid (WP3b) formulations

| | | General | | | | |
|---|---|---|---|---|---|---|
| | Excipient | Formulation | FroLiq 1 | FroLiq 2 | FroLiq3 | FroLiq 4 |
| Buffer | TRIS [mM] | 20 | 20 | 20 | 20 | 20 |
| | pH 7.4 adjustment | NaOH | NaOH | NaOH | Na3PO4 | Na3PO4 |
| Sugar and | Sucrose [mM] | 150 | | | | |
| polysaccharides | Trehalose [mM] | | 150 | 150 | 125 | 150 |
| | Dextran 70 [g/l] | | | | | 7.5 |
| Sugar alcohol | Mannitol [mM] | 50 | | | 125 | |
| | Sorbitol [mM] | 50 | | | | |
| Salt | NaCl [mM] | | 50 | | | |
| Amino acids | Ala [mM] | | | 120 | | |
| | Arg [mM] | | | | | |
| | Phe [mM] | | | | 100 | 100 |
| | Glu [mM] | 20 | 20 | | | |
| | Gly [mM] | | | | | |
| | Met [mM] | | | | | |
| Detergent | Poloxamer 188 [g/l] | | 0.02 | | | |
| | Polysorbate 20 [g/l] | | | | | |
| Macromolecule | rHA [g/l] | 5 | | | | |

TABLE 5c
Initial lyo (WP3d) formulations

| | | General | | | | |
|---|---|---|---|---|---|---|
| | Excipient | Formulation | Lyo 1 | Lyo 2 | Lyo 3 | Lyo 4 |
| Buffer | TRIS [mM] | 20 | 20 | 20 | 20 | 20 |
| | pH 7.4 adjustment | NaOH | NaOH | Na₃PO₄ | Na₃PO₄ | NaOH |
| Sugar and | Sucrose [mM] | 150 | | | | |
| polysaccharides | Trehalose [mM] | | 200 | 200 | 200 | 200 |
| | Dextran 70 [g/l] | | | | | |
| Sugar alcohol | Mannitol [mM] | 50 | 50 | | | |
| | Sorbitol [mM] | 50 | 50 | | | 5 |
| Salt | NaCl [mM] | | | | | |
| Amino acids | Ala [mM] | | 80 | | | |
| | Arg [mM] | | | 80 | 80 | |
| | Phe [mM] | | | | | 120 |
| | Glu [mM] | 20 | | | | |
| | Gly [mM] | | | | | |
| | Met [mM] | | | | | |
| Detergent | Poloxamer 188 [g/l] | | | | | |
| | Polysorbate 20 [g/l] | | | | 0.02 | |
| Macromolecule | rHA [g/l] | 5 | | | | |

### Effects of Storing Formulations at a Temperature of 5° C. or 25° C. on the Infectious Titer and SvPs in Liquid Formulations (WP3a)

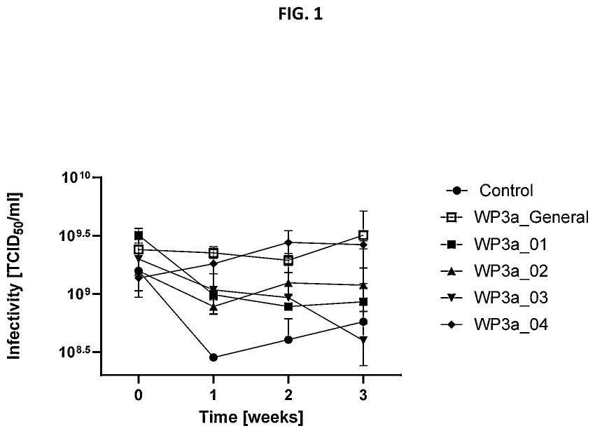

### FIGS. 1 to 4

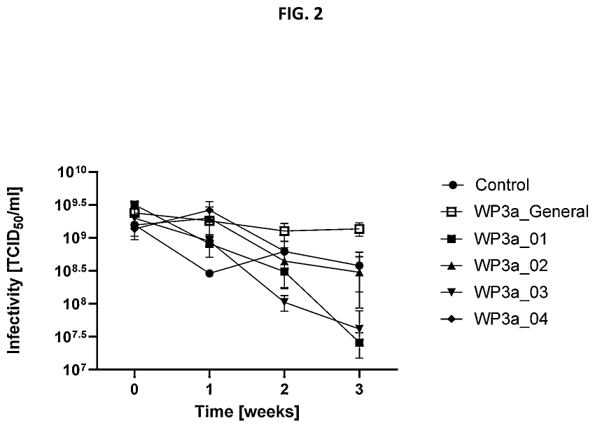

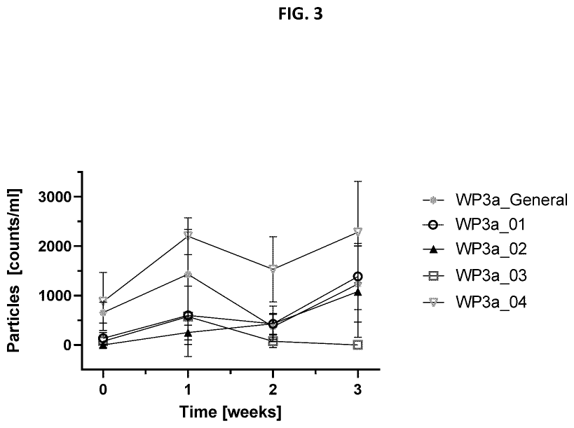

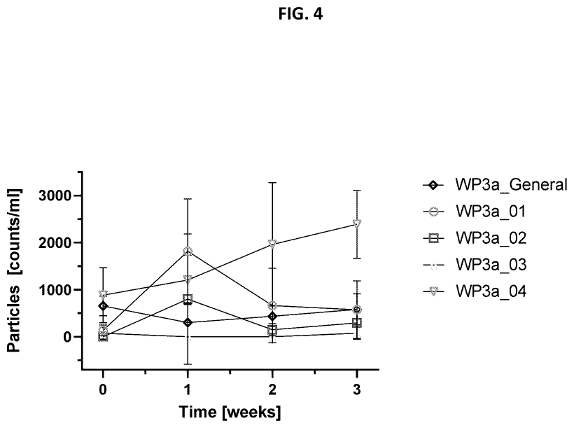

All liquid formulations (except WP3a_03 containing polysorbate at 0.02 g/L) indicated a decent stability during storage for 28 days at a temperature of 5° C. (FIG. 1) with an average loss of titer during storage of 0.2 log units and also for some formulations at a temperature of 25° C. (FIG. 2) with a loss of titer in a between 0.2-2.1 log units. The most stable formulation was WP3a_General containing rHA (at 5 g/L). All formulations had very low levels of sub visible particles (SvPs). The formulation with the lowest amount of SvPs≥10 μm after storage at a temperature of 5° C. (FIG. 3) or at 25° C. (FIG. 4) was WP3a_03 containing polysorbate.

### Effects of F/T Cycles on the Infectious Titer and Subvisible Particle Formation in Liquid Formulations (WP3a)

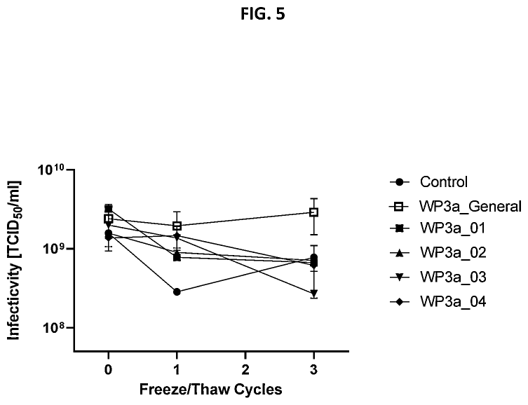

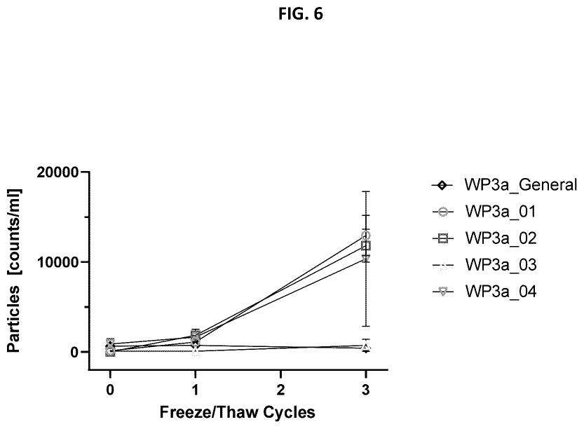

### FIGS. 5 to 6

When liquid formulations of WP3a were stressed with one or three F/T cycles from a temperature of −80° C. to +25° C. (Table 6) three F/T cycles led to higher loss of the infectious titer than one F/T cycle. The most stable formulation was WP3a_General containing rHA. The formulation containing polysorbate (WP3a_03) exhibited the highest loss in titer (FIG. 5) and was thus, no longer considered as an excipient for formulation development despite of the positive impact of polysorbate on particle formation. The negative effect of polysorbate on the stability of VSV likely stems from its effect on its phospholipid envelope, that can easily be damaged by a detergent. F/T cycling increased the amount of SvPs in all formulations except for WP3a_General containing rHA with respect to particles ≥10 UM and for WP3a_03 containing polysorbate with respect to particles ≥10 UM (FIG. 6).

TABLE 6
Stress and storage conditions of virus in liquid formulations
and without or with additional freeze/thaw cycles (F/T)

| | | | | Storage of virus | | |
|---|---|---|---|---|---|---|
| | F/T | t = 0 | | formulations in days | | |
| | (−80° C./25° C.) | (Pre F/T) | Post F/T | t = 7 | t = 14 | t = 28 |
| 5° C. | 0 | X | | X | X | X |
| | 1 | | X* | X | | |
| | 3 | | X* | X | | |
| 25° C./60% rH | 0 | | | X | X | X |
| | 1 | | X* | X | | |
| | 3 | | X* | X | | |

*5° C. & 25° C. samples and measurements of 1 & 3 F/T are the same.

### Effects of F/T Cycles on the Infectious Titer and Subvisible Particle Formation in Frozen Liquid Formulations (WP3b)

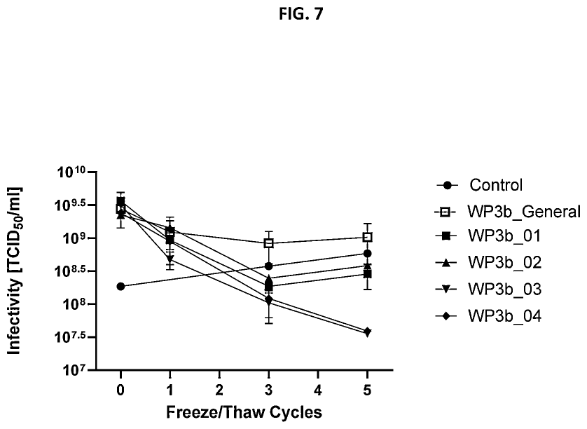

### FIGS. 7 to 10

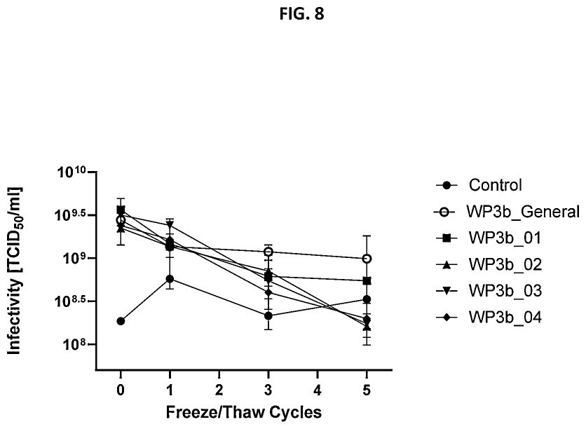

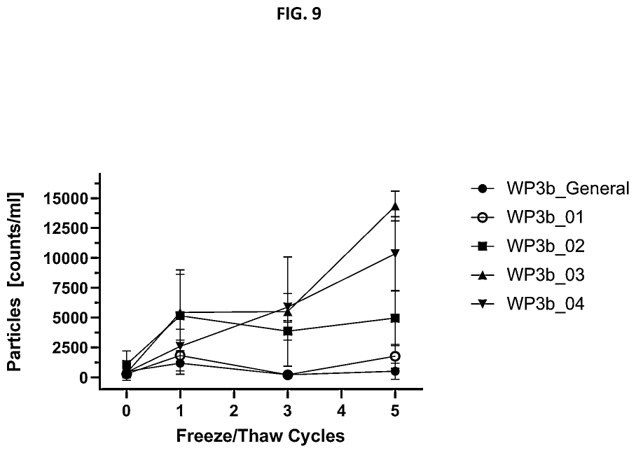

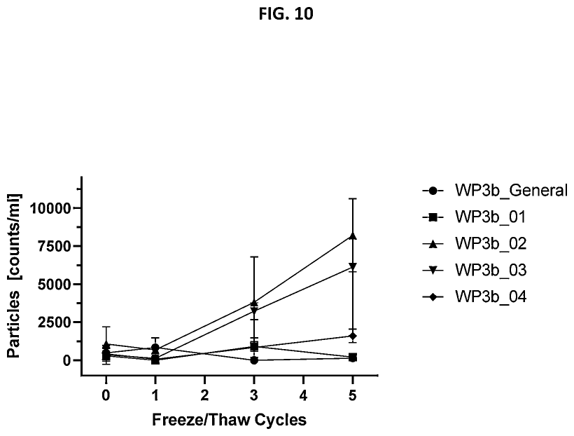

When VSV-GP was cycled from temperatures of −20° C. or −80° C. to ±25° C., one, three, or five times (Table 7), the most stable formulation was again WP3a_General, containing rHA followed by the formulation containing poloxamer 188, regardless of whether the formulation was cycled from a temperature of −20° C. to +25° C. (FIG. 7) or from a temperature of −80° C. to +25° C. (FIG. 8). Only in those formulations, five F/T cycles led to no further loss of the infectious titer in comparison to three F/T cycles. Also, in those formulations, the number of SvPs≥10 UM did not increase by F/T, neither when formulations were cycled from a temperature of −20° C. to +25° C. (FIG. 9) or from a temperature of −80° C. to +25° C. (FIG. 10). The number of particles increased by more than 2-fold when cycled at a temperature of −20° C. as compared to −80° C. In view of the earlier observation, when detergents such as polysorbate damaged the envelope of VSV-GP and caused an unacceptable drop in the infectious titer, it was very surprising that the detergent poloxamer 188 did not have such an effect on the envelope and titer. In contrast, the addition of poloxamer 188 to the formulation was beneficial to both, the infectious titer and the suppression of SvPs formation.

TABLE 7
Freeze/thaw cycles at different storage conditions
of virus in frozen-liquid formulations

| | Virus formulations detected after freeze/thaw cycles | | | |
|---|---|---|---|---|
| | 0 F/T | 1 F/T | 3 F/T | 5 F/T |
| −20° C. | X | X | X | X |
| −80° C. | | X | X | X |

### Effects of Storing Formulations at a Temperature of 25° C. on the Infectious Titer and Subvisible Particle Formation in Lyophilized Formulations (WP3d)

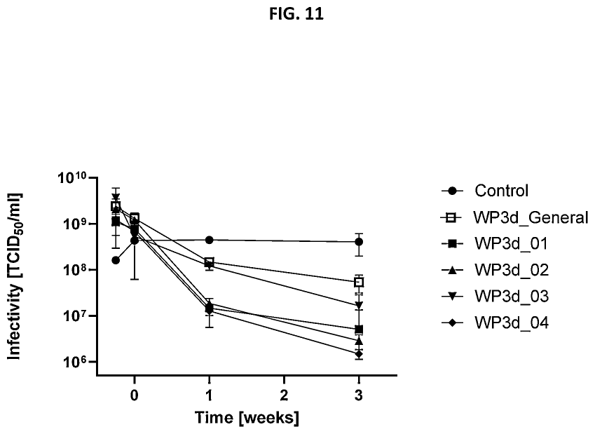

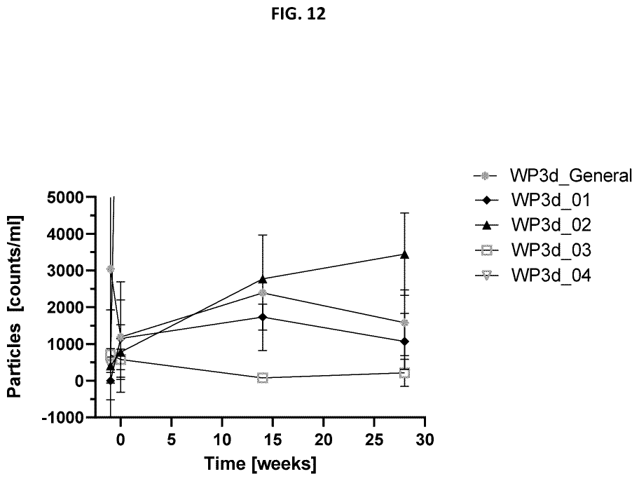

### FIGS. 11 to 12

The infectivity in all formulations was stable immediately after lyophilization but decreased almost linearly when stored at a temperature of 25° C. (FIG. 11). All formulations had very low levels of SvPs. The formulations with the lowest amount of SvPs≥10 μm (FIG. 12) were, as expected, WP3d_General, containing rHA and WP3d_03 containing polysorbate. All lyophilized formulations resulted in stable lyo cakes except WP3d_General. The reason was that in this formulation sucrose was the sugar component instead of trehalose, which was in all other lyo formulations. Since the glass transition temperature (T_{g}) value of WP3d_General was close to the storage temperature at approx. 25° C., sucrose was exchanged in the following experiments with trehalose.

Overall, WP3a_General=WP3b_General=WP3d_General, containing rHA performed well in all stress conditions and served as the basis for further lyo formulation development efforts. The composition was 20 mM Tris (titrated with NaOH), 150 mM sucrose, 50 mM mannitol, 50 mM sorbitol, 20 mM glutamic acid, 5 g/L rHA, pH 7.4.

### Example 2

### First Optimization Round for Lyo-Formulations

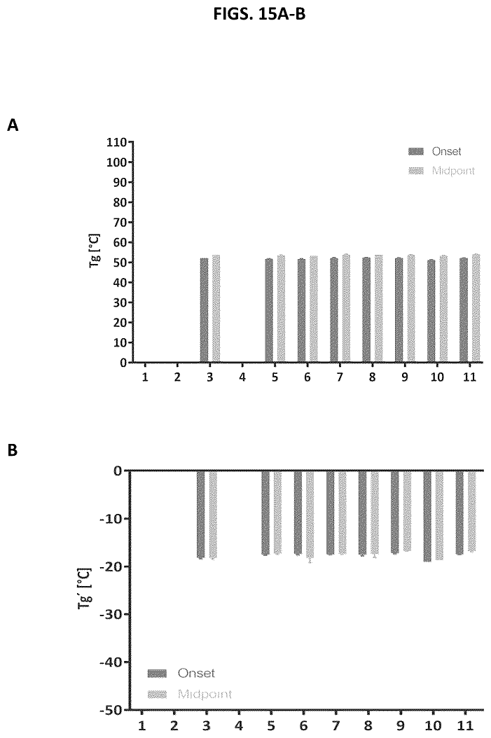

### FIGS. 15A-B

The objective of WP7 was to identify critical parameters of the formulation and the lyophilization process to stabilize oncolytic viruses after lyophilization and storage, based on previous learnings from WP3. Therefore, selected formulations were stored at a temperature of 5° C. or 25° C. for two weeks and the infectious titer was determined. To optimize the formulation, the amount of Tris buffer was reduced, sucrose was exchanged for trehalose and its concentration was increased to 200 mM. In addition, poloxamer 188 was added to the formulation. However, in this particular experiment poloxamer 188 and dextran was not spiked to the formulation after dialysis but mistakenly added to the dialysis buffer. In this set-up, only monomers of poloxamer 188 with an average MW of 8.6 kDa pass through the dialysis membrane with a MWCO of 10 kDa but not poloxamer micelles. As a result, the actual poloxamer concentration in the formulations could only be determined in placebo samples but was below the limit of quantification of the assay. The numerical poloxamer concentration was subsequently externally determined at 0.03-0.06 g/L, i.e., at a concentration approximately 100-times less than the intended concentration. Nonetheless, T_{g} and T_{g}′ curves of lyophilized and respectively reconstituted placebo formulations confirmed the presence of poloxamer 188 in the formulations by a second melting transition (T_{g}) peak and a second thermal event in all poloxamer-containing formulations (TG curves not shown but onset of and midpoint of temperatures of a second melting transition and second glass transition peak shown in FIGS. 15A and 15B). Formulations 1, 2, and 4 that did not contain poloxamer 188 were lacking these peaks. In the formulation tables, the poloxamer 188 concentration was thus reported at ˜0.05 g/L. Since the concentration of dextran was not determined, the concentration was indicated with a question mark. All formulations with incorrect amounts of excipients were tested again in a later work package in the intended correct amounts.

TABLE 8
WP7 formulations

| | Excipient | WP7_01 | WP7_02 | WP7_03 | WP7_04 | WP7_05 |
|---|---|---|---|---|---|---|
| Buffer | Histidine [mM] | | | | | |
| | TRIS [mM] | 20 | 20 | 20 | 20 | 20 |
| | pH 7.4 adjustment | NaOH | NaOH | NaOH | NaOH | Na₃PO₄ |
| Sugar and | Sucrose [mM] | 150 | 200 | | | |
| polysaccharide | Trehalose [mM] | | | 200 | 200 | 200 |
| | Dextran 70 [g/l] | | | | | |
| Sugar alcohol | Mannitol [mM] | 50 | 50 | 50 | | |
| | Sorbitol [mM] | 50 | 50 | 50 | | |
| Amino acids | Ala [mM] | | | | | |
| | Arg [mM] | | | | | 150 |
| | Glu [mM] | 20 | 20 | 20 | | |
| | Lys-HCL [mM] | | | | | |
| Detergent | Poloxamer 188 [g/L] | | | ~0.05 | | ~0.05 |
| Macromolecule | rHA [g/L] | 5 | 5 | 10 | 5 | 5 |

TABLE 8
WP7 formulations

| | Excipient | WP7_06 | WP7_07 | WP7_08 | WP7_09 | WP7_10 | WP7_11 |
|---|---|---|---|---|---|---|---|
| Buffer | Histidine [mM] | | | | 10 | | |
| | TRIS [mM] | 20 | 20 | 20 | 20 | 20 | 20 |
| | pH 7.4 adjustment | NaOH | Na₃PO₄ | Na₃PO₄ | NaOH | NaOH | NaOH |
| Sugar and | Sucrose [mM] | | | | | | |
| polysaccharides | Trehalose [mM] | 200 | 200 | 200 | 200 | 200 | |
| | Dextran 70 [g/l] | | 10 | | | | |
| Sugar alcohol | Mannitol [mM] | | | | | | 200 |
| | Sorbitol [mM] | | | | | | |
| Amino acids | Ala [mM] | | | | | 50 | |
| | Arg [mM] | 150 | 150 | 150 | 150 | | 150 |
| | Glu [mM] | | | | | | |
| | Lys-HCL [mM] | | | | | 50 | |
| Detergent | Poloxamer 188 [g/L] | ~0.05 | ~0.05 | ~0.05 | ~0.05 | ~0.05 | ~0.05 |
| Macromolecule | rHA [g/L] | | 5 | 5 | 5 | 5 | 5 |

Effects of the Sugar Species on Lyo Cake Stability and Effects of Storing Formulations at a Temperature of 5° C. or 25° C. on the Infectious Titer of Lyophilized Formulations from WP7

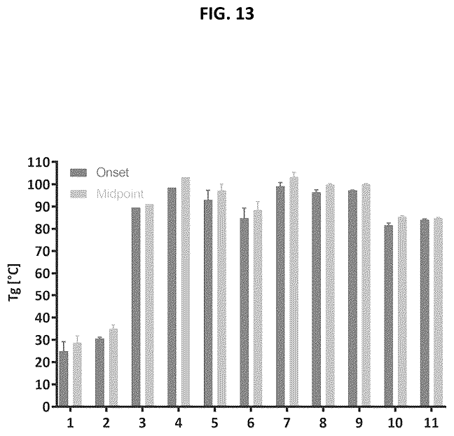

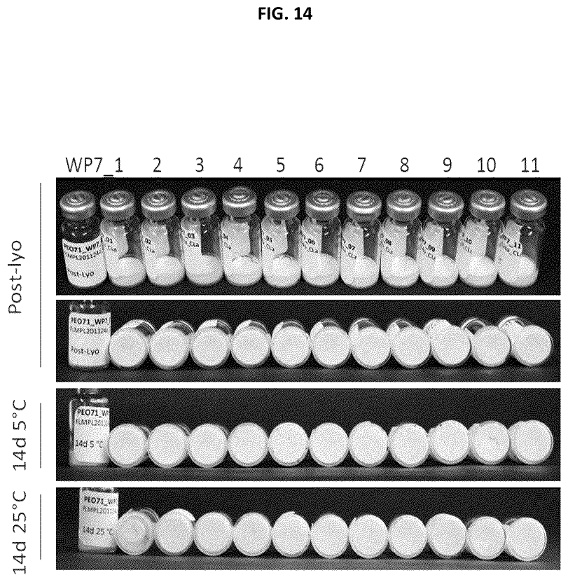

### FIGS. 13, 14 & 16

Lyo cakes containing sucrose with T_{g} values close to 25° C. melted upon storage at a temperature of 25° C. but melting could be avoided by using trehalose with a higher T_{g} value instead of sucrose in the formulation. Trehalose is particularly beneficial in lyophilized formulations since it has an anomalously high T_{g} value, appox. 60° C. higher than that of sucrose (Zhang, M., Oldenhof, H., Sydykov, B. et al. Freeze-drying of mammalian cells using trehalose: preservation of DNA integrity. Sci Rep 7, 6198 (2017). Previous results of WP3 were confirmed even with minor adaptations of the formulation. T_{g} onset values of the sucrose-containing formulation that was titrated with HCl/NaOH were at approx. 25° C. When the formulation was titrated with Na₃PO₄/H₃PO₄, the T_{g} onset values increased just above room temperature at approx. 25° C. When the formulation was titrated with Na₃PO₄/H₃PO₄, and sucrose was exchanged with trehalose, the T_{g} onset values increased to approx. 90° C. (FIG. 13). Accordingly, the lyo cakes remained stable when stored at a temperature of 25° C. (FIG. 14).

The infectious titer after lyophilisation and after 14 days at a temperature of 25° C. remained within assay variation (˜0.5 log units) for 4 lead formulations. Despite the mistakenly low amount of poloxamer 188 in the formulation (˜0.05 g/L instead of 5 g/L), its presence could be detected in T_{g} and T_{g}′ curves of lyophilized placebo formulations. The onset of the T_{g} peak was at approx. 52° C., i.e., the melting temperature of poloxamer 188 (FIG. 15A). The onset of the T_{g}′ peak was at approx. −17° C. (FIG. 15B). Both peaks were absent in formulations (1, 2, & 4), which did not contain poloxamer 188.

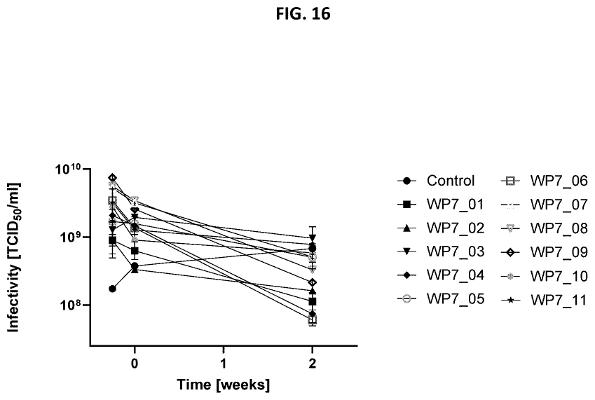

Infectivity results after storing lyophilized formulation for up to two weeks at a temperature of 25° C. (FIG. 16) indicated that formulations lacking poloxamer 188 suffered the highest loss of infectivity, whereas even small amounts of poloxamer 188 in the formulation at approx. 0.05 g/l poloxamer 188 were beneficial in stabilizing the lyophilized formulation upon storage and particularly in combination with rHA. These effects were further investigated in the next steps of this study (WP9-WP11). The excipients alanine, arginine, and lysine had no further benefit in the lyophilized formulations and were thus not included in further studies. Since titrating the pH with trisodium Na₃PO₄/H₃PO₄ instead of NaOH/HCl increased the T_{g} value slightly, the pH of all subsequent formulations was titrated with Na₃PO₄/H₃PO₄. The best performing composition in this round of experiments was 10 mM Tris (titrated with Na₃PO₄), 200 mM trehalose, 50 mM mannitol, 50 mM sorbitol, 20 mM glutamic acid, 10 g/L rHA, and ˜0.05 g/L poloxamer 188, pH 7.4.

TABLE 9
Lyophilization and storage of formulated samples

| | | t = 0 | |
|---|---|---|---|
| Stress Type/Time Point | Pre-Lyo | (Post-Lyo) | 2 weeks |
| 5° C. ± 3° C. | X | X | X |
| 25° C. ± 2° C./ | | | X |
| 60% RH ± 5% RH1) | | | |

### Example 3

### Third Optimization Round Formulations

The aim of this optimization round was to verify previous results using a representative VSV-GP-Cargo1 (WP9a) batch and a new virus variant, i.e., VSV-GP-Cargo2. VSV-GP-Cargo2 (WP9b) is intended for a different therapeutical indication and is therefore manufactured and applied at a concentration, approx. 100-times-less than VSV-GP-Cargo1. Both viruses were formulated in four selected formulations. In this round of optimization, poloxamer 188 was added at the originally intended concentration of 5 g/L and not at the mistakenly lower concentration of 0.05 g/L. VSV-GP-Cargo2 was also stored in two additional formulations containing poloxamer at 2.5 g/L or 0 g/L and all formulations were stored at a temperature of 5° C. or at 25° C. for up to 12 months. The infectious titer and SvP concentrations after reconstitution were determined in regular time intervals. Furthermore, the benefits of the mixture of sugar alcohols and optimum concentration of rHA was evaluated.

TABLE 10
WP9 formulations

| | Excipient | WP9_01 | WP9_02 | WP9_03 | WP9_04 | WP9_05 | WP9_06 |
|---|---|---|---|---|---|---|---|
| Buffer | TRIS [mM] | 10 | 10 | 10 | 10 | 10 | 10 |
| | pH 7.4 adjustment | Na₃PO₄ | Na₃PO₄ | Na₃PO₄ | Na₃PO₄ | Na₃PO₄ | Na₃PO₄ |
| Sugar | Trehalose [mM] | 200 | 200 | 200 | 200 | 200 | 200 |
| Sugar alcohol | Mannitol [mM] | 50 | 50 | | | 50 | 50 |
| | Sorbitol [mM] | 50 | 50 | 100 | 100 | 50 | 50 |
| Amino acid | Glutamic acid [mM] | 20 | 20 | 20 | 30 | 20 | 20 |
| Detergent | Poloxamer 188 [g/l] | 5 | 5 | 5 | 5 | | 2.5 |
| Macromolecule | rHA [g/l] | 10 | 5 | 10 | 10 | 10 | 10 |

### Effects of Storing Formulations at a Temperature of 5° C. or 25° C. on the Infectious Titer of Lyophilized Formulations Containing Representative Batches of VSV-GP-Cargo1 (WP9a) or VSV-GP-Cargo2 (WP9b)

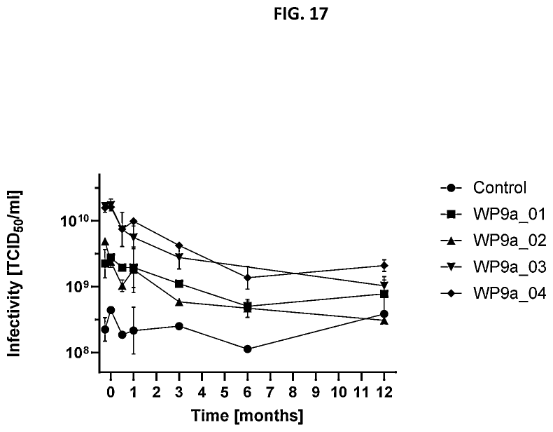

### FIGS. 17 to 20

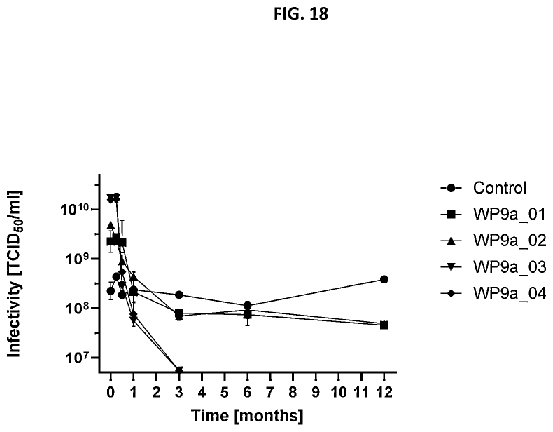

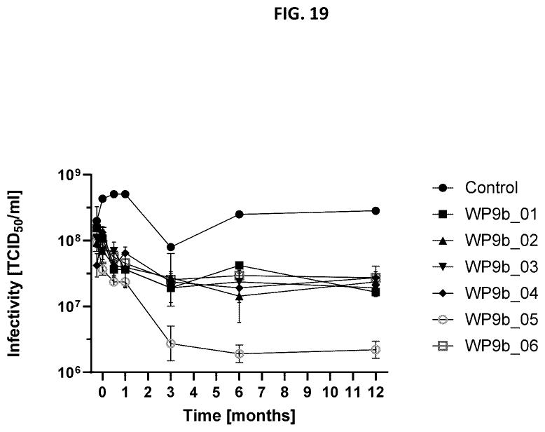

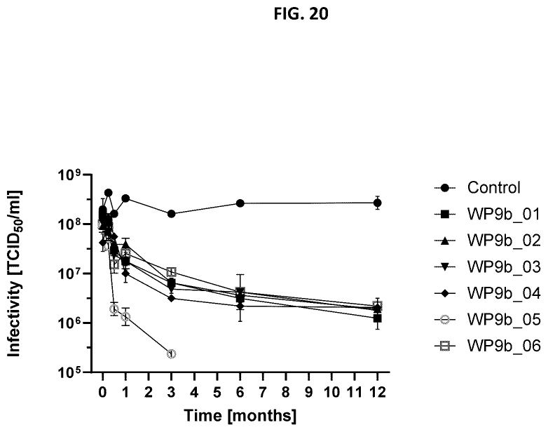

A rHA concentration of 5 g/L in VSV-GP-Cargo1 formulations (lower concentrations were not tested) was sufficient to maintain the infectious titer after an initial drop following lyophilization and storage for up to 12 months at a temperature of 5° C. for VSV-GP-Cargo1 (FIG. 17) and VSV-GP-Cargo2 (FIG. 19) or at a temperature of 25° C. for VSV-GP-Cargo1 (FIG. 18) and VSV-GP-Cargo2 (FIG. 20) for up to 12 months. Please note that VSV-GP-Cargo1 was tested in four formulations (WP09_01-WP09_04) and VSV-GP-Cargo2 (WP9_01-WP09_06) was tested in six formulations because of limited amounts of VSV-GP-Cargo1 material. Adding more rHA (10 g/L instead of 5 g/L) or more glutamic acid (30 mM instead of 20 mM) had no further benefit. Formulations without mannitol melted upon storage of formulations containing VSV-GP-Cargo1 at a temperature of 25° C. and exhibited the greatest decrease in the infectious titer (FIG. 18) showing the cake-stabilizing effect of mannitol. The beneficial effects of poloxamer 188 in the formulation were also clearly confirmed since VSV-GP-Cargo2 formulations without poloxamer 188 had the greatest loss of infectivity when stored at a temperature of 5° C. (FIG. 19) or at 25° C. (FIG. 20) and continued to decrease over time below the limit of detection of the TCID₅₀ assay. Also, a poloxamer 188 concentration of 2.5 g/L in VSV-GP-Cargo2 formulations was sufficient to maintain the infectious titer after lyophilization (lower concentrations were not tested). These experiments revealed that the infectious titer of VSV-GP variants could be maintained after lyophilization for up to 12 months upon storage at a temperature of 25° C. following an initial drop of approximately one log unit. As expected, no differences regarding stabilizing effects were observed for VSV-GP and VSV-GP expressing a further cargo. Among the best performing compositions was 10 mM Tris (titrated with Na₃PO₄), 200 mM trehalose, 50 mM mannitol, 50 mM sorbitol, 20 mM glutamic acid, 10 g/L rHA, and 2.5 g/L poloxamer 188, pH 7.4.

### Example 4

### Fourth Optimization and Confirmation Round

The influence of poloxamer 188 and alternatively dextran 70 as a second polymer on VSV-GP stability was determined and several formulations were also subjected to a long-term stability study. In addition, the stability of VSV-GP formulations containing rHA at a reduced concentration of 5 g/L and in two formulations that were titrated with NaOH instead of phosphoric acid were examined.

TABLE 11
WP10 formulations

| | Excipient | WP10_01 | WP10_02 | WP10_03 | WP10_04 |
|---|---|---|---|---|---|
| Buffer | TRIS [mM] | 10 | 10 | 10 | 10 |
| | pH 7.4 adjustment | Na₃PO₄ | Na₃PO₄ | Na₃PO₄ | Na₃PO₄ |
| Sugars and | Trehalose [mM] | 200 | 200 | 200 | 200 |
| Poly- | | | | | |
| saccharides | Dextran | | | | 10 |
| Sugar alcohol | Mannitol [mM] | 50 | 50 | 50 | 50 |
| | Sorbitol [mM] | 50 | 50 | 50 | 50 |
| Amino acid | Glutamic acid [mM] | 20 | 20 | 20 | 30 |
| Detergent | Poloxamer 188 [g/l] | | 2.5 | 5 | 5 |
| Macromolecule | rHA [g/l] | 10 | 10 | 10 | 10 |

TABLE 11
WP10 formulations

| | Excipient | WP10_05 | WP10_06 | WP10_07 | WP10_08 | WP10_9 |
|---|---|---|---|---|---|---|
| Buffer | TRIS [mM] | 10 | 10 | 10 | 10 | 10 |
| | pH 7.4 adjustment | Na₃PO₄ | Na₃PO₄ | Na₃PO₄ | NaOH | NaOH |
| Sugars and | Trehalose [mM] | 200 | 200 | 200 | 200 | 200 |
| Polysaccharides | Dextran | | 10 | | | |
| Sugar alcohol | Mannitol [mM] | | 50 | 50 | 50 | 50 |
| | Sorbitol [mM] | 100 | 50 | 50 | 50 | 50 |
| Amino acid | Glutamic acid [mM] | 20 | 20 | 20 | 20 | 20 |
| Detergent | Poloxamer 188 [g/l] | 5 | | 5 | 5 | |
| Macromolecule | rHA [g/l] | 10 | 10 | 5 | 5 | 10 |

### Effects of Storing Formulations at a Temperature of 5° C. or 25° C. on the Infectious Titer of Lyophilized Formulations (WP10)

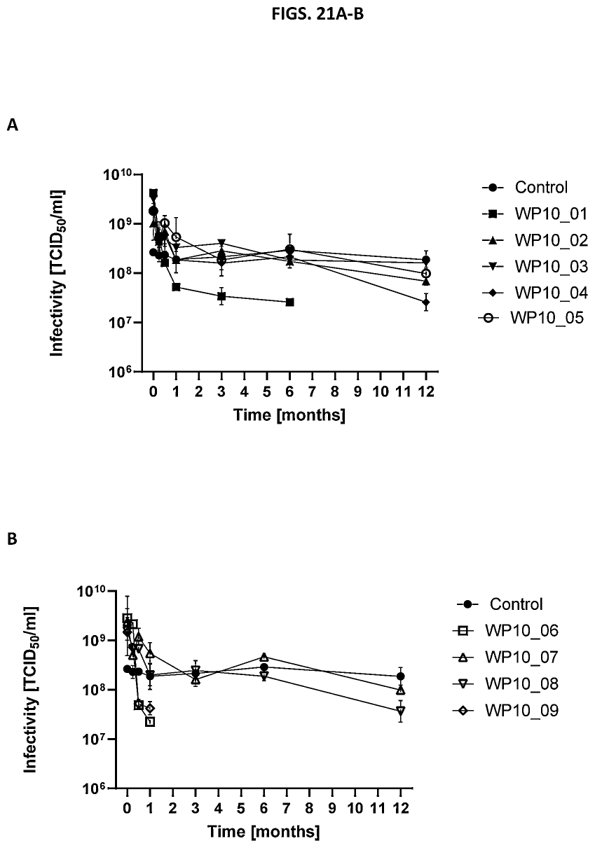

### FIGS. 21A-B

A poloxamer 188 concentration at 5 g/L in VSV-GP formulations were slightly more advantageous to maintain the infectious titer after lyophilization than a concentration at 2.5 g/L when stored after lyophilization for up to 12 months at a temperature of 25° C. for (FIGS. 21A-B). The infectious titer after lyophilization could be maintained following an initial drop of approximately one log unit for up to 12 months at a temperature of 25° C. All formulations without poloxamer 188 could not maintain the infectious titer after lyophilization following an initial drop and infectious titers continued to decrease with storage time. The presence of dextran or titration with NaOH had no benefit in stabilizing VSV-GP. The best performing composition was 10 mM Tris (phosphate), 200 mM trehalose, 50 mM mannitol, 50 mM sorbitol, 20 mM glutamic acid, 10 g/L rHA, and 5 g/L poloxamer 188, pH 7.4.

### Example 5

### Fifth Optimization Round

Additional formulation development efforts were conducted in a DoE approach to further optimize the current lead formulation and identify the optimal combinations and concentrations of stabilizing excipients for lyophilized oncolytic viruses using VSV-GP-Cargo1 based on the three months data from WP10.

TABLE 12
WP11 formulations

| | Excipient | WP11_01 | WP11_02 | WP11_03 | WP11_04 | WP11_05 |
|---|---|---|---|---|---|---|
| Buffer | TRIS [mM] | 10 | 10 | 10 | 10 | 10 |
| | pH 7.4 adjustment | Na₃PO₄ | Na₃PO₄ | Na₃PO₄ | Na₃PO₄ | Na₃PO₄ |
| Sugar | Trehalose [mM] | 200 | 200 | 200 | 200 | 200 |
| Sugar alcohol | Mannitol [mM] | 50 | 10 | 30 | 30 | 50 |
| | Sorbitol [mM] | 50 | 50 | 25 | | 50 |
| Amino acid | Glu [mM] | 20 | 20 | 20 | 20 | 20 |
| Detergent | Poloxamer 188 [g/l] | 2.5 | 0.05 | 1.275 | 0.05 | 1.275 |
| Macromolecule | rHA [g/l] | | | 2.5 | 5 | 5 |

TABLE 12
WP11 formulations

| | Excipient | WP11_06 | WP11_07 | WP11_08 | WP11_09 | WP11_10 |
|---|---|---|---|---|---|---|
| Buffer | TRIS [mM] | 10 | 10 | 10 | 10 | 10 |
| | pH 7.4 adjustment | Na₃PO₄ | Na₃PO₄ | Na₃PO₄ | Na₃PO₄ | Na₃PO₄ |
| Sugar | Trehalose [mM] | 200 | 200 | 200 | 200 | 200 |
| Sugar alcohol | Mannitol [mM] | 10 | 50 | 50 | 50 | 30 |
| | Sorbitol [mM] | | 50 | | | 50 |
| Amino acid | Glu [mM] | 20 | 20 | 20 | 20 | 20 |
| Detergent | Poloxamer 188 [g/l] | 0.05 | 0.05 | 2.5 | 0.05 | 2.5 |
| Macromolecule | rHA [g/l] | 1 | 1 | 1 | | 5 |

TABLE 12
WP11 formulations

| | Excipient | WP11_11 | WP11_12 | WP11_13 | WP11_14 | WP11_15 |
|---|---|---|---|---|---|---|
| Buffer | TRIS [mM] | 10 | 10 | 10 | 10 | 10 |
| | pH 7.4 adjustment | Na₃PO₄ | Na₃PO₄ | Na₃PO₄ | Na₃PO₄ | Na₃PO₄ |
| Sugar | Trehalose [mM] | 200 | 200 | 200 | 200 | 200 |
| Sugar alcohol | Mannitol [mM] | 50 | 50 | 50 | 10 | 30 |
| | Sorbitol [mM] | 25 | | 25 | 50 | 25 |
| Amino acid | Glu [mM] | 20 | 20 | 20 | 20 | 20 |
| Detergent | Poloxamer 188 [g/l] | 2.5 | 1.275 | 0.05 | 0.05 | 1.275 |
| Macromolecule | rHA [g/l] | 5 | 5 | 5 | 5 | 2.5 |

TABLE 12
WP11 formulations

| | Excipient | WP11_16 | WP11_17 | WP11_18 | WP11_19 | WP11_20 |
|---|---|---|---|---|---|---|
| Buffer | TRIS [mM] | 10 | 10 | 10 | 10 | 10 |
| | pH 7.4 adjustment | Na₃PO₄ | Na₃PO₄ | Na₃PO₄ | Na₃PO₄ | Na₃PO₄ |
| Sugar | Trehalose [mM] | 200 | 200 | 200 | 200 | 200 |
| Sugar alcohol | Mannitol [mM] | 30 | 10 | 10 | 10 | 10 |
| | Sorbitol [mM] | 25 | | 25 | | 50 |
| Amino acid | Glu [mM] | 20 | 20 | 20 | 20 | 20 |
| Detergent | Poloxamer 188 [g/l] | 1.275 | 2.5 | 1.275 | 2.5 | 2.5 |
| Macromolecule | rHA [g/l] | | | 5 | 5 | 1 |

### Effects of Storing Formulations at a Temperature of 25° C. on the Infectious Titer of Lyophilized Formulations in a DoE Approach (WP11)

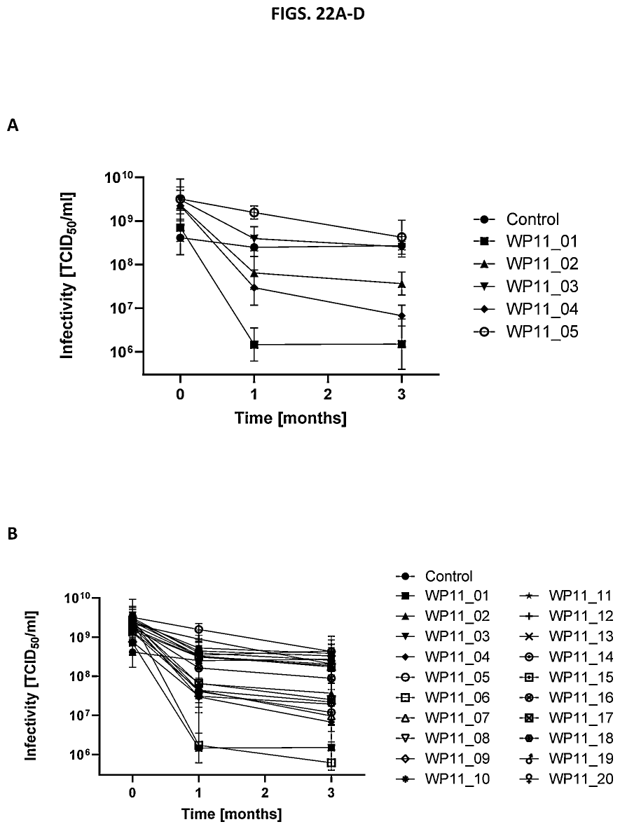

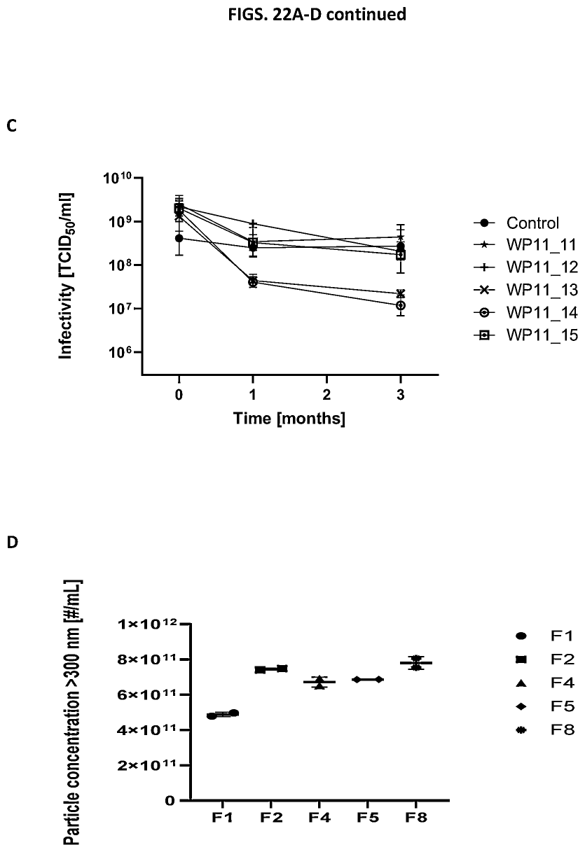

### FIGS. 22A-D

In this round, a design of experiment (DoE) approach was used to evaluate if the formulation could be further optimized. The variable concentrations were those of mannitol, sorbitol, poloxamer 188, and rHA. Concentrations of Tris, trehalose, and glutamic acid were fixed. In essence, results of the traditional one-factor-at-a-time formulation development approach were confirmed by the DoE approach (FIGS. 22A-D. Using different optimization methods with the response surface model, the DoE predicted the following optimum formulations within the WP11 design space: 10 mM Tris (phosphate), 200 mM trehalose, 20-46 mM mannitol, 20-50 mM sorbitol, 20 mM glutamic acid, 3-5 g/L rHA, and 1.4-1.5 g/L poloxamer 188, pH 7.4.

TABLE 13
WP11 DoE predicted formulations within the WP11 design space following response surface modelling

| | | WP11_DoE | WP11_DoE | WP11_DoE | WP11_DoE | WP11_DoE |
|---|---|---|---|---|---|---|
| | Excipient | 1a | 2a | 3a | 4a | 5a |
| Buffer | TRIS [mM] | 10 | 10 | 10 | 10 | 10 |
| | pH 7.4 adjustment | Na₃PO₄ | Na₃PO₄ | Na₃PO₄ | Na₃PO₄ | Na₃PO₄ |
| Sugar | Trehalose [mM] | 200 | 200 | 200 | 200 | 200 |
| Sugar alcohol | Mannitol [mM] | 34.6 | 20.7 | 45.5 | 20.1 | 20.2 |
| | Sorbitol [mM] | 49.6 | 49 | 20.3 | 21.5 | 21.6 |
| Amino acid | Glu [mM] | 20 | 20 | 20 | 20 | 20 |
| Detergent | Poloxamer 188 [g/l] | 1.4 | 1.4 | 1.4 | 1.4 | 1.4 |
| Macromolecule | rHA [g/l] | 3.2 | 4.5 | 5.0 | 4.3 | 4.3 |

The DoE predicted the following optimum formulations outside the WP11 design space based on different optimization methods: 10 mM Tris (phosphate), 200 mM trehalose, 20-69 mM mannitol, 19-76 mM sorbitol, 20 mM glutamic acid, 3.4-5.6 g/L rHA, and 1.4-1.5 g/L poloxamer 188, pH 7.4.

TABLE 14
WP11 DoE predicted formulations outside the WP11 design space following response surface modelling

| | | WP11_DoE | WP11_DoE | WP11_DoE | WP11_DoE | WP11_DoE |
|---|---|---|---|---|---|---|
| | Excipient | 1b | 2b | 3b | 4b | 5b |
| Buffer | TRIS [mM] | 10 | 10 | 10 | 10 | 10 |
| | pH 7.4 adjustment | Na₃PO₄ | Na₃PO₄ | Na₃PO₄ | Na₃PO₄ | Na₃PO₄ |
| Sugar | Trehalose [mM] | 200 | 200 | 200 | 200 | 200 |
| Sugar alcohol | Mannitol [mM] | 35.4 | 21.2 | 68.9 | 21.1 | 21.2 |
| | Sorbitol [mM] | 72.7 | 76.4 | 19.1 | 21.5 | 21.6 |
| Amino acid | Glu [mM] | 20 | 20 | 20 | 20 | 20 |
| Detergent | Poloxamer 188 [g/l] | 1.4 | 1.4 | 1.5 | 1.4 | 1.4 |
| Macromolecule | rHA [g/l] | 3.4 | 4.7 | 5.6 | 4.3 | 4.3 |

TABLE 15
Selected formulations of previous work packages

| | | | | WP11_05 |
|---|---|---|---|---|
| | | | | (previously |
| | Excipient | WP10_03/05/07 | WP09a_01/02 | best) |
| Buffer | TRIS [mM] | 10 | 10 | 10 |
| | pH 7.4 adjustment | Na₃PO₄ | Na₃PO₄ | Na₃PO₄ |
| Sugar | Trehalose [mM] | 200 | 200 | 200 |
| Sugar alcohol | Mannitol [mM] | 0-50 | 50 | 50 |
| | Sorbitol [mM] | 50-100 | 50 | 50 |
| Amino acid | Glu [mM] | 20 | 20 | 20 |
| Detergent | Poloxamer 188 | 5 | 5 | 1.275 |
| | [g/l] | | | |
| Macromolecule | rHA [g/l] | 5-10 | 5-10 | 5 |

### Example 6

### Enhancing Colloidal Stability

Based on outcome of the DoE, additional studies were performed adding different excipients potentially acting as colloidal stabilizers to further optimized formulations and being able to increase overall particle counts and stability during liquid processing.

TABLE 16
Formulations based on best-performing formulation from DoE
(WP11_05) containing various Citrate or L-Arginine concentrations.

| | Excipient | 26_F1 | 26_F2 | 26_F4 | 26_F5 | 26_F8 |
|---|---|---|---|---|---|---|
| Buffer | TRIS [mM] | 10 | 10 | 10 | 10 | 10 |
| | Citrate [mM] | | 75 | 25 | 50 | |
| | pH adjustment | 7.4 | 7.4 | 7.4 | 7.1 | 7.1 |
| | H3PO4/Na3PO4 | | | | | |
| Sugar | Trehalose [mM] | 200 | 200 | 200 | 125 | 125 |
| Sugar alcohol | Mannitol [mM] | 50 | 50 | 50 | 100 | 100 |
| | Sorbitol [mM] | 50 | 50 | 50 | 40 | 40 |
| Amino acid | Glu [mM] | 20 | 20 | 20 | 20 | 20 |
| | Arg [mM] | | | | | 100 |
| Detergent | Poloxamer 188 [g/l] | 1.275 | 1.275 | 1.275 | 2.5 | 2.5 |
| Macromolecule | rHA [g/l] | 5 | 5 | 5 | 5 | 5 |

### Additional Excipients to Enhance Recovery of Virus During Liquid Handling and Improve Liquid Processing w/o Negatively Impacting Long-Term Storage Stability in Dried Conditions

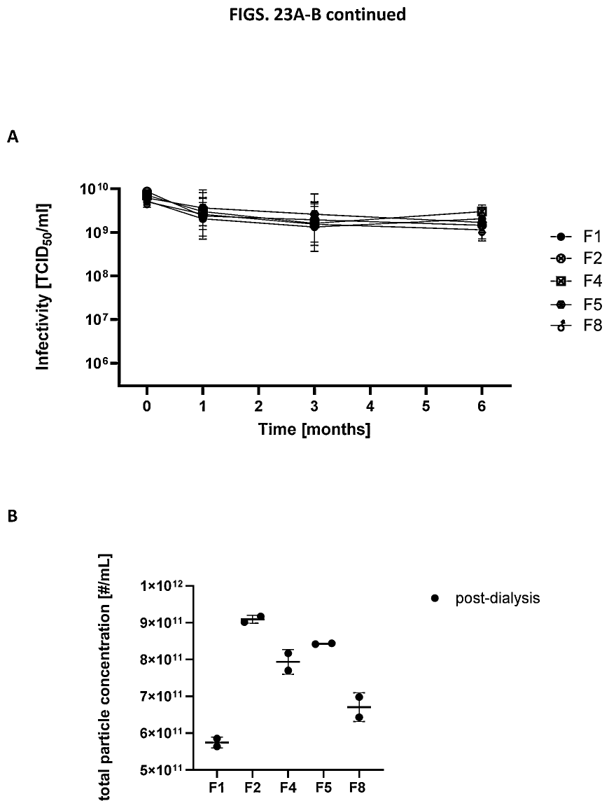

### FIGS. 23A-B

Starting from same VSV-GP starting material in presence of additional excipients (e.g. Arginine or Citrate) known to act as colloidal stabilizer higher overall particle counts were achieved during liquid handling and processing. Whereas stability is confirmed after dry storage for up to 6 months at a temperature of 2-8° C.

1. A pharmaceutical composition comprising an enveloped virus, one or more sugar alcohol, and a protein agent and/or a poly(ethylene oxide)/poly(propylene oxide) block copolymer.

2. The pharmaceutical composition of claim 1, wherein the poly(ethylene oxide) and poly(propylene oxide) block copolymer is a poloxamer.

3. The pharmaceutical composition of claim 1, wherein the poly(ethylene oxide) and poly(propylene oxide) block copolymer is poloxamer 188.

4. The pharmaceutical composition of claim 1, wherein the protein agent is albumin or gelatin.

5. The pharmaceutical composition of claim 1, wherein the protein agent is human serum albumin or recombinant human albumin.

6. The pharmaceutical composition of claim 1, wherein the poly(ethylene oxide) and poly(propylene oxide) block copolymer is poloxamer 188, and the protein agent is human serum albumin or recombinant human albumin.

7. The pharmaceutical composition of claim 6, comprising poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

8. The pharmaceutical composition of claim 6, comprising human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

9. The pharmaceutical composition according to claim 1, further comprising at least one of an amino acid, a buffer, or a sugar.

10. The pharmaceutical composition of claim 1, wherein the amino acid is selected from the group consisting of alanine, arginine, phenylalanine, glutamic acid, glycine, methionine, lysine, and glutamine, preferably glutamic acid and/or arginine.

11. The pharmaceutical composition of claim 9, wherein the buffer is selected from the group consisting of acetate, citrate, histidine, succinate, HEPES, tartrate, phosphate, citrate/phosphate, and Tris, preferably Tris.

12. The pharmaceutical composition of claim 9, wherein the sugar is sucrose or trehalose, preferably trehalose.

13. The pharmaceutical composition of claim 1, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate.

14. The pharmaceutical composition of claim 13, wherein the one or more sugar alcohol is mannitol and/or sorbitol, preferably a combination of mannitol and sorbitol.

15. The pharmaceutical composition of claim 9, wherein the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM.

16. The pharmaceutical composition of claim 9, wherein the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

17. The pharmaceutical composition of claim 1, wherein the composition is substantially free of chloride, preferably substantially free of NaCl.

18. The pharmaceutical composition of claim 1, wherein the pH of the composition is between 5 to 9, or between 6 to 9, or between 6.5 to 8.5, or between 6.5 to 8.0, preferably between 7.0 and 8.0.

19. The pharmaceutical composition of claim 18, wherein the pH of the composition is adjusted with phosphoric acid, lactic acid, citric acid, succinate acid or their respective salts.

20. The pharmaceutical composition according to claim 1, comprising:
i) an enveloped virus,
ii) one or more sugar alcohol,
iii) at least one of a buffer, an amino acid or a sugar, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

21. The pharmaceutical composition according to claim 1, comprising:
i) an enveloped virus,
ii) one or more sugar alcohol,
iii) an amino acid and a buffer, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

22. The pharmaceutical composition according to claim 1, comprising:
i) an enveloped virus,
ii) one or more sugar alcohol,
iii) an amino acid and a sugar, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

23. The pharmaceutical composition according to claim 1, comprising:
i) an enveloped virus,
ii) one or more sugar alcohol,
iii) an amino acid and a buffer and a sugar, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

24. The pharmaceutical composition according to claim 1, comprising:
i) an enveloped virus,
ii) one or more sugar alcohol,
iii) a buffer and a sugar, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

25. The pharmaceutical composition according to claim 1 comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) at least one of a buffer, an amino acid or a sugar, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

26. The pharmaceutical composition according to claim 1, comprising:
i) an enveloped virus,
ii) one or more sugar alcohol,
iii) at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

27. The pharmaceutical composition according to claim 1, comprising:
i) an enveloped virus,
ii) one or more sugar alcohol,
iii) an amino acid and a buffer, wherein the buffer is selected from the group consisting of acetate, citrate, histidine, succinate, HEPES, tartrate, phosphate, citrate/phosphate, lactate and Tris, preferably Tris, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

28. The pharmaceutical composition according to claim 1, comprising:
i) an enveloped virus,
ii) one or more sugar alcohol,
iii) an amino acid and a buffer and a sugar, wherein the buffer is selected from the group consisting of acetate, citrate, histidine, succinate, HEPES, tartrate, phosphate, citrate/phosphate, lactate, and Tris, preferably Tris, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

29. The pharmaceutical composition according to claim 1, comprising:
i) an enveloped virus,
ii) one or more sugar alcohol,
iii) a buffer and a sugar, wherein the buffer is selected from the group consisting of acetate, citrate, histidine, succinate, HEPES, tartrate, phosphate, citrate/phosphate, lactate, and Tris, preferably Tris, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

30. The pharmaceutical composition according to claim 1, comprising:
i) an enveloped virus,
ii) one or more sugar alcohol,
iii) at least one of a buffer, an amino acid or a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

31. The pharmaceutical composition according to claim 1, comprising:
i) an enveloped virus,
ii) one or more sugar alcohol,
iii) an amino acid and a buffer, wherein the amino acid is selected from the group consisting of alanine, arginine, phenylalanine, glutamic acid, glycine, methionine, lysine, and glutamine, preferably glutamic acid and/or arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

32. The pharmaceutical composition according to claim 1, comprising:
i) an enveloped virus,
ii) one or more sugar alcohol,
iii) an amino acid and a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

33. The pharmaceutical composition according to claim 1, comprising:
i) an enveloped virus,
ii) one or more sugar alcohol,
iii) an amino acid and a buffer and a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

34. The pharmaceutical composition according to claim 1, comprising:
i) an enveloped virus,
ii) one or more sugar alcohol,
iii) a buffer and a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

35. The pharmaceutical composition according to claim 1, comprising:
i) an enveloped virus,
ii) one or more sugar alcohol,
iii) at least one of a buffer, an amino acid or a sugar, wherein the amino acid is selected from the group consisting of alanine, arginine, phenylalanine, glutamic acid, glycine, methionine, lysine, and glutamine, preferably glutamic acid and/or arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

36. The pharmaceutical composition according to claim 1, comprising:
i) an enveloped virus,
ii) one or more sugar alcohol,
iii) an amino acid and a sugar, wherein the amino acid is selected from the group consisting of alanine, arginine, phenylalanine, glutamic acid, glycine, methionine, lysine, and glutamine, preferably glutamic acid and/or arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

37. The pharmaceutical composition according to claim 1, comprising:
i) an enveloped virus,
ii) one or more sugar alcohol,
iii) an amino acid and a buffer and a sugar, wherein the amino acid is selected from the group consisting of alanine, arginine, phenylalanine, glutamic acid, glycine, methionine, lysine, and glutamine, preferably glutamic acid and/or arginine, and iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

38. The pharmaceutical composition according to claim 1, comprising:
i) an enveloped virus,
ii) one or more sugar alcohol,
iii) at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

39. The pharmaceutical composition according to claim 1, comprising:
i) an enveloped virus,
ii) one or more sugar alcohol,
iii) an amino acid and a buffer, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

40. The pharmaceutical composition according to claim 1, comprising:
i) an enveloped virus,
ii) one or more sugar alcohol,
iii) an amino acid and a sugar, wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

41. The pharmaceutical composition according to claim 1, comprising:
i) an enveloped virus,
ii) one or more sugar alcohol,
iii) an amino acid and a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

42. The pharmaceutical composition according to claim 1, comprising:
i) an enveloped virus,
ii) one or more sugar alcohol,
iii) a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

43. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) at least one of a buffer, an amino acid or a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

44. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a buffer, wherein the amino acid is selected from the group consisting of alanine, arginine, phenylalanine, glutamic acid, glycine, methionine, lysine, and glutamine, preferably glutamic acid and/or arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

45. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

46. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a buffer and a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

47. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) a buffer and a sugar, wherein the sugar is selected from the group consisting of dextrose, fructose, galactose, glucose, raffinose, trehalose, and sucrose, preferably trehalose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

48. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) at least one of a buffer, an amino acid or a sugar, wherein the amino acid is selected from the group consisting of alanine, arginine, phenylalanine, glutamic acid, glycine, methionine, lysine, and glutamine, preferably glutamic acid and/or arginine, and
wherein the protein agent is selected from human serum albumin or recombinant human albumin and the poly(ethylene oxide)/poly(propylene oxide) block copolymer is poloxamer 188.

49. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a sugar, wherein the amino acid is selected from the group consisting of alanine, arginine, phenylalanine, glutamic acid, glycine, methionine, lysine, and glutamine, preferably glutamic acid and/or arginine, and
wherein the protein agent is selected from human serum albumin or recombinant human albumin and the poly(ethylene oxide)/poly(propylene oxide) block copolymer is poloxamer 188.

50. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a buffer and a sugar, wherein the amino acid is selected from the group consisting of alanine, arginine, phenylalanine, glutamic acid, glycine, methionine, lysine, and glutamine, preferably glutamic acid and/or arginine, and
wherein the protein agent is selected from human serum albumin or recombinant human albumin and the poly(ethylene oxide)/poly(propylene oxide) block copolymer is poloxamer 188.

51. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose, and
wherein the protein agent is selected from human serum albumin or recombinant human albumin and the poly(ethylene oxide)/poly(propylene oxide) block copolymer is poloxamer 188.

52. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a buffer, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine, and
wherein the protein agent is selected from human serum albumin or recombinant human albumin and the poly(ethylene oxide)/poly(propylene oxide) block copolymer is poloxamer 188.

53. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a sugar, wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose, and
wherein the protein agent is selected from human serum albumin or recombinant human albumin and the poly(ethylene oxide)/poly(propylene oxide) block copolymer is poloxamer 188.

54. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) an amino acid and a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine; wherein the sugar is selected from the group consisting of dextrose, fructose, galactose, glucose, raffinose, trehalose, and sucrose, preferably trehalose, and
wherein the protein agent is selected from human serum albumin or recombinant human albumin and the poly(ethylene oxide)/poly(propylene oxide) block copolymer is poloxamer 188.

55. The pharmaceutical composition according to claim 1, comprising:
i) an enveloped virus,
ii) one or more sugar alcohol, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate, preferably mannitol and/or sorbitol, more preferably a combination of mannitol and sorbitol,
iii) a buffer and a sugar, wherein the buffer is selected from the group consisting of acetate, citrate, histidine, succinate, HEPES, tartrate, phosphate,
citrate/phosphate, lactate and Tris, preferably Tris; wherein the amino acid is selected from the group consisting of alanine, arginine, phenylalanine, glutamic acid, glycine, methionine, lysine, and glutamine, preferably glutamic acid and/or arginine, and
wherein the protein agent is selected from human serum albumin or recombinant human albumin and the poly(ethylene oxide)/poly(propylene oxide) block copolymer is poloxamer 188.

56. The pharmaceutical composition according to claim 1, comprising:
i) an enveloped virus,
ii) mannitol and/or sorbitol
iii) a buffer, wherein the buffer is Tris, and
wherein the protein agent is selected from human serum albumin or recombinant human albumin and the poly(ethylene oxide)/poly(propylene oxide) block copolymer is poloxamer 188.

57. The pharmaceutical composition according to claim 1, comprising:
i) an enveloped virus,
ii) mannitol and/or sorbitol,
iii) an amino acid, wherein the amino acid is arginine or glutamic acid, and
wherein the protein agent is selected from human serum albumin or recombinant human albumin and the poly(ethylene oxide)/poly(propylene oxide) block copolymer is poloxamer 188.

58. The pharmaceutical composition according to claim 1, comprising:
i) an enveloped virus,
ii) mannitol and/or sorbitol
iii) a sugar, wherein the sugar is trehalose or sucrose, and
wherein the protein agent is selected from human serum albumin or recombinant human albumin and the poly(ethylene oxide)/poly(propylene oxide) block copolymer is poloxamer 188.

59. The pharmaceutical composition according to claim 1, comprising:
i) an enveloped virus,
ii) mannitol and/or sorbitol
iii) an amino acid, wherein the amino acid is arginine or glutamic acid,
iv) a sugar, wherein the sugar is trehalose or sucrose, and
wherein the protein agent is selected from human serum albumin or recombinant human albumin and the poly(ethylene oxide)/poly(propylene oxide) block copolymer is poloxamer 188.

60. The pharmaceutical composition according to claim 1, comprising:
i) an enveloped virus,
ii) mannitol and/or sorbitol
iii) a buffer, wherein the buffer is Tris
iv) a sugar, wherein the sugar is trehalose or sucrose, and
wherein the protein agent is selected from human serum albumin or recombinant human albumin and the poly(ethylene oxide)/poly(propylene oxide) block copolymer is poloxamer 188.

61. The pharmaceutical composition according to claim 1, comprising:
i) an enveloped virus,
ii) mannitol and/or sorbitol
iii) a buffer, wherein the buffer is Tris,
iv) an amino acid, wherein the amino acid is arginine or glutamic acid, and
wherein the protein agent is selected from human serum albumin or recombinant human albumin and the poly(ethylene oxide)/poly(propylene oxide) block copolymer is poloxamer 188.

62. The pharmaceutical composition according to claim 1, comprising:
i) an enveloped virus,
ii) mannitol and/or sorbitol
iii) a buffer, wherein the buffer is Tris,
iv) an amino acid, wherein the amino acid is arginine or glutamic acid,
v) a sugar, wherein the sugar is trehalose or sucrose, and
wherein the protein agent is selected from human serum albumin or recombinant human albumin and the poly(ethylene oxide)/poly(propylene oxide) block copolymer is poloxamer 188.

63. The pharmaceutical composition of claim 21, wherein the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM.

64. The pharmaceutical composition of claim 20, wherein the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

65. The pharmaceutical composition of claim 20, comprising poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

66. The pharmaceutical composition of claim 20, comprising human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

67. The pharmaceutical composition of claim 1, comprising:
i) an enveloped virus,
ii) about 1-100 mM Tris,
iii) about 1-100 mM glutamic acid,
iv) about 10-500 mM trehalose,
v) about 10-200 mM mannitol,
vi) about 10-200 mM sorbitol,
vii) about 0.1-5 mg/ml poloxamer 188,
viii) about 0.5-10 mg/ml recombinant human albumin, and
ix) a pH of about 6 to 8.

68. The pharmaceutical composition of claim 1, comprising:
i) an enveloped virus,
ii) about 1-100 mM Tris,
iii) about 1-100 mM glutamic acid,
iv) about 10-500 mM arginine,
v) about 10-500 mM trehalose,
vi) about 10-200 mM mannitol,
vii) about 10-200 mM sorbitol,
viii) about 0.1-5 mg/ml poloxamer 188,
ix) about 0.5-10 mg/ml recombinant human albumin, and
x) a pH of about 6 to 8.

69. The pharmaceutical composition of claim 1, comprising:
i) an enveloped virus,
ii) about 1-100 mM Tris,
iii) about 1-100 mM glutamic acid,
iv) about 10-500 mM trehalose,
v) about 1-100 mM citrate,
vi) about 10-200 mM mannitol,
vii) about 10-200 mM sorbitol,
viii) about 0.1-5 mg/ml poloxamer 188,
ix) about 0.5-10 mg/ml recombinant human albumin, and
x) a pH of about 6 to 8.

70. The pharmaceutical composition of claim 1, comprising:
i) an enveloped virus,
ii) about 1-100 mM Tris,
iii) about 1-100 mM glutamic acid,
iv) about 10-500 mM trehalose,
v) about 10-200 mM mannitol,
vi) about 10-200 mM sorbitol,
vii) about 0.1-5 mg/ml poloxamer 188,
viii) about 0.5-10 mg/ml recombinant human albumin, and
ix) a pH of about 6 to 8.

71. The pharmaceutical composition of claim 67, comprising:
i) an enveloped virus
ii) about 10 mM Tris,
iii) about 200 mM trehalose,
iv) about 20 mM glutamic acid,
v) about 50 mM mannitol,
vi) about 50 mM sorbitol,
vii) about 1.275 mg/ml poloxamer 188,
viii) about 5 mg/ml recombinant human albumin, and
ix) a pH of about 7.4.

72. The pharmaceutical composition of claim 68, comprising:
i) an enveloped virus
ii) about 10 mM Tris,
iii) about 125 mM trehalose,
iv) about 20 mM glutamic acid,
v) about 100 mM mannitol,
vi) about 40 mM sorbitol,
vii) about 100 mM arginine,
viii) about 2.5 mg/ml poloxamer 188,
ix) about 5 mg/ml recombinant human albumin, and
x) a pH of about 7.1.

73. The pharmaceutical composition of claim 69, comprising:
i) an enveloped virus,
ii) about 10 mM Tris,
iii) about 20 mM glutamic acid,
iv) about 200 mM trehalose,
v) about 25 mM citrate,
vi) about 50 mM mannitol,
vii) about 50 mM sorbitol,
viii) about 2.5 mg/ml poloxamer 188,
ix) about 5 mg/ml recombinant human albumin, and
x) a pH of about 7.4.

74. The pharmaceutical composition of claim 68, comprising:
i) an enveloped virus
ii) about 10 mM Tris,
iii) about 200 mM trehalose,
iv) about 20 mM glutamic acid,
v) about 50 mM mannitol,
vi) about 50 mM sorbitol,
vii) about 150 mM arginine,
viii) about 1.275 mg/ml poloxamer 188,
ix) about 5 mg/ml recombinant human albumin, and
x) a pH of about 7.4.

75. The pharmaceutical composition of claim 68, comprising:
i) an enveloped virus
ii) about 10 mM Tris,
iii) about 133 mM trehalose,
iv) about 13 mM glutamic acid,
v) about 33 mM mannitol,
vi) about 33 mM sorbitol,
vii) about 100 mM arginine,
viii) about 1.275 mg/ml poloxamer 188,
ix) about 5 mg/ml recombinant human albumin, and
x) a pH of about 7.4.

76. The pharmaceutical composition of claim 67, wherein the pH of the composition is adjusted with phosphoric acid or sodium phosphate or lactic acid.

77. The pharmaceutical composition according to claim 1, wherein the enveloped virus is a rhabdoviridae, preferably a vesiculovirus or vesicular stomatitis virus (VSV).

78. The pharmaceutical composition according to claim 77, wherein the enveloped virus is a recombinant vesicular stomatitis virus (VSV), wherein the gene coding for the glycoprotein G of the vesicular stomatitis virus is replaced by the gene coding for the glycoprotein GP of LCMV, and/or the glycoprotein G is replaced by the glycoprotein GP of LCMV.

79. The pharmaceutical composition according to claim 77, wherein the pharmaceutical composition comprises the enveloped virus, preferably the vesiculovirus or vesicular stomatitis virus (VSV), in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, at least 1×10⁹ TCID₅₀/mL, or at least 1×10¹⁰ TCID₅₀/mL.

80. The pharmaceutical composition according to claim 77, wherein the pharmaceutical composition comprises the enveloped virus, preferably the vesiculovirus or vesicular stomatitis virus (VSV), in a concentration range between 1×10⁵ TCID₅₀/mL to 1×10¹² TCID₅₀/mL, between 1×10⁶ TCID₅₀/mL to 1×10¹² TCID₅₀/mL, between 1×10⁷ TCID₅₀/mL to 1×10¹² TCID₅₀/mL, between 1×10⁸ TCID₅₀/mL to 1×10¹¹ TCID₅₀/mL, between 1×10⁸ TCID₅₀/mL to 1×10¹² TCID₅₀/mL, 1×10⁵ TCID₅₀/mL to 1×10¹¹ TCID₅₀/mL, 1×10⁵ TCID₅₀/mL to 1×10¹⁰ TCID₅₀/mL, or 1×10⁵ TCID₅₀/mL to 1×10⁹ TCID₅₀/mL.

81. A pharmaceutical composition according to claim 1, wherein the pharmaceutical composition is a liquid or frozen liquid pharmaceutical composition.

82. A dry pharmaceutical composition produced by a method comprising removing water from a pharmaceutical composition according to claim 1.

83. A pharmaceutical composition according to claim 1, wherein the pharmaceutical composition is a dry pharmaceutical composition.

84. The dry pharmaceutical composition according to claim 82, wherein the dry pharmaceutical composition is obtained by freeze-drying/lyophilization, or spray-drying.

85. The dry pharmaceutical composition of claim 84, wherein the method comprises placing the pharmaceutical composition in a vacuum under controlled temperatures and pressure to remove the water.

86. The dry pharmaceutical composition according to claim 84, wherein the method is lyophilization.

87. The dry pharmaceutical composition according to claim 84, comprising less than about (0.1%-10%) w/w water.

88. A pharmaceutical composition comprising water and the dry pharmaceutical composition of claim 81.

89. A lyophilized formulation that, when reconstituted in water, results in a reconstituted solution comprising:
i) an enveloped virus,
ii) mannitol in a concentration between 10 mM to 200 mM and/or sorbitol in a concentration between 10 mM to 200 mM, and
iii) poloxamer 188 in a concentration between 0.01 g/L to 50 g/L and/or recombinant human albumin in a concentration between 0.1 g/L to 50 g/L.


SEQUENCE LISTING (8 sequences)

SEQ ID NO: 1 - protein, 422 aa, Vesicular stomatitis virus
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 2 - protein, 265 aa, Vesicular stomatitis virus
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 3 - protein, 2109 aa, Vesicular stomatitis virus
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 4 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 5 - protein, 498 aa, Lymphocytic choriomeningitis mammarenavirus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 6 - RNA, 11169 nt, Vesicular stomatitis virus
  source 1..11169: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctcttaattaacgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca

SEQ ID NO: 7 - RNA, 11520 nt, Vesicular stomatitis virus
  source 1..11520: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtacccgaccaggacgtagtaagacaaagaccaccggtgtcggtggccacaggtatcaagactaccgccgcgagtcctgacgacggacttcatgagagtctctttctaggggcggttccagcacgcctcgatgtctttcgttctcggaagagacccgacgagatagggacggtaggacaaagacggggccttctctagtgtccggcttgacacgcggctaggatttcttgacacccacgtcgtcgactacgtcgtagacctgttctggggtagaggagtcttcggacgagttccgactactaaaaatatgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagcatcggacgagttccgact

SEQ ID NO: 8 - RNA, 12603 nt, Vesicular stomatitis virus
  source 1..12603: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtacccggtgtgttcttccgtcccttgttcggggtcgttcacggggatggacttgaagaaagtcgacgaccacgaccggccggactcggtgaaaacaagaccgcactaggtgcactggtttcttcactttctccagcggtgtgactcgacgccggtgttgcaaagtcaccttcttgaccgggtctggtcttagatgaccgtctttcttttcttttaccacgactggtactactcgccgctgtacttgtagaccgggctcatgttcttggcctggtagaagctgtagtggttgttggactcgtagcactaagaccgggactccggaagactactcccgtggatactcacgcaccacgacttcatgctcttcctgcggaagttcgcgctcgtagaccggcttcactgtgactcgcacttccggctgaaagggtgtggatcgtagtcgctgaagctctaggggtggtcgttgtagtctgcctagtagacatcgtggtcgccgccgaaaggtctcggagtagacagaaccgaccttttgccgctccttgacttgcggtagttgtggtggcacagagtcctggggctctgtctcgacatacggcacaggtcgttcgacctgaagttgtactggtggttggtgtcgaagtacacggactaattcatgccggtggactctcacttggtctggaagttgaccttgtggtggttcgttctcgtgaaggggctgctgttctgggtgtggacaggaggtacaggacgaggtcttgacgagccgcctgggaggcacaaagacaagggaggtttcggattcctgtgggactactagtcgtcttggggacttcactggacgcaccaccacctacacagagtgctcctgggtcttcacttcaagttaaccatgcacctgccgcaccttcacgtgttgcggttctggttcggatctctccttgtcatgttgtcgtggatgtctcaccacaggcacgactggcacgacgtagtcctgaccgacttgccgtttctcatgttcacgttccacaggttgttccgggacggacgaggatagctcttttggtagtcgttccggttccctgtcggggcccttggagttcacatatgggacggaggatcggcccttctctactggttcttagtccacagggactggacggagcacttcccgaagatgggaaggctatagcggcaccttaccctctcgttaccggtcggactcttgttgatgttctgttggggaggacacgacctgtcgctgccgagtaagaaggacatgtcgtttgactggcacctgttctcgtctaccgtcgtcccgttacacaagtcgacgtcgcactacgtgctccgggacgtgttggtgatgtgggtcttttcggactcggacagaggaccgactagttgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca